(19) United States
(12) Patent Application Publication
(10) Pub. No.: US 2019/0017032 A1
(43) Pub. Date: Jan. 17, 2019

(54) CELL REPROGRAMMING

(71) Applicants: Monash University, Clayton, Victoria (AU); The University of Bristol, Bristol (GB); RIKEN, Saitama (JP); Cell Mogrify Limited, Cardiff (GB)

(72) Inventors: Jaber Firas, Clayton, Victoria (AU); Jose Polo, Clayton, Victoria (AU); Julian Gough, Bristol (GB); Yoshihide Hayashizaki, Saitama (JP); Owen Rackham, Cardiff, Wales (GB)

(21) Appl. No.: 16/064,905

(22) PCT Filed: Dec. 23, 2016
(86) PCT No.: PCT/AU2016/051287

§ 371 Date: Jun. 21, 2018

(30) Foreign Application Priority Data
Dec. 23, 2015 (AU) 2015905349

(51) Int. Cl. C12N 5/074; G06F 19/12; G06F 19/22; C12N 5/0775; C12N 15/867

(52) U.S. Cl. CPC C12N 5/0696; G06F 19/12; G06F 19/22; C12N 2510/00; C12N 5/0667; C12N 15/867; C12N 2501/60; C12N 5/0663

(57) ABSTRACT

The invention relates to methods and compositions for converting one cell type to another cell type. Specifically, the invention relates to transdifferentiation of a cell to a different cell type. The invention relates to a method for determining the transcription factors required for conversion of a source cell to a cell exhibiting at least one characteristic of a target cell type. The invention also relates to method of reprogramming or forward programming a source cell.

This application claims priority from Australian provisional application AU 2015905349, the entire disclosure of which is herein incorporated in its entirety.

### FIELD OF THE INVENTION

The invention relates to methods and compositions for converting one cell type to another cell type. Specifically, the invention relates to transdifferentiation of a cell to a different cell type.

### BACKGROUND OF THE INVENTION

Cell-based regenerative therapy requires the generation of specific cell types for replacing tissues damaged by injury, disease or age. Embryonic stem cells (ESC) have the potential to differentiate in every cell type from the (human) body and have therefore been extensively studied as a source for replacement therapy. However, ESC cannot be derived in a patient-specific fashion since they are established from cultured blastocysts. Therefore, immune rejection and ethical concerns are the main barriers that prevent the transfer of the ESC technology, and in particular of human ESC technology, to clinical applications.

Cell-replacement therapies have the potential to rapidly generate a variety of therapeutically important cell types directly from one's own easily accessible tissues, such as skin or blood. Such immunologically-matched cells would also pose less risk for rejection after transplantation. Moreover, these cells would manifest less tumorigenicity since they are terminally differentiated.

Trans-differentiation, the process of converting from one cell type to another without going through a pluripotent state, may have great promise for regenerative medicine but has yet to be reliably applied. Although it may be possible to switch the phenotype of one somatic cell type to another, the elements required for conversion are difficult to identify and in most instances unknown. The identification of factors to directly reprogram the identity of cell types is currently limited by, amongst other things, the cost of exhaustive experimental testing of plausible sets of factors, an approach that is inefficient and unscalable.

There is a need for a new and/or improved method for identifying the factors required to convert one cell type to another. There is also a need for cells and cell populations for use in therapeutic applications.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention relates to a predictive framework that combines gene expression data with regulatory network information to predict the reprogramming factors necessary to induce cell conversion (i.e. convert a source cell to a cell displaying characteristic of a target cell type). This framework correctly predicts transcription factors used in known transdifferentiations as well as transcription factors for previously unknown transdifferentiations that have been experimentally validated. The present invention also relates to methods and compositions for direct reprogramming (i.e. transdifferentiation or cellular reprogramming) of a source cell to a cell having characteristics of a target cell type.

The present invention provides a method for determining the transcription factors required for conversion of a source cell to a cell exhibiting at least one characteristic of a target cell type, the method comprising the steps of:
determining differential expression of genes in the source and target cell types;
determining a network score for each transcription factor (TF) in each of the source and target cell types based on the differential gene expression over at least one network, wherein the network contains information of interactions that affect gene expression;
ranking the TFs based on a combination of network scores and differential gene expression information, thereby identifying the set of transcription factors for a conversion from a source cell to a cell exhibiting at least one characteristic of a target cell type.

The present invention provides a method for determining the transcription factors required for conversion of a source cell to a cell exhibiting at least one characteristic of a target cell type, the method comprising the steps of:
determining a gene score for each differentially expressed gene in the source and target cell types;
determining a network score for each transcription factor (TF) in each of the source and target cell types by performing a weighted sum of each gene score over at least one network, wherein the network contains information of interactions that affect gene expression;
ranking the TFs based on a combination of gene and network scores; and
identifying the set of transcription factors for a conversion from a source cell to a cell exhibiting at least one characteristic of a target cell type based on comparisons of the ranked lists for each cell type.

Preferably, the gene score is a combination of the log fold change and adjusted P− value of the differential expression. The gene score may be calculated using a tree-based method or Bayesian clustering.

Preferably, the network contains information of protein-DNA interactions, protein-DNA, protein-RNA interactions. Typically, the network contains information of the interaction between transcription factors and regulatory regions of a gene. Typically, the regulatory region is a promoter region of a gene.

Preferably, the method further comprises the step of collecting expression data for each gene prior to determining a gene score.

Preferably, the method further comprises the step of removing transcriptionally redundant TFs from the ranked lists from each cell type.

The present invention provides a method for determining the transcription factors required for conversion of a source cell to a cell exhibiting at least one characteristic of a target cell type, the method comprising the steps of:
collecting expression data for each gene in the source cell type and target cell type;
calculating the differential expression against a tree-based background for each gene in each sample then combine the log fold change and adjusted P− value to a gene score;
calculating a network score for each TF by performing a weighted sum of gene scores over at least one subnetwork centered on each TF;
ranking the TFs based on a combination of gene and network scores;
calculating the set of transcription factors for a conversion between any two cell types based on comparisons of ranked lists from each cell type; and optionally
removing transcriptionally redundant TFs from the lists.

thereby determining the transcription factors required for conversion of a source cell type to a target cell type.

The present invention provides a method for determining the transcription factors required for conversion of a source cell to a cell exhibiting at least one characteristic of a target cell type, the method comprising the steps of:
collecting expression data for each gene (x) in each sample (s);
calculating the differential expression against a tree-based background for each gene in each sample then combine the log fold change (Lₓ^{s}) and adjusted P− value (Pₓ^{s}) to a gene score (Gₓ^{s}).
calculating a network score (Nₓ^{s}) for each TF (x) by performing a weighted sum of gene scores over two different sub networks centered on each TF;
ranking TFs based on a combination of Gₓ^{s} and Nₓ^{s} scores;
calculating the set of transcription factors for a conversion between any two cell types based on comparisons of ranked lists from each cell type.
removing transcriptionally redundant TFs from the lists.

thereby determining the transcription factors required for conversion of a source cell type to a target cell type.

Preferably, the set of transcription factors identified are those that influence expression of at least about 80%, 85%, 90%, 91%, 92%, 93%, 94%, 95%, 96%, 97%, 98% or 99% of genes expressed in the target cell type.

The source cell type and target cell type may be any cell type described in the FANTOM5 dataset, or any cell type described herein including Table 4.

Typically, the sub network is gene expression data to which MARA has been applied or is the STRING database (referred to herein as (Nₓ_{^{MARA}}^{s} and Nₓ_{^{STRING}}^{s})), although any sub network as referred to herein which contains information relating to the interactions of a transcription factor that affect gene expression may be used.

Preferably, the method further comprises the step of creating a cell conversion landscape by arranging the cell types on a 2D plane based on their required TFs and adding a height based on the average coverage of the required genes that are directly regulated by the TFs selected.

Preferably, any method described herein further comprises the step of creating a cell conversion landscape by arranging the cell types on a 2D plane based on their required TFs and add a height based on the average coverage of the required genes that are directly regulated by the TFs selected.

In any method of the invention described above, the method further comprises the step of increasing the amount of the transcription factors, determined as being required for conversion of a source cell type to a target cell type, in the source cell type.

The present invention provides a method for identifying an agent useful for promoting the conversion of a source cell type to a target cell type, the method comprising the steps of:
determining one or more transcription factors required for conversion of a source cell type to a target cell type by any method described herein;
screening one or more candidate agents for the ability to increase the amount of the one or more transcription factors required for conversion of a source cell type to a target cell type;

wherein an agent that increases the amount of the one or more transcription factors is an agent useful for promoting the conversion of a source cell type to a target cell type.

Preferably, the candidate agent can be any compound which one wishes to test including, but not limited to, proteins (such as antibodies or fragments thereof or antibody mimetics), peptides, nucleic acids (including RNA, DNA, antisense oligonucleotide, peptide nucleic acids), carbohydrates, organic compounds, small molecules, natural products, library extracts, bodily fluids. The candidate compound may be part of a library, for example a collection of compounds containing variations or modifications.

The present invention provides a method for reprogramming a source cell, the method comprising increasing the protein expression of one or transcription factors, or variant thereof, in the source cell, wherein the source cell is reprogrammed to exhibit at least one characteristic of a target cell, wherein:
the source cell is selected from the group consisting of dermal fibroblasts, epidermal keratinocytes, embryonic stem cells, pluripotent stem cells, mesenchymal stem cells, monocytes or cardiac fibroblasts;
the target cell is selected from the group consisting of chondrocytes, hair follicles, CD4+ T cells, CD8+ T cells, NK-cells, haemopoeitic stem cells (HSC), mesenchymal stem cells (MSC), MSC of adipose, MSC of bone marrow, oligodendrocyte, oligodendrocyte precursor, skeletal muscle cell, smooth muscle cell, fetal cardiomyocyte, epithelial cells, endothelial cells, keratinocytes and astrocytes; and
the transcription factors are one or more of those listed in Table 4.

The present invention provides a method of generating a cell exhibiting at least one characteristic of a target cell from a source cell, the method comprising:
increasing the amount of one or more transcription factors, or variant thereof, in the source cell; and
culturing the source cell for a sufficient time and under conditions to allow differentiation to a target cell; thereby generating the cell exhibiting at least one characteristic of a target cell from a source cell, wherein:
the source cell is selected from the group consisting of dermal fibroblasts, epidermal keratinocytes, embryonic stem cells, pluripotent stem cells, mesenchymal stem cells, monocytes or cardiac fibroblasts;
the target cell is selected from the group consisting of chondrocytes, hair follicles, CD4+ T cells, CD8+ T cells, NK (natural killer)-cells, haemopoeitic stem cells (HSC), mesenchymal stem cells (MSC), MSC of adipose, MSC of bone marrow, oligodendrocyte, oligodendrocyte precursor, skeletal muscle cell, smooth muscle cell, fetal cardiomyocytes, epithelial cells, endothelial cells, keratinocytes and astrocytes; and
the transcription factors are one or more of those listed in Table 4.

The present invention also provides a method for reprogramming a source cell listed in Table 4, the method comprising increasing the protein expression of the transcription factors in Table 4, or variants thereof, in the source cell, wherein the source is reprogrammed to exhibit at least one characteristic of a target cell.

The present invention provides a method for reprogramming a source cell to a cell that exhibits at least one characteristic of a target cell comprising: i) providing a source cell, or a cell population comprising a source cell; ii) transfecting said source cell with one or more nucleic acids comprising a nucleotide sequence that encodes one or more transcription factors; and iii) culturing said cell or cell population, and optionally monitoring the cell or cell population for at least one characteristic of the target cell, wherein:
the source cell is selected from the group consisting of dermal fibroblasts, epidermal keratinocytes, embryonic stem cells, pluripotent stem cells, mesenchymal stem cells, monocytes or cardiac fibroblasts;
the target cell is selected from the group consisting of chondrocytes, hair follicles, CD4+ T cells, CD8+ T cells, NK-cells, haemopoeitic stem cells (HSC), mesenchymal stem cells (MSC), MSC of adipose, MSC of bone marrow, oligodendrocyte, oligodendrocyte precursor, skeletal muscle cell, smooth muscle cell fetal cardiomyocytes, epithelial cells, endothelial cells, keratinocytes and astrocytes; and
the transcription factors are one or more of those listed in Table 4.

In any method of the invention described herein, the source cell is a fibroblast, and

(a) the target cell is a chondrocyte cell and the transcription factors are any one or more of BARX1, PITX1, SMAD6, FOXC1, SIX2 and AHR;

(b) the target cell is a hair follicle and the transcription factors are any one or more of ZIC1, PRRX2, RARB, VDR, FOXD1 and CREB3;

(c) the target cell is a CD4+ T cell and the transcription factors are any one or more of RORA, LEF1, JUN, FOS and BACH2;

(d) the target cell is a CD8+ T cell and the transcription factors are any one or more of RORA, FOS, SMAD7, JUN and RUNX3;

(e) the target cell is an NK cell and the transcription factors are any one or more of RORA, SMAD7, FOS, JUN and NFATC2;

(f) the target cell is a HSC and the transcription factors are any one or more of MYB, GATA1, GFI1 and GFI1B;

(g) the target cell is a MSC of adipose and the transcription factors are any one or more of NOTCH3, HIC1, ID1, ESRRA, IR1, SIX5, SREBF1 and SNAI2;

(h) the target cell is a MSC of bone marrow and the transcription factors are any one or more of SIX1, ID1, HOXA7, FOXC2, HOXA9, MAFB and IRX5;

(i) the target cell is a oligodendrocyte precursor and the transcription factors are any one or more of NKX2-1, ANKRD1, FOXA2, CDH1, ZFP42, IGF1, ICAM1 and FOS;

(j) the target cell is a skeletal muscle cell and the transcription factors are MYOG, HIC1 MYOD1, FOXD1, PITX3, SIX2, HOXA7 and JUNB;

(k) the target cell is a smooth muscle cell and the transcription factors are any one or more of GATA6, LIF, JUNB, CREB3, MEIS1 and PBX1;

(l) the target cell is a fetal cardiomyocyte and the transcription factors are any one or more of BMP10, GATA6, TBX5, FHL2, NKX2-5, HAND2, GATA4 and PPARGC1A;

(m) the target cell is an astrocyte and the transcription factors are any one or more of SOX2, SOX9, ARNT2, E2F5, PBX1, SMAD1, and RUNX2.

(n) the target cell is an epithelial cell and the transcription factors are any one or more of FOS, DBP, HES1, FOXA2, ESRRA, CDH1, FOXQ1 and PAX6;

(o) the target cell is an endothelial cell and the transcription factors are any one or more of SOX17, SMAD1, TAL1, IRF1, TCF7L1, MXD4 and JUNB; or

(p) the target cell is a keratinocyte and the transcription factors are any one or more of FOXQ1, SOX9, MAFB, CDH1, FOS, and REL.

In (a) to (p) immediately above, all of the transcription factors listed may be used. Preferably, the fibroblast is a dermal fibroblast.

In any method of the invention described herein, the source cell is a keratinocyte, and (a) the target cell is a chondrocyte cell and the transcription factors are any one or more of BARX1, PITX1, SMAD6, TGFB3, FOXC1 and SIX2;

(b) the target cell is a hair follicle and the transcription factors are any one or more of RUNX1T1, ZIC1, PRRX1, MSX1, EBF1, FOXD1 and RUNX2;

(c) the target cell is a CD4+ T cell and the transcription factors are any one or more of RORA, LEF1, JUN, FOS and NR3C1;

(d) the target cell is a CD8+ T cell and the transcription factors are any one or more of RORA, FOS, SMAD7, JUN and RUNX3;

(e) the target cell is an NK cell and the transcription factors are any one or more of RORA, SMAD7, FOS, JUN, NFATC2 and RUNX3;

(f) the target cell is a HSC and the transcription factors are any one or more of MYB, GATA1, GFI1 and GFI1B;

(g) the target cell is a MSC of adipose and the transcription factors are any one or more of TWIST1, HIC1, ID1, MSX1, IRF1, HOXB7, SNAI2 and E2F1;

(h) the target cell is a MSC of bone marrow and the transcription factors are any one or more of SIX1, TWSIT1, ID1, HMOX1, FOXC2 and HOXA7;

(i) the target cell is a oligodendrocyte precursor cell and the transcription factors are any one or more of NKX2-1, ANKRD1, ZFP42, FOS, IGF1, ICAM1, FOXA2 and CDH1;

(j) the target cell is a skeletal muscle cell and the transcription factors are any one or more of MYOG, MYOD1, RF1, PITX3, HOXA7, FOXD1 and SOX8;

(k) the target cell is a smooth muscle cell and the transcription factors are any one or more of IRF1, GATA6, LIF and MEIS1;

(l) the target cell is an endothelial cell and the transcription factors are any one or more of SOX17, TAL1, SMAD1, IRF1 and TCF7L1. or

(m) the target cell is an epithelial cell and the transcription factors are any one or more of NOTCH1, HR, DBP, OTX1, ESRRA, FOXQ1, PAX6, and IRX5.

In (a) to (m) immediately above, all of the transcription factors listed may be used. Preferably the keratinocyte is an epidermal keratinocyte. More preferably, the keratinocyte is an oral mucosa keratinocyte. More preferably, where the source cell is an oral mucosa keratinocyte, the target cell is a corneal epithelial cell.

In any method of the invention described herein, the source cell is an embryonic stem cell, and

(a) the target cell is a chondrocyte cell and the transcription factors are any one or more of BARX1, PITX1, SMAD6 and NFKB1;

(b) the target cell is a hair follicle and the transcription factors are any one or more of TWIST1, ZIC1, NR2F2, PRRX1, NFKB1 and AHR;

(c) the target cell is a CD4+ T cell and the transcription factors are any one or more of RORA, LEF1, JUN, FOS and BACH2;

(d) the target cell is a CD8+ T cell and the transcription factors are any one or more of RORA, FOS, SMAD7 and JUN;

(e) the target cell is an NK cell and the transcription factors are any one or more of RORA, SMAD7, FOS, JUN and NFATC2;

(f) the target cell is a HSC and the transcription factors are any one or more of MYB, IL1B, KLF1, GATA1, GFI1, GFI1B and NFE2;

(g) the target cell is a MSC of adipose and the transcription factors are any one or more of TWIST1, SNAI2, IRF1, MXD4, NFKB1, MSX1, HOXB7 and ESRRA;

(h) the target cell is a MSC of bone marrow and the transcription factors are any one or more of IRF1, RUNX1, CEBPB, AHR, FOXC2 and HOXA9;

(i) the target cell is a oligodendrocyte precursor cell and the transcription factors are any one or more of NKX2-1, ANKRD1, FOXA2, LMO3, FOS, IGF1, ICAM1 and CDH1;

(j) the target cell is a skeletal muscle cell and the transcription factors are any one or more of MYOG, IRF1, MYOD1, FOXD1, NFKB1, JUNB and HOXA7;

(k) the target cell is a smooth muscle cell and the transcription factors are any one or more of IRF1, NFKB1, JUNB, FOSL2, GATA6 and MEIS1;

(l) the target cell is an endothelial cell and the transcription factors are any one or more of SOX17, TAL1, SMAD1, HOXB7, JUNB. IRF1 and NFKB1;

(m) the target cell is an astrocyte and the transcription factors are any one or more of IRF1, SOX9, ARNT2, PAX6, SNAI2, SOX5, and RUNX2;

(n) the target cell is a keratinocyte and the transcription factors are any one or more of SOX9, NFKB1, MYC, NR2F2, AHR, FOSL1 and FOSL2 or

(o) the target cell is an epithelial cell and the transcription factors are any one or more of MYC, IL1B, FOS, NFKB1, ESRRA, FOXQ1, IRF1 and PAX6.

In (a) to (o) immediately above, all of the transcription factors listed may be used. Preferably, the embryonic stem cell is a human embryonic stem cell.

In any method of the invention described herein, the source cell is a monocyte cell and the target cell is a HSC and the transcription factors are any one or more of MYB, IL1B, GATA1, GFI1 and GFI1B. Preferably, all of the transcription factors listed are used.

In any method of the invention described herein, the source cell is a cardiac fibroblast cell and the target cell is a fetal cardiomyocyte and the transcription factors are any one or more of BMP10, GATA6, TBX5, ANKRD1, HAND1, PPARGC1A, NKX2-5 and GATA4. Preferably, all of the transcription factors listed are used.

In any method of the invention described herein, the source cell is a pluripotent cell and the target cell is an endothelial cell and the transcription factors are any one or more of SOX17, TAL1, HOXB7, NFKB1, IRF1, JUNB, and SMAD1. Preferably, all of the transcription factors listed are used. Preferably, the pluripotent cell is an induced pluripotent stem cell (iPSC).

In any method of the invention described herein, the source cell is a pluripotent cell and the target cell is an astrocyte and the transcription factors are any one or more of PAX6, POU3F2, SNAI2, RUNX2, SOX5, E2F5, and HMGB2. Preferably, all of the transcription factors listed are used. Preferably, the pluripotent cell is an induced pluripotent stem cell (iPSC).

In any method of the invention described herein, the source cell is a pluripotent cell and the target cell is a keratinocyte and the transcription factors are any one or more of TP63, TFAP2A, MYC, NFKBIA, SOX9, and NFKB1. Preferably, all of the transcription factors listed are used. Preferably, the pluripotent cell is an induced pluripotent stem cell (iPSC).

In any method of the invention described herein, the source cell is a bone marrow stem cell and the target cell is an astrocyte and the transcription factors are any one or more of SOX2, SOX9, ARNT2, MYBL2, POU3F2, E2F1 and HMGB2. Preferably, all of the transcription factors listed are used.

Preferably, the at least one characteristic of the target cell is up-regulation of any one or more target cell markers and/or change in cell morphology. Relevant markers are described herein and known to those in the art. Exemplary markers for the following target cells include:
Chondrocytes: CD49, CD10, CD9, CD95, Integrin α10β1,105 and production of sulphated glycosaminoglycans (GAG);
Hair follicles: CD200, PHLDA1 and follistatin;
CD4+ T-cell: CD3, CD4;
CD8+ T-cell: CD3, CD8;
NK-cell: CD56, CD2;
HSCs: CD45, CD19/20, CD14/15, CD34, CD90;
MSCs of adipose: CD13, CD29, CD90, CD105, CD10, CD45 and differentiation in vitro towards osteoblasts, adipocytes and chondrocytes;
MSCs of bone marrow: CD13, CD29, CD90, CD105, CD10, and differentiation in vitro towards osteoblasts, adipocytes and chondrocytes;
Oligodendrocytes and oligodendrocyte precursor; NG2 and PDGFRα QPCR for Olig2 and Nkx2.2;
skeletal muscle cell: MyoD, Myogenin and Desmin;
smooth muscle cell: Myocardin, Smooth Muscle Alpha Actin and Smooth muscle myosin heavy chain;—fetal cardiomyocytes: MEF2C, MYH6, ACTN1, CDH2 and GJA1;
endothelial cell: PeCAM (CD31), VE-cadherin and VEGFR2;
keratinocytes: keratin1, keratin14, Pan-keratin and involucrin;
astrocyte: GFAP, S100B and ALDH1L1; and
epithelial cells: cytokeratin 15 (CK15), cytokeratin 3 (CK3), involucrin and connexin 4.

Typically, conditions suitable for target cell differentiation include culturing the cells for a sufficient time and in a suitable medium. A sufficient time of culturing may be at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days. A suitable medium may be one shown in Table 9.

The present invention also provides a cell exhibiting at least one characteristic of a target cell produced by a method as described herein.

In any method described herein, the method may further include the step of expanding the cells exhibiting at least one characteristic of a target cell type to increase the proportion of cells in the population exhibiting at least one characteristic of a target cell type. The step of expanding the cells may be in culture for a sufficient time and under conditions for generating a population of cells as described below.

In any method described herein, the method may further include the step of administering the cells, or cell population including a cell, exhibiting at least one characteristic of a target cell type, to an individual.

The present invention also provides a population of cells, wherein at least 5% of cells exhibit at least one characteristic of a target cell and those cells are produced by a method as described herein. Preferably, at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% A or 100% of the cells in the population exhibit at least one characteristic of a target cell.

The present invention also relates to kits for producing a cell exhibiting at least one characteristic of a target cell as disclose herein. In some embodiments, a kit comprises one or more nucleic acids having one or more nucleic acid sequences encoding a transcription factor described herein or variant thereof. Preferably, the kit can be used to produce a cell exhibiting at least one characteristic of a target cell referred to in Table 4. Preferably, the kit can be used with a source cell referred to in Table 4. In some embodiments, the kit further comprises instructions for reprogramming a source cell to a cell exhibiting at least one characteristic of a target cell according to the methods as disclosed herein. Preferably, the present invention provides a kit when used in a method of the invention described herein.

The present invention relates to a composition comprising at least one target cell and at least one agent which increases the protein expression of one or more transcription factors in the target cell. Preferably, a target cell is one as described herein. Further, the transcription factor may be any one described herein. Preferably, the target cell and transcription factors are as described in Table 4.

The present invention provides a method for reprogramming a fibroblast cell, the method comprising increasing the protein expression of any one or more of FOXQ1, SOX9, MAFB, CDH1, FOS and REL, or variant thereof, in the fibroblast cell, wherein the fibroblast cell is reprogrammed to exhibit at least one characteristic of a keratinocyte cell.

The present invention provides a method of generating a cell exhibiting at least one characteristic of a keratinocyte cell from a fibroblast cell, the method comprising:
increasing the amount of any one or more of FOXQ1, SOX9, MAFB, CDH1, FOS and REL, or variant thereof, in the fibroblast cell; and
culturing the fibroblast cell for a sufficient time and under conditions for keratinocyte differentiation; thereby generating the cell exhibiting at least one characteristic of a keratinocyte cell from a fibroblast cell.

The present invention provides a method for reprogramming a fibroblast cell to a cell that exhibits at least one characteristic of a keratinocyte cell comprising: i) providing a fibroblast cell, or a cell population comprising a fibroblast cell; ii) transfecting said fibroblast cell with one or more nucleic acids comprising a nucleotide sequence that encodes the polypeptides FOXQ1, SOX9, MAFB, CDH1, FOS and REL; and iii) culturing said cell or cell population, and optionally monitoring the cell or cell population for at least one characteristic of a keratinocyte cell.

Preferably, the at least one characteristic of the keratinocyte cell is up-regulation of any one or more keratinocyte markers and/or change in cell morphology. Keratinocyte markers include keratin1, keratin14 and involucrin and the cell morphology is cobblestone appearance.

Typically, conditions suitable for keratinocyte differentiation include culturing the cells for a sufficient time and in a suitable medium. A sufficient time of culturing may be at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days. A suitable medium may be one shown in Table 9.

The present invention also provides a cell exhibiting at least one characteristic of a keratinocyte cell produced by a method as described herein.

In any method described herein, the method may further include the step of expanding the cells exhibiting at least one characteristic of a target cell type to increase the proportion of cells in the population exhibiting at least one characteristic of a target cell type. The step of expanding the cells may be in culture for a sufficient time and under conditions for generating a population of cells as described below.

In any method described herein, the method may further include the step of administering the cells, or cell population including a cell, exhibiting at least one characteristic of a keratinocyte cell, to an individual.

The present invention also provides a population of cells, wherein at least 5% of cells exhibit at least one characteristic of a keratinocyte cell and those cells are produced by a method as described herein. Preferably, at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% of the cells in the population exhibit at least one characteristic of a keratinocyte cell.

The present invention also relates to kits for producing a cell exhibiting at least one characteristic of a keratinocyte cell as disclose herein. In some embodiments, a kit comprises any one or more of (i) a nucleic acid sequence encoding a FOXQ1 polypeptide or variant thereof; and (ii) a nucleic acid sequence encoding a SOX9 polypeptide or variant thereof; and (iii) a nucleic acid sequence encoding a MAFB polypeptide or variant thereof, and (iv) a nucleic acid sequence encoding a CDH1 polypeptide or variant thereof, and (v) a nucleic acid sequence encoding a FOS polypeptide or variant thereof, and (vi) a nucleic acid sequence encoding a REL polypeptide or variant thereof. In some embodiments, the kit further comprises instructions for reprogramming a fibroblast cell to a cell exhibiting at least one characteristic of a keratinocyte cell according to the methods as disclosed herein.

Preferably, the present invention provides a kit when used in a method of the invention described herein.

The present invention relates to a composition comprising at least one fibroblast cell and at least one agent which increases the protein expression of any one or more of FOXQ1, SOX9, MAFB, CDH1, FOS and REL in the fibroblast cell.

The present invention provides a method for reprogramming a fibroblast cell, the method comprising increasing the protein expression of any one or more of SOX17, SMAD1, TAL1, IRF1, TCF7L1, MXD4 and JUNB, or variant thereof, in the fibroblast cell, wherein the fibroblast cell is reprogrammed to exhibit at least one characteristic of an endothelial cell.

The present invention provides a method of generating a cell exhibiting at least one characteristic of an endothelial cell from a fibroblast cell, the method comprising:
increasing the amount of any one or more of SOX17, SMAD1, TAL1, IRF1, TCF7L1, MXD4 and JUNB, or variant thereof, in the fibroblast cell; and
culturing the fibroblast cell for a sufficient time and under conditions for endothelial differentiation; thereby generating the cell exhibiting at least one characteristic of an endothelial cell from a fibroblast cell.

The present invention provides a method for reprogramming a fibroblast cell to a cell that exhibits at least one characteristic of an endothelial cell comprising: i) providing a fibroblast cell, or a cell population comprising a fibroblast cell; ii) transfecting said fibroblast cell with one or more nucleic acids comprising a nucleotide sequence that encodes the polypeptides SOX17, SMAD1, TAL1, IRF1, TCF7L1, MXD4 and JUNB; and iii) culturing said cell or cell population, and optionally monitoring the cell or cell population for at least one characteristic of an endothelial cell.

Preferably, the at least one characteristic of the endothelial cell is up-regulation of any one or more endothelial markers and/or change in cell morphology. Endothelial markers include CD31 (Pe-CAM), VE-Cadherin and VEGFR2 and the cell morphology may be a capillary-like structure.

Typically, conditions suitable for endothelial differentiation include culturing the cells for a sufficient time and in a suitable medium. A sufficient time of culturing may be at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days. A suitable medium may be one shown in Table 9.

The present invention also provides a cell exhibiting at least one characteristic of a endothelial cell produced by a method as described herein.

In any method described herein, the method may further include the step of administering the cells, or cell population including a cell, exhibiting at least one characteristic of an endothelial cell, to an individual.

The present invention also provides a population of cells, wherein at least 5% of cells exhibit at least one characteristic of an endothelial cell and those cells are produced by a method as described herein. Preferably, at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% of the cells in the population exhibit at least one characteristic of an endothelial cell.

The present invention also relates to kits for producing a cell exhibiting at least one characteristic of an endothelial cell as disclose herein. In some embodiments, a kit comprises any one or more of (i) a nucleic acid sequence encoding a SOX17 polypeptide or variant thereof; and (ii) a nucleic acid sequence encoding a SMAD1 polypeptide or variant thereof; and (iii) a nucleic acid sequence encoding a IRF1 polypeptide or variant thereof, (iv) a nucleic acid sequence encoding a TCF7L1 polypeptide or variant thereof, (v) a nucleic acid sequence encoding a MXD4 polypeptide or variant thereof, (vi) a nucleic acid sequence encoding a TAL1 polypeptide or variant thereof; and (vii) a nucleic acid sequence encoding a JUNB polypeptide or variant thereof. In some embodiments, the kit further comprises instructions for reprogramming a fibroblast cell to a cell exhibiting at least one characteristic of an endothelial cell according to the methods as disclosed herein. Preferably, the present invention provides a kit when used in a method of the invention described herein.

The present invention relates to a composition comprising at least one fibroblast cell and at least one agent which increases the protein expression of any one or more of SOX17, SMAD1, TAL1, IRF1, TCF7L1, MXD4 and JUNB in the fibroblast cell.

The present invention provides a method for reprogramming a fibroblast cell, the method comprising increasing the protein expression of any one or more of SOX2, SOX9, ARNT2, E2F5, PXB1, SMAD1, and RUNX2, or variant thereof, in the fibroblast cell, wherein the fibroblast cell is reprogrammed to exhibit at least one characteristic of an astrocyte.

The present invention provides a method of generating a cell exhibiting at least one characteristic of an astrocyte from a fibroblast cell, the method comprising:
increasing the amount of any one or more of SOX2, SOX9, ARNT2, E2F5, PXB1, SMAD1, and RUNX2 or variant thereof, in the fibroblast cell; and
culturing the fibroblast cell for a sufficient time and under conditions for astrocyte differentiation; thereby generating the cell exhibiting at least one characteristic of an astrocyte from a fibroblast cell.

The present invention provides a method for reprogramming a fibroblast cell to a cell that exhibits at least one characteristic of an astrocyte comprising: i) providing a fibroblast cell, or a cell population comprising a fibroblast cell; ii) transfecting said fibroblast cell with one or more nucleic acids comprising a nucleotide sequence that encodes the polypeptides SOX2, SOX9, ARNT2, E2F5, PXB1, SMAD1, and RUNX2; and iii) culturing said cell or cell population, and optionally monitoring the cell or cell population for at least one characteristic of an astrocyte.

Preferably, the at least one characteristic of the astrocyte cell is up-regulation of any one or more astrocyte markers and/or change in cell morphology. Astrocyte markers include GFAP, S100B, and ALDH1L1. Preferably, the marker used is GFAP. Preferably the observed morphology is the presence of star like projections. Typically, conditions suitable for astrocyte differentiation include culturing the cells for a sufficient time and in a suitable medium. A sufficient time of culturing may be at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days. A suitable medium may be one shown in Table 9.

The present invention also provides a cell exhibiting at least one characteristic of an astrocyte cell produced by a method as described herein.

In any method described herein, the method may further include the step of administering the cells, or cell population including a cell, exhibiting at least one characteristic of an astrocyte, to an individual.

The present invention also provides a population of cells, wherein at least 5% of cells exhibit at least one characteristic of an astrocyte and those cells are produced by a method as described herein. Preferably, at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% A or 100% of the cells in the population exhibit at least one characteristic of an astrocyte.

The present invention also relates to kits for producing a cell exhibiting at least one characteristic of an astrocyte as disclose herein. In some embodiments, a kit comprises any one or more of (i) a nucleic acid sequence encoding a SOX2 polypeptide or variant thereof; and (ii) a nucleic acid sequence encoding a SOX9 polypeptide or variant thereof; (iii) a nucleic acid sequence encoding a ARNT2 polypeptide or variant thereof, (iv) a nucleic acid sequence encoding a E2F5 polypeptide or variant thereof, (v) a nucleic acid sequence encoding a PXB1 polypeptide or variant thereof; (vi) a nucleic acid sequence encoding a SMAD1 polypeptide or variant thereof, and (vii) a nucleic acid sequence encoding a RUNX2 polypeptide or variant thereof. In some embodiments, the kit further comprises instructions for reprogramming a fibroblast cell to a cell exhibiting at least one characteristic of an astrocyte according to the methods as disclosed herein. Preferably, the present invention provides a kit when used in a method of the invention described herein.

The present invention relates to a composition comprising at least one fibroblast cell and at least one agent which increases the protein expression of any one or more of SOX2, SOX9, ARNT2, E2F5, PXB1, SMAD1, and RUNX2 in the fibroblast cell.

The present invention provides a method for reprogramming a fibroblast cell, the method comprising increasing the protein expression of any one or more of FOS, DBP, HES1, FOXA2, ESRRA, CDH1, FOXQ1 and PAX6 or variant thereof, in the fibroblast cell, wherein the fibroblast cell is reprogrammed to exhibit at least one characteristic of an epithelial cell.

The present invention provides a method of generating a cell exhibiting at least one characteristic of an epithelial cell from a fibroblast cell, the method comprising:
increasing the amount of any one or more of FOS, DBP, HES1, FOXA2, ESRRA, CDH1, FOXQ1 and PAX6 or variant thereof, in the fibroblast cell; and
culturing the fibroblast cell for a sufficient time and under conditions for epithelial differentiation; thereby generating the cell exhibiting at least one characteristic of an epithelial cell from a fibroblast cell.

The present invention provides a method for reprogramming a fibroblast cell to a cell that exhibits at least one characteristic of an epithelial cell comprising: i) providing a fibroblast cell, or a cell population comprising a fibroblast cell; ii) transfecting said fibroblast cell with one or more nucleic acids comprising a nucleotide sequence that encodes the polypeptides FOS, DBP, HES1, FOXA2, ESRRA, CDH1, FOXQ1 and PAX6; and iii) culturing said cell or cell population, and optionally monitoring the cell or cell population for at least one characteristic of an epithelial cell.

Preferably, the at least one characteristic of the epithelial cell is up-regulation of any one or more epithelial markers and/or change in cell morphology. Epithelial markers include cytokeratin 15 (CK15), cytokeratin 3 (CK3), involucrin and connexin 4. Preferably the observed morphology is a cobblestone appearance.

Typically, conditions suitable for epithelial differentiation include culturing the cells for a sufficient time and in a suitable medium. A sufficient time of culturing may be at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days. A suitable medium may be one shown in Table 9.

The present invention also provides a cell exhibiting at least one characteristic of an epithelial cell produced by a method as described herein.

In any method described herein, the method may further include the step of administering the cells, or cell population including a cell, exhibiting at least one characteristic of an epithelial cell, to an individual.

The present invention also provides a population of cells, wherein at least 5% of cells exhibit at least one characteristic of an epithelial cell and those cells are produced by a method as described herein. Preferably, at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% of the cells in the population exhibit at least one characteristic of an epithelial cell.

The present invention also relates to kits for producing a cell exhibiting at least one characteristic of an epithelial cell as disclose herein. In some embodiments, a kit comprises any one or more of (i) a nucleic acid sequence encoding a FOS polypeptide or variant thereof; and (ii) a nucleic acid sequence encoding a DBP polypeptide or variant thereof; (iii) a nucleic acid sequence encoding a FOXA2 polypeptide or variant thereof, (iv) a nucleic acid sequence encoding a ESRRA polypeptide or variant thereof, (v) a nucleic acid sequence encoding a CDH1 polypeptide or variant thereof; (vi) a nucleic acid sequence encoding a FOXQ1 polypeptide or variant thereof, and (vii) a nucleic acid sequence encoding a PAX6 polypeptide or variant thereof. In some embodiments, the kit further comprises instructions for reprogramming a fibroblast cell to a cell exhibiting at least one characteristic of an epithelial cell according to the methods as disclosed herein. Preferably, the present invention provides a kit when used in a method of the invention described herein.

The present invention relates to a composition comprising at least one fibroblast cell and at least one agent which increases the protein expression of any one or more of FOS, DBP, HES1, FOXA2, ESRRA, CDH1, FOXQ1 and PAX6 in the fibroblast cell.

The present invention provides a method for reprogramming a keratinocyte cell, the method comprising increasing the protein expression of any one or more of SOX17, TAL1, SMAD1, IRF1, HOXB7 and TCF7L1 in the keratinocyte cell, wherein the keratinocyte cell is reprogrammed to exhibit at least one characteristic of an endothelial cell.

The present invention provides a method of generating a cell exhibiting at least one characteristic of an endothelial cell from a keratinocyte cell, the method comprising:

increasing the amount of any one or more of SOX17, TAL1, SMAD1, IRF1, HOXB7 and TCF7L1, or variant thereof, in the keratinocyte cell; and

culturing the keratinocyte cell for a sufficient time and under conditions for endothelial differentiation; thereby generating the cell exhibiting at least one characteristic of an endothelial cell from a keratinocyte cell.

The present invention provides a method for reprogramming a keratinocyte cell to a cell that exhibits at least one characteristic of an endothelial cell comprising: i) providing a keratinocyte cell, or a cell population comprising a keratinocyte cell; ii) transfecting said keratinocyte cell with one or more nucleic acids comprising a nucleotide sequence that encodes the polypeptides SOX17, TAL1, SMAD1, IRF1, HOXB7 and TCF7L1; and iii) culturing said cell or cell population, and optionally monitoring the cell or cell population for at least one characteristic of an endothelial cell.

Preferably, in any aspect of the present invention the endothelial cell is a microvascular endothelial cell.

Preferably, the at least one characteristic of the endothelial cell is up-regulation of any one or more endothelial markers and/or change in cell morphology. Endothelial markers include CD31, VE-Cadherin and VEGFR2.

Typically, conditions suitable for endothelial differentiation include culturing the cells for a sufficient time and in a suitable medium. A sufficient time of culturing may be at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days. A suitable medium may be one shown in Table 9.

The present invention also provides a cell exhibiting at least one characteristic of a microvascular endothelial cells cell produced by a method as described herein.

The present invention also provides a population of cells, wherein at least 5% of cells exhibit at least one characteristic of an endothelial cell, preferably a microvascular endothelial cell, and those cells are produced by a method as described herein. Preferably, at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% of the cells in the population exhibit at least one characteristic of an endothelial cell, preferably a microvascular endothelial cell.

The present invention also relates to kits for producing a cell exhibiting at least one characteristic of an endothelial cell as disclose herein. In some embodiments, a kit comprises any one or more of (i) a nucleic acid sequence encoding a SOX17 polypeptide or variant thereof; and (ii) a nucleic acid sequence encoding a TAL1 polypeptide or variant thereof; and (iii) a nucleic acid sequence encoding a SMAD1 polypeptide or variant thereof, and (iv) a nucleic acid sequence encoding a IRF1 polypeptide or variant thereof, (v) a nucleic acid sequence encoding a TCF7L1 polypeptide or variant thereof; and (vi) a nucleic acid sequence encoding a HOXB7 polypeptide or variant thereof. In some embodiments, the kit further comprises instructions for reprogramming a keratinocyte cell to a cell exhibiting at least one characteristic of an endothelial cell according to the methods as disclosed herein. Preferably, the present invention provides a kit when used in a method of the invention described herein.

The present invention relates to a composition comprising at least one keratinocyte cell and at least one agent which increases the protein expression of any one or more of SOX17, TAL1, SMAD1, IRF1, HOXB7 and TCF7L1 in the keratinocyte cell.

The present invention provides a method for reprogramming a keratinocyte cell, the method comprising increasing the protein expression of any one or more of NOTCH1, HR, DBP, OTX1, ESRRA, FOXQ1, PAX6 and IRX5 in the keratinocyte cell, wherein the keratinocyte cell is reprogrammed to exhibit at least one characteristic of an epithelial cell.

The present invention provides a method of generating a cell exhibiting at least one characteristic of an epithelial cell from a keratinocyte cell, the method comprising:

increasing the amount of any one or more of NOTCH1, HR, DBP, OTX1, ESRRA, FOXQ1, PAX6 and IRX5 or variant thereof, in the keratinocyte cell; and

culturing the keratinocyte cell for a sufficient time and under conditions for epithelial differentiation; thereby generating the cell exhibiting at least one characteristic of an epithelial cell from a keratinocyte cell.

The present invention provides a method for reprogramming a keratinocyte cell to a cell that exhibits at least one characteristic of an epithelial cell comprising: i) providing a keratinocyte cell, or a cell population comprising a keratinocyte cell; ii) transfecting said keratinocyte cell with one or more nucleic acids comprising a nucleotide sequence that encodes the polypeptides NOTCH1, HR, DBP, OTX1, ESRRA, FOXQ1, PAX6 and IRX5; and iii) culturing said cell or cell population, and optionally monitoring the cell or cell population for at least one characteristic of an epithelial cell.

Preferably, in any aspect of the present invention the epithelial cell is a corneal epithelial cell.

Preferably, the at least one characteristic of the epithelial cell is up-regulation of any one or more epithelial markers and/or change in cell morphology. Epithelial markers include cytokeratin 15 (CK15), cytokeratin 3 (CK3), involucrin and connexin 4.

Typically, conditions suitable for endothelial differentiation include culturing the cells for a sufficient time and in a suitable medium. A sufficient time of culturing may be at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days. A suitable medium may be one shown in Table 9.

The present invention also provides a cell exhibiting at least one characteristic of an epithelial cell, preferably a corneal epithelial cell produced by a method as described herein.

The present invention also provides a population of cells, wherein at least 5% of cells exhibit at least one characteristic of an epithelial cell, preferably a corneal epithelial cell, and those cells are produced by a method as described herein. Preferably, at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% of the cells in the population exhibit at least one characteristic of an epithelial cell, preferably a corneal epithelial cell.

The present invention also relates to kits for producing a cell exhibiting at least one characteristic of an epithelial cell as disclose herein. In some embodiments, a kit comprises any one or more of (i) a nucleic acid sequence encoding a NOTCH1 polypeptide or variant thereof; and (ii) a nucleic acid sequence encoding a HR polypeptide or variant thereof; and (iii) a nucleic acid sequence encoding a DBP polypeptide or variant thereof, and (iv) a nucleic acid sequence encoding a OTX1 polypeptide or variant thereof, (v) a nucleic acid sequence encoding a ESRRA polypeptide or variant thereof; (vi) a nucleic acid sequence encoding a FOXQ1 polypeptide or variant thereof; (vii) a nucleic acid sequence encoding a PAX6 polypeptide or variant thereof; and (viii) a nucleic acid sequence encoding a IRX5 polypeptide or variant thereof. In some embodiments, the kit further comprises instructions for reprogramming a keratinocyte cell to a cell exhibiting at least one characteristic of an epithelial cell according to the methods as disclosed herein. Preferably, the present invention provides a kit when used in a method of the invention described herein.

The present invention relates to a composition comprising at least one keratinocyte cell and at least one agent which increases the protein expression of any one or more of NOTCH1, HR, DBP, OTX1, ESRRA, FOXQ1, PAX6 and IRX5 in the keratinocyte cell.

The present invention provides a method for differentiating an embryonic stem cell, the method comprising increasing the protein expression of any one or more of SOX17, TAL1, SMAD1, HOXB7, JUNB, IRF1 and NFKB1 in the embryonic stem cell, wherein the embryonic stem cell is differentiated to exhibit at least one characteristic of an endothelial cell.

The present invention provides a method of generating a cell exhibiting at least one characteristic of an endothelial cell from an embryonic stem cell, the method comprising:

increasing the amount of any one or more of SOX17, TAL1, SMAD1, HOXB7, JUNB, IRF1 and NFKB1, or variant thereof, in the embryonic stem cell; and

culturing the embryonic stem cell for a sufficient time and under conditions for endothelial differentiation; thereby generating the cell exhibiting at least one characteristic of an endothelial cell from an embryonic stem cell.

The present invention provides a method for differentiating an embryonic stem cell to a cell that exhibits at least one characteristic of an endothelial cell comprising: i) providing an embryonic stem cell, or a cell population comprising an embryonic stem cell; ii) transfecting said embryonic stem cell with one or more nucleic acids comprising a nucleotide sequence that encodes any one or more of the polypeptides SOX17, TAL1, SMAD1, HOXB7, JUNB, IRF1 and NFKB1; and iii) culturing said cell or cell population, and optionally monitoring the cell or cell population for at least one characteristic of an endothelial cell.

Preferably, in any aspect of the present invention the endothelial cell is a microvascular endothelial cell.

Preferably, the at least one characteristic of the endothelial cell is up-regulation of any one or more endothelial markers and/or change in cell morphology. Endothelial markers include CD31, VE-Cadherin and VEGFR2.

Typically, conditions suitable for endothelial differentiation include culturing the cells for a sufficient time and in a suitable medium. A sufficient time of culturing may be at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days. A suitable medium may be one shown in Table 9.

The present invention also provides a cell exhibiting at least one characteristic of a microvascular endothelial cell produced by a method as described herein.

The present invention also provides a population of cells, wherein at least 5% of cells exhibit at least one characteristic of an endothelial cell, preferably a microvascular endothelial cell, and those cells are produced by a method as described herein. Preferably, at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% of the cells in the population exhibit at least one characteristic of an endothelial cell, preferably a microvascular endothelial cell.

The present invention also relates to kits for producing a cell exhibiting at least one characteristic of an endothelial cell as disclose herein. In some embodiments, a kit comprises any one or more of (i) a nucleic acid sequence encoding a SOX17 polypeptide or variant thereof; (ii) a nucleic acid sequence encoding a TAL1 polypeptide or variant thereof; (iii) a nucleic acid sequence encoding a SMAD1 polypeptide or variant thereof, (iv) a nucleic acid sequence encoding a IRF1 polypeptide or variant thereof, (v) a nucleic acid sequence encoding a NFKB1 polypeptide or variant thereof; (vi) a nucleic acid sequence encoding a HOXB7 polypeptide or variant thereof; and (vii) a nucleic acid sequence encoding a JUNB polypeptide or variant thereof. In some embodiments, the kit further comprises instructions for differentiating an embryonic stem cell to a cell exhibiting at least one characteristic of an endothelial cell according to the methods as disclosed herein. Preferably, the present invention provides a kit when used in a method of the invention described herein.

The present invention relates to a composition comprising at least one embryonic stem cell and at least one agent which increases the protein expression of any one or more of SOX17, TAL1, SMAD1, HOXB7, JUNB, IRF1 and NFKB1 in the embryonic stem cell.

The present invention provides a method for differentiating an embryonic stem cell, the method comprising increasing the protein expression of IRF1, SOX9, ARNT2, PAX6, SNAI2, SOX5 and RUNX2 in the embryonic stem cell, wherein the embryonic stem cell is differentiated to exhibit at least one characteristic of an astrocyte.

The present invention provides a method of producing or generating a cell exhibiting at least one characteristic of an astrocyte from an embryonic stem cell, the method comprising:

increasing the amount of any one or more of IRF1, SOX9, ARNT2, PAX6, SNAI2, SOX5 and RUNX2, or variant thereof, in the embryonic stem cell; and

culturing the embryonic stem cell for a sufficient time and under conditions for astrocyte differentiation; thereby generating the cell exhibiting at least one characteristic of an astrocyte from an embryonic stem cell.

The present invention provides a method for differentiating an embryonic stem cell to a cell that exhibits at least one characteristic of an astrocyte comprising: i) providing an embryonic stem cell, or a cell population comprising an embryonic stem cell; ii) transfecting said embryonic stem cell with one or more nucleic acids comprising a nucleotide sequence that encodes any one or more of the polypeptides of IRF1, SOX9, ARNT2, PAX6, SNAI2, SOX5 and RUNX2; and iii) culturing said cell or cell population, and optionally monitoring the cell or cell population for at least one characteristic of an astrocyte.

Preferably, the at least one characteristic of the astrocyte is up-regulation of any one or more astrocyte markers and/or change in cell morphology. Astrocyte markers include GFAP, S100B, and ALDH1L1. Preferably, the marker used is GFAP. Preferably the observed morphology is the presence of star like projections.

Typically, conditions suitable for astrocyte differentiation include culturing the cells for a sufficient time and in a suitable medium. A sufficient time of culturing may be at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days. A suitable medium may be one shown in Table 9.

The present invention also provides a cell exhibiting at least one characteristic of an astrocyte produced by a method as described herein.

The present invention also provides a population of cells, wherein at least 5% of cells exhibit at least one characteristic of an astrocyte, and those cells are produced by a method as described herein. Preferably, at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% A or 100% of the cells in the population exhibit at least one characteristic of an astrocyte.

The present invention also relates to kits for producing a cell exhibiting at least one characteristic of an astrocyte as disclose herein. In some embodiments, a kit comprises any one or more of (i) a nucleic acid sequence encoding a IRF1 polypeptide or variant thereof; (ii) a nucleic acid sequence encoding a SOX9 polypeptide or variant thereof; (iii) a nucleic acid sequence encoding a ARNT2 polypeptide or variant thereof, (iv) a nucleic acid sequence encoding a PAX6 polypeptide or variant thereof, (v) a nucleic acid sequence encoding a SNAI2 polypeptide or variant thereof, (vi) a nucleic acid sequence encoding a RUNX2 polypeptide or variant thereof; and (vii) a nucleic acid sequence encoding a SOX5 polypeptide or variant thereof. In some embodiments, the kit further comprises instructions for differentiating an embryonic stem cell to a cell exhibiting at least one characteristic of an astrocyte cell according to the methods as disclosed herein. Preferably, the present invention provides a kit when used in a method of the invention described herein.

The present invention relates to a composition comprising at least one embryonic stem cell and at least one agent which increases the protein expression of IRF1, SOX9, ARNT2, PAX6, SNAI2, SOX5 and RUNX2 in the embryonic stem cell.

The present invention provides a method for differentiating an embryonic stem cell, the method comprising increasing the protein expression of any one or more of SOX9, NFKB1, MYC, NR2F2, FOSL1, AHR and FOSL2 in the embryonic stem cell, wherein the embryonic stem cell is differentiated to exhibit at least one characteristic of a keratinocyte.

The present invention provides a method of generating a cell exhibiting at least one characteristic of a keratinocyte from an embryonic stem cell, the method comprising:

increasing the amount of any one or more of SOX9, NFKB1, MYC, NR2F2, FOSL1, AHR and FOSL2, or variant thereof, in the embryonic stem cell; and

culturing the embryonic stem cell for a sufficient time and under conditions for a keratinocyte differentiation; thereby generating the cell exhibiting at least one characteristic of a keratinocyte from an embryonic stem cell.

The present invention provides a method for differentiation of an embryonic stem cell to a cell that exhibits at least one characteristic of a keratinocyte comprising: i) providing an embryonic stem cell, or a cell population comprising an embryonic stem cell; ii) transfecting said embryonic stem cell with one or more nucleic acids comprising a nucleotide sequence that encodes any one or more of the polypeptides of SOX9, NFKB1, MYC, NR2F2, FOSL1, AHR and FOSL2; and iii) culturing said cell or cell population, and optionally monitoring the cell or cell population for at least one characteristic of a keratinocyte.

Preferably, the at least one characteristic of the keratinocyte is up-regulation of any one or more keratinocyte markers and/or change in cell morphology. Keratinocyte markers include pan-Keratin, keratin 1, keratin 14 and involucrin and the cell morphology is cobblestone appearance.

Typically, conditions suitable for keratinocyte differentiation include culturing the cells for a sufficient time and in a suitable medium. A sufficient time of culturing may be at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days. A suitable medium may be one shown in Table 9.

The present invention also provides a cell exhibiting at least one characteristic of a keratinocyte produced by a method as described herein.

The present invention also provides a population of cells, wherein at least 5% of cells exhibit at least one characteristic of a keratinocyte, and those cells are produced by a method as described herein. Preferably, at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% of the cells in the population exhibit at least one characteristic of a keratinocyte.

The present invention also relates to kits for producing a cell exhibiting at least one characteristic of a keratinocyte as disclose herein. In some embodiments, a kit comprises any one or more of (i) a nucleic acid sequence encoding a SOX9 polypeptide or variant thereof; (ii) a nucleic acid sequence encoding a NFKB1 polypeptide or variant thereof; (iii) a nucleic acid sequence encoding a MYC polypeptide or variant thereof, (iv) a nucleic acid sequence encoding a FOSL2 polypeptide or variant thereof; (v) a nucleic acid sequence encoding a NR2F2 polypeptide or variant thereof; (vi) a nucleic acid sequence encoding a FOSL1 polypeptide or variant thereof; and (vii) a nucleic acid sequence encoding a AHR polypeptide or variant thereof. In some embodiments, the kit further comprises instructions for differentiation of an embryonic stem cell to a cell exhibiting at least one characteristic of a keratinocyte according to the methods as disclosed herein. Preferably, the present invention provides a kit when used in a method of the invention described herein.

The present invention relates to a composition comprising at least one embryonic stem cell and at least one agent which increases the protein expression of SOX9, NFKB1, MYC, NR2F2, FOSL1, AHR and FOSL2 in the embryonic stem cell.

The present invention provides a method for differentiating an embryonic stem cell, the method comprising increasing the protein expression of any one or more of MYC, IL1B, FOS, NFKB1, ESRRA, FOXQ1, IRF1 and PAX6 in the embryonic stem cell, wherein the embryonic stem cell is differentiated to exhibit at least one characteristic of an epithelial cell, preferably a corneal epithelial cell.

The present invention provides a method of generating a cell exhibiting at least one characteristic of an epithelial cell from an embryonic stem cell, the method comprising:

increasing the amount of any one or more of MYC, IL1B, FOS, NFKB1, ESRRA, FOXQ1, IRF1 and PAX6, or variant thereof, in the embryonic stem cell; and

culturing the embryonic stem cell for a sufficient time and under conditions for a epithelial differentiation; thereby generating the cell exhibiting at least one characteristic of an epithelial cell from an embryonic stem cell.

The present invention provides a method for differentiation of an embryonic stem cell to a cell that exhibits at least one characteristic of an epithelial cell comprising: i) providing an embryonic stem cell, or a cell population comprising an embryonic stem cell; ii) transfecting said embryonic stem cell with one or more nucleic acids comprising a nucleotide sequence that encodes any one or more of the polypeptides of MYC, IL1B, FOS, NFKB1, ESRRA, FOXQ1, IRF1 and PAX6; and iii) culturing said cell or cell population, and optionally monitoring the cell or cell population for at least one characteristic of an epithelial cell.

Preferably, the at least one characteristic of the epithelial cell is up-regulation of any one or more epithelial markers and/or change in cell morphology. Epithelial markers include cytokeratin 15 (CK15), cytokeratin 3 (CK3), involucrin and connexin 4 and the cell morphology may be cobblestone appearance.

Typically, conditions suitable for epithelial differentiation include culturing the cells for a sufficient time and in a suitable medium. A sufficient time of culturing may be at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days. A suitable medium may be one shown in Table 9.

The present invention also provides a cell exhibiting at least one characteristic of an epithelial cell produced by a method as described herein.

The present invention also provides a population of cells, wherein at least 5% of cells exhibit at least one characteristic of an epithelial cell, and those cells are produced by a method as described herein. Preferably, at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% of the cells in the population exhibit at least one characteristic of an epithelial cell.

The present invention also relates to kits for producing a cell exhibiting at least one characteristic of an epithelial cell as disclose herein. In some embodiments, a kit comprises any one or more of (i) a nucleic acid sequence encoding a MYC polypeptide or variant thereof; (ii) a nucleic acid sequence encoding a IL1B polypeptide or variant thereof; (iii) a nucleic acid sequence encoding a FOS polypeptide or variant thereof, (iv) a nucleic acid sequence encoding a NFKB1 polypeptide or variant thereof; (v) a nucleic acid sequence encoding a ESRRA polypeptide or variant thereof; (vi) a nucleic acid sequence encoding a FOXQ1 polypeptide or variant thereof; (vii) a nucleic acid sequence encoding a IRF1 polypeptide or variant thereof; and (viii) a nucleic acid sequence encoding a PAX6 polypeptide or variant thereof. In some embodiments, the kit further comprises instructions for differentiation of an embryonic stem cell to a cell exhibiting at least one characteristic of an epithelial cell according to the methods as disclosed herein. Preferably, the present invention provides a kit when used in a method of the invention described herein.

The present invention relates to a composition comprising at least one embryonic stem cell and at least one agent which increases the protein expression of any one or more of MYC, IL1B, FOS, NFKB1, ESRRA, FOXQ1, IRF1 and PAX6 in the embryonic stem cell.

The present invention provides a method for producing an endothelial cell from a pluripotent stem cell, including differentiating a pluripotent stem cell, the method comprising increasing the protein expression of any one or more of SOX17, TAL1, NFKB1, IRF1, HOXB7, JUNB and SMAD1 in the pluripotent stem cell, wherein the pluripotent stem cell is differentiated to exhibit at least one characteristic of an endothelial cell.

In any aspect of the invention, including any method or composition, the pluripotent stem cell may be an induced pluripotent stem cell (iPSC).

The present invention provides a method of generating a cell exhibiting at least one characteristic of an endothelial cell from a pluripotent stem cell, the method comprising:

increasing the amount of any one or more of SOX17, TAL1, NFKB1, HOXB7, JUNB, IRF1 and SMAD1, or variant thereof, in the pluripotent stem cell; and

culturing the pluripotent stem cell for a sufficient time and under conditions for endothelial differentiation; thereby generating the cell exhibiting at least one characteristic of an endothelial cell from a pluripotent stem cell.

The present invention provides a method for differentiating a pluripotent stem cell to a cell that exhibits at least one characteristic of an endothelial cell comprising: i) providing a pluripotent stem cell, or a cell population comprising a pluripotent stem cell; ii) transfecting said pluripotent stem cell with one or more nucleic acids comprising a nucleotide sequence that encodes any one or more of the polypeptides SOX17, TAL1, NFKB1, HOXB7, JUNB, IRF1 and SMAD1, and iii) culturing said cell or cell population, and optionally monitoring the cell or cell population for at least one characteristic of an endothelial cell.

Preferably, the at least one characteristic of the endothelial cell is up-regulation of any one or more endothelial markers and/or change in cell morphology. Endothelial markers include pan-CD31, VE-Cadherin and VEGFR2.

Typically, conditions suitable for endothelial differentiation include culturing the cells for a sufficient time and in a suitable medium. A sufficient time of culturing may be at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days. A suitable medium may be one shown in Table 9.

The present invention also provides a cell exhibiting at least one characteristic of an endothelial cell produced by a method as described herein.

The present invention also provides a population of cells, wherein at least 5% of cells exhibit at least one characteristic of an endothelial cell, and those cells are produced by a method as described herein. Preferably, at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% A or 100% of the cells in the population exhibit at least one characteristic of an endothelial cell.

The present invention also relates to kits for producing a cell exhibiting at least one characteristic of an endothelial cell as disclose herein. In some embodiments, a kit comprises any one or more of (i) a nucleic acid sequence encoding a SOX17 polypeptide or variant thereof; (ii) a nucleic acid sequence encoding a TAL1 polypeptide or variant thereof; (iii) a nucleic acid sequence encoding a NFKB1 polypeptide or variant thereof, (iv) a nucleic acid sequence encoding a IRF1 polypeptide or variant thereof, (v) a nucleic acid sequence encoding a SMAD1 polypeptide or variant thereof; (vi) a nucleic acid sequence encoding a HOXB7 polypeptide or variant thereof; and (vii) a nucleic acid sequence encoding a JUNB polypeptide or variant thereof. In some embodiments, the kit further comprises instructions for differentiating a pluripotent stem cell to a cell exhibiting at least one characteristic of an endothelial cell according to the methods as disclosed herein. Preferably, the present invention provides a kit when used in a method of the invention described herein.

The present invention relates to a composition comprising at least one pluripotent stem cell and at least one agent which increases the protein expression of any one or more of SOX17, TAL1, NFKB1, IRF1, HOXB7, JUNB and SMAD1 in the pluripotent stem cell.

The present invention provides a method for producing an astrocyte from a pluripotent stem cell, including differentiating a pluripotent stem cell, the method comprising increasing the protein expression of any one or more of PAX6, SNAI2, POU3F2, SOX5, E2F5, RUNX2, and HMGB2 in the pluripotent stem cell, wherein the pluripotent stem cell is differentiated to exhibit at least one characteristic of an astrocyte.

The present invention provides a method of generating a cell exhibiting at least one characteristic of an astrocyte from a pluripotent stem cell, the method comprising:

increasing the amount of any one or more of PAX6, SNAI2, POU3F2, SOX5, E2F5, RUNX2, and HMGB2, or variant thereof, in the pluripotent stem cell; and

culturing the pluripotent stem cell for a sufficient time and under conditions for astrocyte differentiation; thereby generating the cell exhibiting at least one characteristic of an astrocyte from a pluripotent stem cell.

The present invention provides a method for differentiating a pluripotent stem cell to a cell that exhibits at least one characteristic of an astrocyte comprising: i) providing a pluripotent stem cell, or a cell population comprising a pluripotent stem cell; ii) transfecting said pluripotent stem cell with one or more nucleic acids comprising a nucleotide sequence that encodes any one or more of the polypeptides PAX6, SNAI2, POU3F2, SOX5, E2F5, RUNX2, and HMGB2 and iii) culturing said cell or cell population, and optionally monitoring the cell or cell population for at least one characteristic of an astrocyte.

Preferably, the at least one characteristic of the astrocyte is up-regulation of any one or more astrocyte markers and/or change in cell morphology. Astrocyte markers include GFAP, S100B, and ALDH1L1. Preferably, the marker used is GFAP. Preferably the observed morphology is the presence of star like projections.

Typically, conditions suitable for astrocyte differentiation include culturing the cells for a sufficient time and in a suitable medium. A sufficient time of culturing may be at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days. A suitable medium may be one shown in Table 9.

The present invention also provides a cell exhibiting at least one characteristic of an astrocyte produced by a method as described herein.

The present invention also provides a population of cells, wherein at least 5% of cells exhibit at least one characteristic of an astrocyte, and those cells are produced by a method as described herein. Preferably, at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% of the cells in the population exhibit at least one characteristic of an astrocyte.

The present invention also relates to kits for producing a cell exhibiting at least one characteristic of an astrocyte as disclose herein. In some embodiments, a kit comprises any one or more of (i) a nucleic acid sequence encoding a PAX6 polypeptide or variant thereof; (ii) a nucleic acid sequence encoding a SNAI2 polypeptide or variant thereof; (iii) a nucleic acid sequence encoding a RUNX2 polypeptide or variant thereof, (iv) a nucleic acid sequence encoding a HMGB2 polypeptide or variant thereof; (v) a nucleic acid sequence encoding a POU3F2 polypeptide or variant thereof; (vi) a nucleic acid sequence encoding a SOX5 polypeptide or variant thereof; and (vii) a nucleic acid sequence encoding a E2F5 polypeptide or variant thereof. In some embodiments, the kit further comprises instructions for differentiating a pluripotent stem cell to a cell exhibiting at least one characteristic of an astrocyte according to the methods as disclosed herein. Preferably, the present invention provides a kit when used in a method of the invention described herein.

The present invention relates to a composition comprising at least one pluripotent stem cell and at least one agent which increases the protein expression of any one or more of PAX6, SNAI2, POU3F2, SOX5, E2F5, RUNX2, and HMGB2 in the pluripotent stem cell.

The present invention provides a method for producing a keratinocyte from a pluripotent stem cell, including differentiating a pluripotent stem cell, the method comprising increasing the protein expression of any one or more of TFAP2A, MYC, SOX9, TP63, NFKBIA and NFKB1 in the pluripotent stem cell, wherein the pluripotent stem cell is differentiated to exhibit at least one characteristic of a keratinocyte.

The present invention provides a method of generating a cell exhibiting at least one characteristic of a keratinocyte from a pluripotent stem cell, the method comprising:

increasing the amount of any one or more TFAP2A, MYC, SOX9, TP63, NFKBIA and NFKB1 or variant thereof, in the pluripotent stem cell; and

culturing the pluripotent stem cell for a sufficient time and under conditions for keratinocyte differentiation; thereby generating the cell exhibiting at least one characteristic of a keratinocyte from a pluripotent stem cell.

The present invention provides a method for differentiating a pluripotent stem cell to a cell that exhibits at least one characteristic of a keratinocyte comprising: i) providing a pluripotent stem cell, or a cell population comprising a pluripotent stem cell; ii) transfecting said pluripotent stem cell with one or more nucleic acids comprising a nucleotide sequence that encodes any one or more of the polypeptides TFAP2A, MYC, SOX9, TP63, NFKBIA and NFKB1 and iii) culturing said cell or cell population, and optionally monitoring the cell or cell population for at least one characteristic of a keratinocyte.

Preferably, the at least one characteristic of the keratinocyte is up-regulation of any one or more keratinocyte markers and/or change in cell morphology. Keratinocyte markers include keratin1, keratin14 and involucrin and the cell morphology is cobblestone appearance.

Typically, conditions suitable for keratinocyte differentiation include culturing the cells for a sufficient time and in a suitable medium. A sufficient time of culturing may be at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days. A suitable medium may be one shown in Table 9.

The present invention also provides a cell exhibiting at least one characteristic of a keratinocyte produced by a method as described herein.

The present invention also provides a population of cells, wherein at least 5% of cells exhibit at least one characteristic of a keratinocyte, and those cells are produced by a method as described herein. Preferably, at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% of the cells in the population exhibit at least one characteristic of a keratinocyte.

The present invention also relates to kits for producing a cell exhibiting at least one characteristic of a keratinocyte as disclose herein. In some embodiments, a kit comprises any one or more of (i) a nucleic acid sequence encoding a TFAP2A polypeptide or variant thereof; (ii) a nucleic acid sequence encoding a MYC polypeptide or variant thereof; (iii) a nucleic acid sequence encoding a SOX9 polypeptide or variant thereof, (iv) a nucleic acid sequence encoding a NFKB1 polypeptide or variant thereof; (v) a nucleic acid sequence encoding a TP63 polypeptide or variant thereof; (vi) a nucleic acid sequence encoding a NFKBIA polypeptide or variant thereof. In some embodiments, the kit further comprises instructions for differentiating a pluripotent stem cell to a cell exhibiting at least one characteristic of a keratinocyte according to the methods as disclosed herein. Preferably, the present invention provides a kit when used in a method of the invention described herein.

The present invention relates to a composition comprising at least one pluripotent stem cell and at least one agent which increases the protein expression of any one or more of TFAP2A, MYC, SOX9, TP63, NFKBIA and NFKB1 in the pluripotent stem cell.

The present invention provides a method for producing an astrocyte from a bone marrow stem cell, including differentiating a bone marrow stem cell, the method comprising increasing the protein expression of any one or more of SOX2, SOX9, ARNT2, MYBL2, POU3F2, E2F1 and HMGB2 in the bone marrow stem cell, wherein the bone marrow stem cell is differentiated to exhibit at least one characteristic of an astrocyte.

The present invention provides a method of generating a cell exhibiting at least one characteristic of an astrocyte from a bone marrow stem cell, the method comprising:

increasing the amount of any one or more SOX2, SOX9, ARNT2, MYBL2, POU3F2, E2F1 and HMGB2 or variant thereof, in the bone marrow stem cell; and

culturing the bone marrow stem cell for a sufficient time and under conditions for astrocyte differentiation; thereby generating the cell exhibiting at least one characteristic of an astrocyte from a bone marrow stem cell.

The present invention provides a method for differentiating a bone marrow stem cell to a cell that exhibits at least one characteristic of an astrocyte comprising: i) providing a bone marrow stem cell, or a cell population comprising a bone marrow stem cell; ii) transfecting said bone marrow stem cell with one or more nucleic acids comprising a nucleotide sequence that encodes any one or more of the polypeptides SOX2, SOX9, ARNT2, MYBL2, POU3F2, E2F1 and HMGB2 and iii) culturing said cell or cell population, and optionally monitoring the cell or cell population for at least one characteristic of an astrocyte.

Preferably, the at least one characteristic of the astrocyte is up-regulation of any one or more astrocyte markers and/or change in cell morphology. Astrocyte markers include GFAP, S100B, and ALDH1L1. Preferably, the marker used is GFAP. Preferably the observed morphology is the presence of star like projections.

Typically, conditions suitable for astrocyte differentiation include culturing the cells for a sufficient time and in a suitable medium. A sufficient time of culturing may be at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days. A suitable medium may be one shown in Table 9.

The present invention also provides a cell exhibiting at least one characteristic of an astrocyte produced by a method as described herein.

The present invention also provides a population of cells, wherein at least 5% of cells exhibit at least one characteristic of an astrocyte, and those cells are produced by a method as described herein. Preferably, at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% of the cells in the population exhibit at least one characteristic of an astrocyte.

The present invention also relates to kits for producing a cell exhibiting at least one characteristic of an astrocyte as disclose herein. In some embodiments, a kit comprises any one or more of (i) a nucleic acid sequence encoding a SOX2 polypeptide or variant thereof; (ii) a nucleic acid sequence encoding a SOX9 polypeptide or variant thereof; (iii) a nucleic acid sequence encoding a ARNT2 polypeptide or variant thereof, (iv) a nucleic acid sequence encoding a E2F1 polypeptide or variant thereof; (v) a nucleic acid sequence encoding a HMGB2 polypeptide or variant thereof; (vi) a nucleic acid sequence encoding a POU3F2 polypeptide or variant thereof. In some embodiments, the kit further comprises instructions for differentiating a bone marrow stem cell to a cell exhibiting at least one characteristic of an astrocyte according to the methods as disclosed herein. Preferably, the present invention provides a kit when used in a method of the invention described herein.

The present invention relates to a composition comprising at least one bone marrow stem cell and at least one agent which increases the protein expression of any one or more of SOX2, SOX9, ARNT2, MYBL2, E2F1, POU3F2 and HMGB2 in the bone marrow stem cell.

Typically, the protein expression, or amount, of a transcription factor as described herein is increased by contacting the cell with an agent which increases the expression of the transcription factor. Preferably, the agent is selected from the group consisting of: a nucleotide sequence, a protein, an aptamer and small molecule, ribosome, RNAi agent and peptide-nucleic acid (PNA) and analogues or variants thereof. Preferably, the agent is exogenous.

Typically, the protein expression, or amount, of a transcription factor as described herein is increased by introducing at least one nucleic acid comprising a nucleotide sequence encoding a transcription factor, or encoding a functional fragment thereof, in the cell. Preferably, the nucleotide sequence encoding a transcription factor is at least 70%, 80%, 85%, 90%, 91%, 92%, 93%, 94%, 95%, 96%, 97%, 98%, or 99% A identical to a sequence with an accession number listed in Table 3.

Preferably, the nucleic acid further includes a heterologous promoter. Preferably, the nucleic acid is in a vector, such as a viral vector or a non-viral vector. Preferably, the vector is a viral vector comprising a genome that does not integrate into the host cell genome. The viral vector may be a retroviral vector or a lentiviral vector.

Any method as described herein may have one or more, or all, steps performed in vitro, ex vivo or in vivo.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. The Mogrify algorithm for predicting TFs for cell conversion. This is done as follows: (A) Mogrify aims to find those TFs that not only are differentially expressed but appear to be responsible for the regulation of many differentially expressed genes in a given cell type. (B) We use the cell type ontology tree created as part of the FANTOM5 consortium (Forrest, A. R. R. et al. Nature 507, 462-470 (2014)). to select an appropriate background for DESeq (Anders, S. & Huber, W. (2010)). Genome Biol. 2010; 11(10):R106) to calculate the adjusted p-value and log fold change for genes in the sample. (C) For each TF we construct a local network neighborhood of influence weighting the downstream effect on a gene by its connected distance and the out-degree of its parent. (D) We maximise regulatory coverage by removing TFs which are redundant in their influence over other factors.

FIG. 2. Mogrify predictions for some of the known trans-differentiations that are published in the literature. TFs that Mogrify correctly identifies from the published list are highlighted. Samples are grouped using the FANTOM cell ontology (Forrest, A. R. R. et al, as above). For each publication the transcription factors that are in the initial maximum coverage set are shown in green and in the overall predicted Mogrify set in orange. For instance the transdifferentiation between fibroblast and myoblast (Lattanzi, L. et al. J. Clin. Invest. 101, 2119-28 (1998)) required only MYOD and this was identified by Mogrify.

FIG. 3. Empirical validation of novel conversions predicted by Mogrify: induction of keratinocytes from dermal fibroblasts. (A) The transcription factor network predicted by Mogrify to be involved in the dermal fibroblast to keratinocyte transdifferentiation. (B) An outline of the method used for the transdifferentiation assay. (C) qPCR analysis of the indicated markers in cells harvested at days 12-16 during transdifferentiation. All values are experimental replicates and are relative to gene expression in dermal fibroblasts (n=3, error bars depict s.e.m). (D) Brightfield and GFP images at day 24 showing the cobblestone morphology of transdifferentiated cells (upper panel) and GFP+ control cells (lower panel).

FIG. 4. Empirical validation of novel conversions predicted by Mogrify: induction of microvascular endothelial cells from keratinocytes. (A) A schematic representation of the transcription factor network predicted by Mogrify to be involved in the keratinocyte to microvascular endothelial cell transdifferentiation. (B) An outline of the method used for the transdifferentiation assay. (C) Flow cytometric analysis of CD31 expression at day 0, 14 and 18 of transdifferentiation. (D) qPCR analysis of the indicated expression markers in CD31+ cells harvested at day 18 of transdifferentiation. All values are experimental replicates and are relative to gene expression in keratinocytes (n=3, error bars depict s.e.m). (E) Immunofluorescence analysis of endothelial markers CD31 and VE-Cadherin at day 18 for vector free control cells (a) and transdifferentiating cells (b-f). Scale bar=50 μm.

FIG. 5. Comparison to published conversions. The added coverage value for each conversion as an additional transcription factor is added to the list showing that the coverage has always reached close to 100% within eight transcription factors.

FIG. 6. Benchmarking against existing cell conversion TF techniques. In order to show how the performance of Mogrify compares with other published methods for retrieving sets of TFs for cell conversions two statistics are reported. Firstly (top panel), the recovery rate of each of the techniques; A recovery rate of 100% means that the technique also found all of the sets of TFs that were used in the published conversion. As a result if that technique had been used to design the experiment then the known conversion set would have been discovered in the first iteration. For Mogrify this is the case for 6/10 of the published conversions, for CellNet and D'Allessio et al this is only true for 1/10 of the published conversions. Secondly (bottom panel) the average rank of the recovered TFs is plotted. Ignoring those TFs that were missed by each of the techniques this test shows how well each technique managed to prioritise the required TFs. With the exception of the conversion between Fibroblast and heart (cardiomyocytes) Mogrify performed the best in every case. In the case where none of the correct TFs were predicted no average rank is shown. This is the case for four conversions in CellNet and one for D'Alessio et al.

FIG. 7. The reprogramming landscape of human cell type. Samples are grouped using the cell ontology terms provided by: Forrest et al. as above. The expression profiles of the ontology terms that contain replicates are arranged in the X-Y plane using multidimensional scaling, resulting in cell types with similar expression profiles being close together. The height on the landscape is then calculated according to the normalized cumulative coverage of the top 8 TFs according to Mogrify, as such a conversion where the top ranked TF regulates all of the required genes the height would be 1 and the opposite would result in a height of 0.

FIG. 8. Empirical validation of novel conversions predicted by Mogrify: induction of endothelial cells from dermal fibroblasts. A: Immunofluorescence analysis of endothelial markers PeCAM and VE-cadehrin at day 18 of transdifferentiation. Scale bar, 25 μm. B: qPCR analysis showing expression levels of the endothelial associated genes VEGFR2 and VE-Cadherin at day 18 of transdifferentiation.

FIG. 9. Empirical validation of novel conversions predicted by Mogrify: induction of endothelial cells from hESC. A: Immunofluorescence analysis of endothelial markers PeCAM and VE-cadehrin at day 18 of transdifferentiation. Scale bar, 25 μm. B: qPCR analysis showing expression levels of the endothelial associated genes VEGFR2 and VE-Cadherin at day 18 of transdifferentiation.

FIG. 10. Induction of endothelial cells from hESC. A: Flow cytometry analysis of PeCAM expression at day 12 and 18 of transdifferentiation. FSC, forward scatter. B: Quantification of PeCAM-positive cells at day 18 of transdifferentiation. N=3

FIG. 11. Empirical validation of novel conversions predicted by Mogrify: induction of endothelial cells from hiPSC. A: Immunofluorescence analysis of endothelial markers PeCAM and VE-cadehrin at day 18 of transdifferentiation. Scale bar, 25 μm. B: qPCR analysis showing expression levels of the endothelial associated genes VEGFR2 and VE-Cadherin at day 18 of transdifferentiation.

FIG. 12. Induction of endothelial cells from hiPSC A: Flow cytometry analysis of PeCAM expression at day 12 and 18 of transdifferentiation. FSC, forward scatter. B: Quantification of PeCAM-positive cells at day 18 of transdifferentiation. N=3

FIG. 13. Empirical validation of novel conversions predicted by Mogrify: induction of astrocyte cells from fibroblasts. Immunofluorescence analysis of astrocyte marker GFAP at day 21 of transdifferentiation. Scale bar, 25 μm.

FIG. 14. Empirical validation of novel conversions predicted by Mogrify: induction of astrocyte cells from hESC. Immunofluorescence analysis of astrocyte marker GFAP at day 21 of transdifferentiation. Scale bar, 25 μm.

FIG. 15. Empirical validation of novel conversions predicted by Mogrify: induction of astrocyte cells from hiPSC. Immunofluorescence analysis of astrocyte marker GFAP at day 21 of transdifferentiation. Scale bar, 25 μm.

FIG. 16. Empirical validation of novel conversions predicted by Mogrify: induction of astrocyte cells from BM-MSC. Immunofluorescence analysis of astrocyte marker GFAP at day 21 of transdifferentiation. Scale bar, 25 μm.

FIG. 17. Empirical validation of novel conversions predicted by Mogrify: induction of keratinocyte cells from hESC. Immunofluorescence analysis of keratinocyte marker Pan-Keratin at day 21 of transdifferentiation. Scale bar, 25 μm.

FIG. 18. Empirical validation of novel conversions predicted by Mogrify: induction of keratinocyte cells from hiPSC. A: Immunofluorescence analysis of keratinocyte marker Keratin 14 (KRT14) at day 21 of transdifferentiation. Scale bar, 25 μm. B and C: qPCR analysis showing expression levels of the keratinocyte associated genes Keratin 14 (B) and Keratin 1 (C) at day 21 of transdifferentiation.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

For purposes of interpreting this specification, terms used in the singular will also include the plural and vice versa.

The invention provides a practical and efficient mechanism for systematically implementing cell conversions, facilitating the generalization of the reprogramming of human cells. The present invention combines gene expression data with regulatory network information to achieve what neither of which alone is sufficient to do—to reliably and accurately identify the transcription factors required to convert a source cell type to a cell that exhibits at least one characteristic of a target cell type. Further, in some embodiments the present invention provides a set of transcription factors for a conversion rather than a ranked list of all transcription factors.

Expression data for each gene in a sample may be determined by any known method, including those described herein. The data may be generated de novo or derived from an existing database.

Differential expression can be calculated using DESeq, edgeR, baySeq23, BBSeq24, NOISeq25 or QuasiSeq protocols or any other process known to those skilled in the art for determining the differential expression in one or more samples against a background or pair-wise comparison.

A tree-based background approach referred to in various methods of the invention is based on the principle to exclude cell types whose ontologies are very close whilst including others that are near in the tree to the background. This may be achieved by picking a point near to the top of the tree that would act as the breaking point. Samples in the same clade as the cell type being analyzed can be removed and those not in the same clade, but still below that point, can be included. The result of this is a set of samples that is broad enough to give reliable results but narrow enough that the statistical power is kept at a manageable level.

An alternative to the tree-based approach is Bayesian clustering, specifically the DGEclust approach described in Vavoulis et al. Genome Biology. 2015, 16:39.

In order to calculate a transcription factor's network-based sphere of influence any network or subnetwork that contains a source of network information relating to the interactions of a transcription factor that affect gene expression may be used. Typically, this is information relating to the interactions of a transcription factor with other biological molecules, such as DNA, RNA or protein. For example, any network information regarding the protein-DNA interactions between transcription factors with known binding sites in the promoter or regulatory region of a gene. An example of such a source of network information is the Motif Activity Response Analysis (MARA) (The FANTOM Consortium, Suzuki et al. 2009. Nat Genet 41: 553-5620). A further example of a source of network information is a database of protein-protein, protein-DNA, protein-RNA and/or biological pathway interactions. An example of such a source of network information is the STRING (Search Tool for the Retrieval of Interacting Genes/Proteins) database. Examples of databases and methods to calculate a transcription factor's network-based sphere of influence are described in the Examples. Preferably, any technique which identifies the transcriptional start site, such as cap analysis gene expression (CAGE) is used to generate the gene score when MARA derived networks are used to generate the network score.

The weighted sum of gene influence can be calculated over one or more networks to generate one or more influence lists. Preferably, at least two influence lists are generated, such as those described herein. The weighting that may be applied includes a weighting so that genes that are increasingly further from direct regulation have less of an impact on the network score (referred to herein as distance weighting), and a weighting to compensate for highly ubiquitous transcription factors and prevent them from receiving artificially high scores by regulating large number of barely-differentially expressed genes (referred to herein as edge weighting).

As used herein “Mogrify” refers to a method as described herein, for determining transcription factors required for the conversion of a source cell to a cell exhibiting at least one characteristic of a target cell. In any embodiment of the present invention, the Mogrify method can be implemented in a variety of computer processing systems, for example, a laptop computer, a netbook computer, a tablet computer, a smart phone, a desktop computer, a server computer. In one embodiment, computer systems comprise a processor and a data storage device, wherein said data storage device has stored thereon a series of computer readable media. In one aspect, the computer system can further comprise an algorithm for comparing the expression profiles between source and/or target cells. In one embodiment, computer readable media have stored thereon an expression profile or series of expression profiles from different cell types. In a further embodiment, the computer readable media have stored thereon details of the transcription factors involved in regulating a network of genes. It will be appreciated that the particular type of computer processing system will determine the appropriate hardware and architecture used.

Determining the gene and network scores may be by any method as described herein, including the Examples.

Ranking the transcription factors may be by any method described herein taking into account the scores, such as gene and network scores described herein, or influence lists as described herein. Preferably, a score or influence list based on differential expression analysis and/or a score or influence list based on the interactions of a transcription factor that directly and/or indirectly affect gene expression are used to rank the transcription factors.

To identify the set of TFs for a given conversion, the ranked lists from the source and target cell type are compared. If a TF from the target cell type list is already expressed in the source target then it may be removed from the list.

Removing transcriptionally redundant TFs from the ranked lists from each cell type may be by any method described herein including by comparing the lists of genes that each of the TFs directly regulates. For a given TF, if there is a higher-ranking TF that regulates over 98% of the genes that it would regulate, then it may be removed. The resulting predictions therefore include TFs that are diverse in their regulatory sphere of influence.

The process of reprogramming a cell alters the type of progeny a cell can produce and includes the distinct processes of forward programming and transdifferentiation. In some embodiments, forward programming of multipotent cells or pluripotent cells provides cells exhibiting at least one characteristic of a cell type having a more differentiated phenotype than the multipotent cell or pluripotent cell. In other embodiments, transdifferentiation of one somatic cell provides a cell exhibiting at least one characteristic of another somatic cell type.

The present invention provides compositions and methods for direct reprogramming or transdifferentiation of source cells to target cells, without the source cell becoming an induced pluripotent stem cell (iPS) intermediately prior to becoming a target cell. In comparison to iPS cell technology, transdifferentiation is highly efficient and poses a very low risk of teratoma formation for downstream applications. Moreover, transdifferentiation can be used in vivo for the direct conversion of one cell type into another, whereas iPS cell technology cannot.

A source cell may be any cell type described herein, including a somatic cell or a diseased cell. The somatic cell may be an adult cell or a cell derived from an adult which displays one or more detectable characteristics of an adult or non-embryonic cell. The diseased cell may be a cell displaying one or more detectable characteristics of a disease or condition, for example the diseased cell may be a cancer cell displaying one or more clinical or biochemical markers of a cancer. Examples of source cells include a hematopoietic cell, e.g. lymphocyte, myeloid cell; a buccal mucosa cell, an epidermal cell, a mesenchymal cell, a keratinocyte, a hepatocyte. Examples of source cells are shown in Table 4.

As used herein, the term “somatic cell” refers to any cell forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro. The somatic cells may be immortalized to provide an unlimited supply of cells, for example, by increasing the level of telomerase reverse transcriptase (TERT). For example, the level of TERT can be increased by increasing the transcription of TERT from the endogenous gene, or by introducing a transgene through any gene delivery method or system.

Unless otherwise indicated the methods for reprogramming somatic cells can be performed both in vivo and in vitro (where in vivo is practiced when somatic cells are present within a subject, and where in vitro is practiced using isolated somatic cells maintained in culture).

An embryonic cell, such as an embryonic stem cell, may be a cell derived from an embryonic cell line and not directly derived from an embryo or fetus. Alternatively, the embryonic cell may be derived from an embryo or fetus however the cell is obtained or isolated without destruction of, or any negative influence on the development of, the embryo or fetus.

Differentiated somatic cells, including cells from a fetal, newborn, juvenile or adult primate, including human, individual, are suitable source cells in the methods of the invention. Suitable somatic cells include, but are not limited to, bone marrow cells, epithelial cells, endothelial cells, fibroblast cells, hematopoietic cells, keratinocytes, hepatic cells, intestinal cells, mesenchymal cells, myeloid precursor cells and spleen cells. Alternatively, the somatic cells can be cells that can themselves proliferate and differentiate into other types of cells, including blood stem cells, muscle/bone stem cells, brain stem cells and liver stem cells. Suitable somatic cells are receptive, or can be made receptive using methods generally known in the scientific literature, to uptake of transcription factors including genetic material encoding the transcription factors. Uptake-enhancing methods can vary depending on the cell type and expression system. Exemplary conditions used to prepare receptive somatic cells having suitable transduction efficiency are well-known by those of ordinary skill in the art. The starting somatic cells can have a doubling time of about twenty-four hours.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein, refers to a population of cells that has been removed and separated from a mixed or heterogeneous population of cells. In some embodiments, an isolated population is a substantially pure population of cells as compared to the heterogeneous population from which the cells were isolated or enriched from.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified”, with regard to a population of target cells, refers to a population of cells that contain fewer than about 20%, more preferably fewer than about 15%, 10%, 8%, 7%, most preferably fewer than about 5%, 4%, 3%, 2%, 1%, or less than 1%, of cells that are not target cells or their progeny as defined by the terms herein.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting, for example, lung, breast, thyroid, lymphoid, gastrointestinal, and genito-urinary tract, as well as adenocarcinomas which include malignancies such as most colon cancers, renal-cell carcinoma, prostate cancer and/or testicular tumours, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term “carcinoma” also includes carcinosarcomas, e.g., which include malignant tumours composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

As used herein, reference to a “target cell” may be a reference to any one or more of the cells referred to herein as target cells or target cell types, such as those in the top row of Table 4.

A source cell is determined to be converted to a target cell, or become a target-like cell, by a method of the invention when it displays at least one characteristic of the target cell type. For example, a human fibroblast will be identified as converted to a keratinocyte-like cell, when a cell displays at least one characteristic of the target cell type. Typically, a cell will display 1, 2, 3, 4, 5, 6, 7, 8 or more characteristics of the target cell type. For example, where the target cell is a keratinocyte cell, a cell is identified or determined to be a keratinocyte-like cell when up-regulation of any one or more keratinocyte markers and/or change in cell morphology is detectable, preferably, the keratinocyte markers include keratin1, keratin14 and involucrin and the cell morphology is cobblestone appearance. In any aspect of the invention, the target cell characteristic may be determined by analysis of cell morphology, gene expression profiles, activity assay, protein expression profile, surface marker profile, or differentiation ability. Examples of characteristics or markers include those that are described herein and those known to the skilled person. Other examples of relevant markers include, for example for a conversion of keratinocytes to haemopoietic stem cells (HSC): CD45 (pan haematopoietic marker), CD19/20 (B-cell markers), CD14/15 (myeloid), CD34 (progenitor/SC markers), CD90 (SC) and alpha-integrin (keratinocyte marker not expressed by HSC); for human embryonic stem cells to haemopoietic stem cells: Runx1 (GFP), CD45 (pan haematopoietic marker), CD19/20 (B-cell markers), CD14/15 (myeloid), CD34 (progenitor/SC markers), CD90 (SC), Tra-1-160 (ESC marker not expressed in HSC); for rejuvenation of aged or adult HSC: a comparison between the transcriptional signatures of young and aged human HSC (e.g. using RNA-seq), and functional characterisation of “rejuvenated HSC” by transplanting rejuvenated cells into animals then assessed after 1, 3 and 6 months to determine the myeloid bias, wherein a disappearance of the myeloid bias indicates “rejuvenated” HSC. Examples of markers for many of the conversions described herein are shown in Table 1 below.

TABLE 1
Exemplary markers for target cells

| Target cell | Marker |
|---|---|
| Astrocytes | GFAP, S100B, ALDH1L1 |
| Chondrocytes | CD49. CD10, CD9, CD95, Integrin α10β1, 105 and |
| | Production of sulphated glycosaminoglycans (GAG) |
| Epithelial cells | cytokeratin 15 (CK15), cytokeratin 3 (CK3), |
| | involucrin and connexin 4. |
| Endothelial cells | VEGFR2, VE-Cadherin, Pe-CAM (CD31) |
| Hair follicles | CD200, PHLDA1, follistatin |
| Keratinocytes | Pan-keratin, Keratin 14, Keratin 1, involucrin |
| CD4+ T-cell | CD3, CD4 |
| CD8+ T-cell | CD3, CD8 |
| NK-cell | CD56, CD2 |
| HSCs | CD45 (pan haematopoietic marker), CD19/20 (B-cell |
| | markers), CD14/15 (myeloid), CD34 (progenitor/SC |
| | markers), CD90 (SC) |
| MSCs of adipose | CD13, CD29, CD90, CD105, CD10, CD45- and |
| | differentiate in vitro towards osteoblasts, adipocytes |
| | and chondrocytes |
| MSCs of bone | CD13, CD29, CD90, CD105, CD10, and |
| marrow | differentiate in vitro towards osteoblasts, adipocytes |
| | and chondrocytes |
| Oligodendrocytes | NG2 and PDGFRα QPCR for Olig2 and Nkx2.2 |
| precursor | |
| Skeletal muscle | MyoD, Myogenin and Desmin |
| cell | |
| Smooth muscle | Myocardin, Smooth Muscle Alpha Actin, Smooth |
| cell | muscle myosin heavy chain |
| Fetal | MEF2C, MYH6, ACTN1, CDH2 and GJA1 |
| cardiomyocytes | |

The transcription factors referred to herein are referred to by the HUGO Gene Nomenclature Committee (HGNC) Symbol. Exemplary nucleotide sequences for each transcription factor are shown in Tables 2 and 3 below. The nucleotide sequences are derived from the Ensembl database (Flicek et al. (2014). Nucleic Acids Research Volume 42, Issue D1. Pp. D749-D755) version 83. Also contemplated for use in the invention is any homolog, ortholog or paralog of a transcription factor referred to herein.

Table 2a and b below: Ensembl gene accession numbers (Nucleotide sequences; NT seq) and exemplary transcription factors (TF) that can be used in accordance with the methods described herein. Source cell types are shown in the far left column and target cell types in the top row. The transcription factors that may be used to convert the source cell type to a cell that exhibits at least one characteristic of the target cell type are shown.

| | Target cells | | | | | |
|---|---|---|---|---|---|---|
| | | Hair | | | | |
| | Chondrocytes | follicles | CD4+ T-cell | | NK-cell | |
| Source | TF and | TF and NT | TF and NT | CD8+ T-cell | TF and NT | HSCs |
| cells | NT seq | seq | seq | TF and NT seq | seq | TF and NT seq |
| Dermal Fibroblasts | BARX1 | ZIC1 | RORA | RORA | RORA | MYB |
| | ENSG00000131668 | ENSG00000152977 | ENSG00000069667 | ENSG00000069667 | ENSG00000069667 | ENSG00000118513 |
| | PITX1 | PRRX2 | LEF1 | FOS | SMAD7 | GATA1 |
| | ENSG00000069011 | ENSG00000167157 | ENSG00000138795 | ENSG00000170345 | ENSG00000101665 | ENSG00000102145 |
| | SMAD6 | RARB | JUN | SMAD7 | FOS | GFI1 |
| | ENSG00000137834 | ENSG00000077092 | ENSG00000177606 | ENSG00000101665 | ENSG00000170345 | ENSG00000162676 |
| | FOXC1 | VDR | FOS | JUN | JUN | GFI1B |
| | ENSG00000054598 | ENSG00000111424 | ENSG00000170345 | ENSG00000177606 | ENSG00000177606 | ENSG00000165702 |
| | SIX2 | FOXD1 | BACH2 | RUNX3 | NFATC2 | |
| | ENSG00000170577 | ENSG00000251493 | ENSG00000112182 | ENSG00000020633 | ENSG00000101096 | |
| | AHR | CREB3 | | | | |
| | ENSG00000106546 | ENSG00000107175 | | | | |

| | Target cells | | | | | |
|---|---|---|---|---|---|---|
| | | MSCs of | | | Smooth | |
| | | bone | | Skeletal | muscle | Fetal |
| | MSCs of | marrow | Oligodendrocytes | muscle cell | cell | cardio- |
| Source | adipose | TF and NT | precursor | TF and NT | TF and NT | myocytes |
| cells | TF and NT seq | seq | TF and NT seq | seq | seq | TF and NT seq |
| Dermal | NOTCH3 | SIX1 | NKX2-1 | MYOG | GATA6 | BMP10 |
| Fibroblasts | ENSG00000074181 | ENSG00000126778 | ENSG00000136352 | ENSG00000122180 | ENSG00000141448 | ENSG00000163217 |
| | HIC1 | ID1 | ANKRD1 | HIC1 | LIF | GATA6 |
| | ENSG00000177374 | ENSG00000125968 | ENSG00000148677 | ENSG00000177374 | ENSG00000128342 | ENSG00000141448 |
| | ID1 | HOXA7 | FOXA2 | MYOD1 | JUNB | TBX5 |
| | ENSG00000125968 | ENSG00000122592 | ENSG00000125798 | ENSG00000129152 | ENSG00000171223 | ENSG00000089225 |
| | ESRRA | FOXC2 | CDH1 | FOXD1 | CREB3 | FHL2 |
| | ENSG00000173153 | ENSG00000176692 | ENSG00000039068 | ENSG00000251493 | ENSG00000107175 | ENSG00000115641 |
| | IRF1 | HOXA9 | ZFP42 | PITX3 | MEIS1 | NKX2-5 |
| | ENSG00000125347 | ENSG00000078399 | ENSG00000179059 | ENSG00000107859 | ENSG00000143995 | ENSG00000183072 |
| | SIX5 | MAFB | IGF1 | SIX2 | PBX1 | HAND2 |
| | ENSG00000177045 | ENSG00000204103 | ENSG00000017427 | ENSG00000170577 | ENSG00000185630 | ENSG00000164107 |
| | SREBF1 | IRX5 | ICAM1 | HOXA7 | | GATA4 |
| | ENSG00000072310 | ENSG00000176842 | ENSG00000090339 | ENSG00000122592 | | ENSG00000136574 |
| | SNAI2 | | FOS | JUNB | | PPARGC1A |
| | ENSG00000019549 | | ENSG00000170345 | ENSG00000171223 | | ENSG00000109819) |

| | Target cells | | | | | |
|---|---|---|---|---|---|---|
| | Chondrocytes | Hair follicles | | | | |
| Source | TF and | TF and AA seq | CD4+ T-cell | CD8+ T-cell | NK-cell | HSCs |
| cells | NT seq | NT | TF and NT seq | TF and NT seq | TF and NT seq | TF and NT seq |
| Epidermal | BARX1 | RUNX1T1 | RORA | RORA | RORA | MYB |
| Keratinocytes | ENSG00000131668 | ENSG00000079102 | ENSG00000069667 | ENSG00000069667 | ENSG00000069667 | ENSG00000118513 |
| | PITX1 | ZIC1 | LEF1 | FOS | SMAD7 | GATA1 |
| | ENSG00000069011 | ENSG00000152977 | ENSG00000138795 | ENSG00000170345 | ENSG00000101665 | ENSG00000102145 |
| | SMAD6 | PRRX1 | JUN | SMAD7 | FOS | GFI1 |
| | ENSG00000137834 | ENSG00000116132 | ENSG00000177606 | ENSG00000101665 | ENSG00000170345 | ENSG00000162676 |
| | TGFB3 | MSX1 | FOS | JUN | JUN | GFI1B |
| | ENSG00000119699 | ENSG00000163132 | ENSG00000170345 | ENSG00000177606 | ENSG00000177606 | ENSG00000165702 |
| | FOXC1 | EBF1 | NR3C1 | RUNX3 | NFATC2 | |
| | ENSG00000054598 | ENSG00000164330 | ENSG00000113580 | ENSG00000020633 | ENSG00000101096 | |
| | SIX2 | FOXD1 | | | RUNX3 | |
| | ENSG00000170577 | ENSG00000251493 | | | ENSG00000020633 | |
| | | RUNX2 | | | | |
| | | ENSG00000124813 | | | | |
| h9 ESC line | BARX1 | TWIST1 | RORA | RORA | RORA | MYB |
| | ENSG00000131668 | ENSG00000122691 | ENSG00000069667 | ENSG00000069667 | ENSG00000069667 | ENSG00000118513 |
| | PITX1 | ZIC1 | LEF1 | FOS | SMAD7 | IL1B |
| | ENSG00000069011 | ENSG00000152977 | ENSG00000138795 | ENSG00000170345 | ENSG00000101665 | ENSG00000125538 |
| | SMAD6 | NR2F2 | JUN | SMAD7 | FOS | KLF1 |
| | ENSG00000137834 | ENSG00000185551 | ENSG00000177606 | ENSG00000101665 | ENSG00000170345 | ENSG00000105610 |
| | NFKB1 | PRRX1 | FOS | JUN | JUN | GATA1 |
| | ENSG00000109320 | ENSG00000116132 | ENSG00000170345 | ENSG00000177606 | ENSG00000177606 | ENSG00000102145 |
| | | NFKB1 | BACH2 | | NFATC2 | GFI1 |
| | | ENSG00000109320 | ENSG00000112182 | | ENSG00000101096 | ENSG00000162676 |
| | | AHR | | | | GFI1B |
| | | ENSG00000106546 | | | | ENSG00000165702 |
| | | | | | | NFE2 |
| | | | | | | ENSG00000123405 |
| Monocytes | | | | | | MYB |
| | | | | | | ENSG00000118513 |
| | | | | | | IL1B |
| | | | | | | ENSG00000125538 |
| | | | | | | GATA1 |
| | | | | | | ENSG00000102145 |
| | | | | | | GFI1 |
| | | | | | | ENSG00000162676 |
| | | | | | | GFI1B |
| | | | | | | ENSG00000165702 |

Target cells

| | MSCs of | MSCs of | Oligodendrocytes | Skeletal | Smooth |
|---|---|---|---|---|---|
| Source | adipose | bone marrow | precursor | muscle cell | muscle cell |
| cells | TF and NT seq | TF and NT seq | TF and NT seq | TF and NT seq | TF and NT seq |
| Epidermal | TWIST1 | SIX1 | NKX2-1 | MYOG | IRF1 |
| Keratinocytes | ENSG00000122691 | ENSG00000126778 | ENSG00000136352 | ENSG00000122180 | ENSG00000125347 |
| | HIC1 | TWIST1 | ANKRD1 | MYOD1 | GATA6 |
| | ENSG00000177374 | ENSG00000122691 | ENSG00000148677 | ENSG00000129152 | ENSG00000141448 |
| | ID1 | ID1 | ZFP42 | IRF1 | LIF |
| | ENSG00000125968 | ENSG00000125968 | ENSG00000179059 | ENSG00000125347 | ENSG00000128342 |
| | MSX1 | HMOX1 | FOS | PITX3 | MEIS1 |
| | ENSG00000163132 | ENSG00000100292 | ENSG00000170345 | ENSG00000107859 | ENSG00000143995 |
| | IRF1 | FOXC2 | IGF1 | HOXA7 | |
| | ENSG00000125347 | ENSG00000176692 | ENSG00000017427 | ENSG00000122592 | |
| | HOXB7 | HOXA7 | ICAM1 | FOXD1 | |
| | ENSG00000260027 | ENSG00000122592 | ENSG00000090339 | ENSG00000251493 | |
| | SNAI2 | | FOXA2 | SOX8 | |
| | ENSG00000019549 | | ENSG00000125798 | ENSG00000005513 | |
| | E2F1 | | CDH1 | | |
| | ENSG00000101412 | | ENSG00000039068 | | |
| h9 ESC line | TWIST1 | IRF1 | NKX2-1 | MYOG | IRF1 |
| | ENSG00000122691 | ENSG00000125347 | ENSG00000136352 | ENSG00000122180 | ENSG00000125347 |
| | SNAI2 | RUNX1 | ANKRD1 | IRF1 | NFKB1 |
| | ENSG00000019549 | ENSG00000159216 | ENSG00000148677 | ENSG00000125347 | ENSG00000109320 |
| | IRF1 | CEBPB | FOXA2 | MYOD1 | JUNB |
| | ENSG00000125347 | ENSG00000172216 | ENSG00000125798 | ENSG00000129152 | ENSG00000171223 |
| | MXD4 | AHR | LMO3 | FOXD1 | FOSL2 |
| | ENSG00000123933 | ENSG00000106546 | ENSG00000048540 | ENSG00000251493 | ENSG00000075426 |
| | NFKB1 | FOXC2 | FOS | NFKB1 | GATA6 |
| | ENSG00000109320 | ENSG00000176692 | ENSG00000170345 | ENSG00000109320 | ENSG00000141448 |
| | MSX1 | HOXA9 | IGF1 | JUNB | MEIS1 |
| | ENSG00000163132 | ENSG00000078399 | ENSG00000017427 | ENSG00000171223 | ENSG00000143995 |
| | HOXB7 | | ICAM1 | HOXA7 | |
| | ENSG00000260027 | | ENSG00000090339 | ENSG00000122592 | |
| | ESRRA | | CDH1 | | |
| | ENSG00000173153 | | ENSG00000039068 | | |
| Monocytes | | | | | |

| | Target cells |
|---|---|
| Source | Fetal cardiomyocytes |
| cells | TF and NT seq |
| Cardiac fibroblast | BMP10 ENSG00000163217 |
| | GATA6 ENSG00000141448 |
| | TBX5 ENSG00000089225 |
| | ANKRD1 ENSG00000148677 |
| | HAND1 ENSG00000113196 |
| | PPARGC1A ENSG00000109819 |
| | NKX2-5 ENSG00000183072 |
| | GATA4 ENSG00000136574 |

The term a “variant” in referring to a polypeptide that is at least 70%, 80%, 85%, 90%, 95%, 98%, or 99% identical to the full length polypeptide. The present invention contemplates the use of variants of the transcription factors described herein, including the sequences listed in Table 2a and b. The variant could be a fragment of full length polypeptide or a naturally occurring splice variant. The variant could be a polypeptide at least 70%, 80%, 85%, 90%, 95%, 98%, or 99% identical to a fragment of the polypeptide, wherein the fragment is at least 50%, 60%, 70%, 80%, 85%, 90%, 95%, 98%, or 99% as long as the full length wild type polypeptide or a domain thereof has a functional activity of interest such as the ability to promote conversion of a source cell type to a target cell type. In some embodiments the domain is at least 100, 200, 300, or 400 amino acids in length, beginning at any amino acid position in the sequence and extending toward the C-terminus. Variations known in the art to eliminate or substantially reduce the activity of the protein are preferably avoided. In some embodiments, the variant lacks an N- and/or C-terminal portion of the full length polypeptide, e.g., up to 10, 20, or 50 amino acids from either terminus is lacking. In some embodiments the polypeptide has the sequence of a mature (full length) polypeptide, by which is meant a polypeptide that has had one or more portions such as a signal peptide removed during normal intracellular proteolytic processing (e.g., during co-translational or post-translational processing). In some embodiments wherein the protein is produced other than by purifying it from cells that naturally express it, the protein is a chimeric polypeptide, by which is meant that it contains portions from two or more different species. In some embodiments wherein a protein is produced other than by purifying it from cells that naturally express it, the protein is a derivative, by which is meant that the protein comprises additional sequences not related to the protein so long as those sequences do not substantially reduce the biological activity of the protein. One of skill in the art will be aware of, or will readily be able to ascertain, whether a particular polypeptide variant, fragment, or derivative is functional using assays known in the art. For example, the ability of a variant of a transcription factor to convert a source cell to a target cell type can be assessed using the assays as disclose herein in the Examples. Other convenient assays include measuring the ability to activate transcription of a reporter construct containing a transcription factor binding site operably linked to a nucleic acid sequence encoding a detectable marker such as luciferase. In certain embodiments of the invention a functional variant or fragment has at least 50%, 60%, 70%, 80%, 90%, 95% or more of the activity of the full length wild type polypeptide.

The term “increasing the amount of” with respect to increasing an amount of a transcription factor, refers to increasing the quantity of the transcription factor in a cell of interest (e.g., a source cell such as a fibroblast or keratinocyte cell). In some embodiments, the amount of transcription factor is “increased” in a cell of interest (e.g., a cell into which an expression cassette directing expression of a polynucleotide encoding one or more transcription factors has been introduced) when the quantity of transcription factor is at least 10%, 20%, 30%, 40%, 50%, 60%, 70%, 80%, 90%, or more relative to a control (e.g., a fibroblast or keratinocyte cell into which none of said expression cassettes have been introduced). However, any method of increasing an amount of a transcription factor is contemplated including any method that increases the amount, rate or efficiency of transcription, translation, stability or activity of a transcription factor (or the pre-mRNA or mRNA encoding it). In addition, down-regulation or interference of a negative regulator of transcription expression, increasing efficiency of existing transcription (e.g. SINEUP) are also considered.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

The term “exogenous,” when used in relation to a protein, gene, nucleic acid, or polynucleotide in a cell or organism refers to a protein, gene, nucleic acid, or polynucleotide that has been introduced into the cell or organism by artificial or natural means; or in relation to a cell, refers to a cell that was isolated and subsequently introduced to other cells or to an organism by artificial or natural means. An exogenous nucleic acid may be from a different organism or cell, or it may be one or more additional copies of a nucleic acid that occurs naturally within the organism or cell. An exogenous cell may be from a different organism, or it may be from the same organism. By way of a non-limiting example, an exogenous nucleic acid is one that is in a chromosomal location different from that of natural cells, or is otherwise flanked by a different nucleic acid sequence than that found in nature. An exogenous nucleic acid may also be extra-chromosomal, such as an episomal vector.

Screening one or more candidate agents for the ability to increase the amount of the one or more transcription factors required for conversion of a source cell type to a target cell type may include the steps of contacting a system that allows the product or expression of a transcription factor with the candidate agent and determining whether the amount of the transcription factor has increased. The system may be in vivo, for example a tissue or cell in an organism, or in vitro, a cell isolated from an organism or an in vitro transcription assay, or ex vivo in a cell or tissue. The amount of transcription factor may be measured directly or indirectly, and either by determining the amount of protein or RNA (e.g. mRNA or pre-mRNA). The candidate agent function to increase the amount of a transcription factor by increasing any step in the transcription of the gene encoding the transcription factor or increase the translation of corresponding mRNA. Alternatively, the candidate agent may decrease the inhibitory activity of a repressor of transcription of the gene encoding the transcription factor or the activity of a molecule that causes the degradation of the mRNA encoding the transcription factor or the protein of the transcription factor itself.

Suitable detection means include the use of labels such as radionucleotides, enzymes, coenzymes, fluorescers, chemiluminescers, chromogens, enzyme substrates or co-factors, enzyme inhibitors, prosthetic group complexes, free radicals, particles, dyes, and the like. Such labelled reagents may be used in a variety of well-known assays, such as radioimmunoassays, enzyme immunoassays, e.g., ELISA, fluorescent immunoassays, and the like. See, for example, U.S. Pat. Nos. 3,766,162; 3,791,932; 3,817,837; and 4,233,402.

The methods of the invention include high-throughput screening applications. For example, a high-throughput screening assay may be used which comprises any of the assays according to the invention wherein aliquots of a system that allows the product or expression of a transcription factor are exposed to a plurality of candidate agents within different wells of a multi-well plate. Further, a high-throughput screening assay according to the disclosure involves aliquots of a system that allows the product or expression of a transcription factor which are exposed to a plurality of candidate agents in a miniaturized assay system of any kind.

The method of the disclosure may be “miniaturized” in an assay system through any acceptable method of miniaturization, including but not limited to multi-well plates, such as 24, 48, 96 or 384-wells per plate, microchips or slides. The assay may be reduced in size to be conducted on a micro-chip support, advantageously involving smaller amounts of reagent and other materials. Any miniaturization of the process which is conducive to high-throughput screening is within the scope of the invention.

In any method of the invention the target cells can be transferred into the same mammal from which the source cells were obtained. In other words, the source cells used in a method of the invention can be an autologous cell, i.e., can be obtained from the same individual in which the target cells are to be administered. Alternatively, the target cell can be allogenically transferred into another individual. Preferably, the cell is autologous to the subject in a method of treating or preventing a medical condition in the individual.

The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art. Exemplary cell culture medium for use in methods of the invention are shown in Table 9.

TABLE 3
Accession numbers identifying nucleotide sequences and amino acid
sequences of transcription factors referred to herein.

| Associated Gene | | |
|---|---|---|
| Name | Ensembl Gene ID | Uniprot ID |
| NOTCH3 | ENSG00000074181 | Q9UM47 |
| PRRX2 | ENSG00000167157 | Q99811 |
| HOXB7 | ENSG00000260027 | P09629 |
| BACH2 | ENSG00000112182 | Q9BYV9 |
| IL1B | ENSG00000125538 | P01584 |
| IRF1 | ENSG00000125347 | P10914 |
| HOXA9 | ENSG00000078399 | P31269 |
| BARX1 | ENSG00000131668 | Q9HBU1 |
| CEBPB | ENSG00000172216 | P17676 |
| RUNX1T1 | ENSG00000079102 | Q06455 |
| ZFP42 | ENSG00000179059 | Q96MM3 |
| LIF | ENSG00000128342 | P15018 |
| IGF1 | ENSG00000017427 | P05019 |
| PITX1 | ENSG00000069011 | P78337 |
| SMAD7 | ENSG00000101665 | O15105 |
| RORA | ENSG00000069667 | P35398 |
| MYB | ENSG00000118513 | P10242 |
| NFE2 | ENSG00000123405 | Q16621 |
| SIX1 | ENSG00000126778 | Q15475 |
| HIC1 | ENSG00000177374 | Q14526 |
| FOXC2 | ENSG00000176692 | Q99958 |
| SIX5 | ENSG00000177045 | Q8N196 |
| JUN | ENSG00000177606 | P05412 |
| HMOX1 | ENSG00000100292 | P09601 |
| LMO3 | ENSG00000048540 | Q8TAP4 |
| NR3C1 | ENSG00000113580 | P04150 |
| PITX3 | ENSG00000107859 | O75364 |
| VDR | ENSG00000111424 | P11473 |
| NR2F2 | ENSG00000185551 | P24468 |
| KLF1 | ENSG00000105610 | Q13351 |
| FOXC1 | ENSG00000054598 | Q12948 |
| HOXA7 | ENSG00000122592 | P31268 |
| AHR | ENSG00000106546 | P35869 |
| RARB | ENSG00000077092 | P10826 |
| GATA6 | ENSG00000141448 | Q92908 |
| TGFB3 | ENSG00000119699 | P10600 |
| FOSL2 | ENSG00000075426 | P15408 |
| MYOG | ENSG00000122180 | P15173 |
| MYOD1 | ENSG00000129152 | P15172 |
| MAFB | ENSG00000204103 | Q9Y5Q3 |
| IRX5 | ENSG00000176842 | P78411 |
| GFI1B | ENSG00000165702 | Q5VTD9 |
| LEF1 | ENSG00000138795 | Q9UJU2 |
| E2F1 | ENSG00000101412 | Q01094 |
| SIX2 | ENSG00000170577 | Q9NPC8 |
| ICAM1 | ENSG00000090339 | P05362 |
| RUNX2 | ENSG00000124813 | Q13950 |
| FOS | ENSG00000170345 | P01100 |
| PRRX1 | ENSG00000116132 | P54821 |
| ESRRA | ENSG00000173153 | P11474 |
| ID1 | ENSG00000125968 | P41134 |
| NFATC2 | ENSG00000101096 | Q13469 |
| SMAD6 | ENSG00000137834 | O43541 |
| TWIST1 | ENSG00000122691 | Q15672 |
| MEIS1 | ENSG00000143995 | O00470 |
| MSX1 | ENSG00000163132 | P28360 |
| CDH1 | ENSG00000039068 | P12830 |
| JUNB | ENSG00000171223 | P17275 |
| SNAI2 | ENSG00000019549 | O43623 |
| RUNX3 | ENSG00000020633 | Q13761 |
| CREB3 | ENSG00000107175 | O43889 |
| GFI1 | ENSG00000162676 | Q99684 |
| SOX8 | ENSG00000005513 | P57073 |
| EBF1 | ENSG00000164330 | Q9UH73 |
| PBX1 | ENSG00000185630 | P40424 |
| RUNX1 | ENSG00000159216 | Q01196 |
| ANKRD1 | ENSG00000148677 | Q15327 |
| FOXD1 | ENSG00000251493 | Q16676 |
| SREBF1 | ENSG00000072310 | P36956 |
| NKX2-1 | ENSG00000136352 | P43699 |
| NFKB1 | ENSG00000109320 | P19838 |
| ZIC1 | ENSG00000152977 | Q15915 |
| MXD4 | ENSG00000123933 | Q14582 |
| FOXA2 | ENSG00000125798 | Q9Y261 |
| GATA1 | ENSG00000102145 | P15976 |
| TFAP2A | ENSG00000137203 | P05549 |
| REL | ENSG00000162924 | Q04864 |
| CDH1 | ENSG00000039068 | P12830 |
| FOXQ1 | ENSG00000164379 | Q9C009 |
| FOSL2 | ENSG00000075426 | P15408 |
| MYC | ENSG00000136997 | P01106 |
| MYBL2 | ENSG00000101057 | P10244 |
| HMGB2 | ENSG00000164104 | P26583 |
| PAX6 | ENSG00000007372 | P26367 |
| SOX17 | ENSG00000164736 | Q9H6I2 |
| SMAD1 | ENSG00000170365 | Q15797 |
| TAL1 | ENSG00000162367 | P17542 |
| IRF1 | ENSG00000125347 | P10914 |
| TCF7L1 | ENSG00000152284 | Q9HCS4 |
| SOX2 | ENSG00000181449 | P48431 |
| SOX9 | ENSG00000125398 | P48436 |
| ARNT2 | ENSG00000172379 | Q9HBZ2 |
| E2F5 | ENSG00000133740 | Q15329 |
| FOSL1 | ENSG00000175592 | P15407 |
| SOX5 | ENSG00000134532 | P35711 |
| TP63 | ENSG00000073282 | Q9H3D4 |
| NFKBIA | ENSG00000100906 | P25963 |
| POU3F2 | ENSG00000184486 | P20265 |
| DBP | ENSG00000105516 | Q10586 |
| HES1 | ENSG00000114315 | Q14469 |
| NOTCH1 | ENSG00000148400 | P46531 |
| HR | ENSG00000168453 | O43593 |
| OTX1 | ENSG00000115507 | P32242 |

A nucleic acid or vector comprising a nucleic acid as described herein may include one or more of the sequences referred to above in Table 3 or a sequence encoding any one or more of the amino acid sequences listed in Table 3.

The term “expression” refers to the cellular processes involved in producing RNA and proteins and as appropriate, secreting proteins, including where applicable, but not limited to, for example, transcription, translation, folding, modification and processing.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated”.

The term “vector” refers to a carrier DNA molecule into which a DNA sequence can be inserted for introduction into a host or source cell. Preferred vectors are those capable of autonomous replication and/or expression of nucleic acids to which they are linked. Vectors capable of directing the expression of genes to which they are operatively linked are referred to herein as “expression vectors”. Thus, an “expression vector” is a specialized vector that contains the necessary regulatory regions needed for expression of a gene of interest in a host cell. In some embodiments the gene of interest is operably linked to another sequence in the vector. Vectors can be viral vectors or non-viral vectors. Should viral vectors be used, it is preferred the viral vectors are replication defective, which can be achieved for example by removing all viral nucleic acids that encode for replication. A replication defective viral vector will still retain its infective properties and enters the cells in a similar manner as a replicating adenoviral vector, however once admitted to the cell a replication defective viral vector does not reproduce or multiply. Vectors also encompass liposomes and nanoparticles and other means to deliver DNA molecule to a cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. The term “operatively linked” includes having an appropriate start signal (e.g. ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

The term “viral vectors” refers to the use of viruses, or virus-associated vectors as carriers of a nucleic acid construct into a cell. Constructs may be integrated and packaged into non-replicating, defective viral genomes like Adenovirus, Adeno-associated virus (AAV), or Herpes simplex virus (HSV) or others, including reteroviral and lentiviral vectors, for infection or transduction into cells. The vector may or may not be incorporated into the cell's genome. The constructs may include viral sequences for transfection, if desired. Alternatively, the construct may be incorporated into vectors capable of episomal replication, e.g EPV and EBV vectors.

As used herein, the term “adenovirus” refers to a virus of the family Adenovirida. Adenoviruses are medium-sized (90-100 nm), nonenveloped (naked) icosahedral viruses composed of a nucleocapsid and a double-stranded linear DNA genome.

As used herein, the term “non-integrating viral vector” refers to a viral vector that does not integrate into the host genome; the expression of the gene delivered by the viral vector is temporary. Since there is little to no integration into the host genome, non-integrating viral vectors have the advantage of not producing DNA mutations by inserting at a random point in the genome. For example, a non-integrating viral vector remains extra-chromosomal and does not insert its genes into the host genome, potentially disrupting the expression of endogenous genes. Non-integrating viral vectors can include, but are not limited to, the following: adenovirus, alphavirus, picornavirus, and vaccinia virus. These viral vectors are “non-integrating” viral vectors as the term is used herein, despite the possibility that any of them may, in some rare circumstances, integrate viral nucleic acid into a host cell's genome. What is critical is that the viral vectors used in the methods described herein do not, as a rule or as a primary part of their life cycle under the conditions employed, integrate their nucleic acid into a host cell's genome.

The vectors described herein can be constructed and engineered using methods generally known in the scientific literature to increase their safety for use in therapy, to include selection and enrichment markers, if desired, and to optimize expression of nucleotide sequences contained thereon. The vectors should include structural components that permit the vector to self-replicate in the source cell type. For example, the known Epstein Barr oriP/Nuclear Antigen-1 (EBNA-I) combination (see, e.g., Lindner, S. E. and B. Sugden, The plasmid replicon of Epstein-Barr virus: mechanistic insights into efficient, licensed, extrachromosomal replication in human cells, Plasmid 58:1 (2007), incorporated by reference as if set forth herein in its entirety) is sufficient to support vector self-replication and other combinations known to function in mammalian, particularly primate, cells can also be employed. Standard techniques for the construction of expression vectors suitable for use in the present invention are well-known to one of ordinary skill in the art and can be found in publications such as Sambrook J, et al., “Molecular cloning: a laboratory manual,” (3rd ed. Cold Spring harbor Press, Cold Spring Harbor, N.Y. 2001), incorporated herein by reference as if set forth in its entirety.

In the methods of the invention, genetic material encoding the relevant transcription factors required for a conversion is delivered into the source cells via one or more reprogramming vectors. Each transcription factor can be introduced into the source cells as a polynucleotide transgene that encodes the transcription factor operably linked to a heterologous promoter that can drive expression of the polynucleotide in the source cell.

Suitable reprogramming vectors are any described herein, including episomal vectors, such as plasmids, that do not encode all or part of a viral genome sufficient to give rise to an infectious or replication-competent virus, although the vectors can contain structural elements obtained from one or more virus. One or a plurality of reprogramming vectors can be introduced into a single source cell. One or more transgenes can be provided on a single reprogramming vector. One strong, constitutive transcriptional promoter can provide transcriptional control for a plurality of transgenes, which can be provided as an expression cassette. Separate expression cassettes on a vector can be under the transcriptional control of separate strong, constitutive promoters, which can be copies of the same promoter or can be distinct promoters. Various heterologous promoters are known in the art and can be used depending on factors such as the desired expression level of the transcription factor. It can be advantageous, as exemplified below, to control transcription of separate expression cassettes using distinct promoters having distinct strengths in the source cells. Another consideration in selection of the transcriptional promoters is the rate at which the promoter(s) is silenced. The skilled artisan will appreciate that it can be advantageous to reduce expression of one or more transgenes or transgene expression cassettes after the product of the gene(s) has completed or substantially completed its role in the reprogramming method. Exemplary promoters are the human EF1α elongation factor promoter, CMV cytomegalovirus immediate early promoter and CAG chicken albumin promoter, and corresponding homologous promoters from other species. In human somatic cells, both EF1α and CMV are strong promoters, but the CMV promoter is silenced more efficiently than the EF1α promoter such that expression of transgenes under control of the former is turned off sooner than that of transgenes under control of the latter. The transcription factors can be expressed in the source cells in a relative ratio that can be varied to modulate reprogramming efficiency. Preferably, where a plurality of transgenes is encoded on a single transcript, an internal ribosome entry site is provided upstream of transgene(s) distal from the transcriptional promoter. Although the relative ratio of factors can vary depending upon the factors delivered, one of ordinary skill in possession of this disclosure can determine an optimal ratio of factors.

The skilled artisan will appreciate that the advantageous efficiency of introducing all factors via a single vector rather than via a plurality of vectors, but that as total vector size increases, it becomes increasingly difficult to introduce the vector. The skilled artisan will also appreciate that position of a transcription factor on a vector can affect its temporal expression, and the resulting reprogramming efficiency. As such, Applicants employed various combinations of factors on combinations of vectors. Several such combinations are here shown to support reprogramming.

After introduction of the reprogramming vector(s) and while the source cells are being reprogrammed, the vectors can persist in target cells while the introduced transgenes are transcribed and translated. Transgene expression can be advantageously downregulated or turned off in cells that have been reprogrammed to a target cell type. The reprogramming vector(s) can remain extra-chromosomal. At extremely low efficiency, the vector(s) can integrate into the cells' genome. The examples that follow are intended to illustrate but in no way limit the present invention.

Suitable methods for nucleic acid delivery for transformation of a cell, a tissue or an organism for use with the current invention are believed to include virtually any method by which a nucleic acid (e.g., DNA) can be introduced into a cell, a tissue or an organism, as described herein or as would be known to one of ordinary skill in the art (e.g., Stadtfeld and Hochedlinger, Nature Methods 6(5):329-330 (2009); Yusa et al., Nat. Methods 6:363-369 (2009); Woltjen, et al., Nature 458, 766-770 (9 Apr. 2009)). Such methods include, but are not limited to, direct delivery of DNA such as by ex vivo transfection (Wilson et al., Science, 244:1344-1346, 1989, Nabel and Baltimore, Nature 326:711-713, 1987), optionally with a lipid-based transfection reagent such as Fugene6 (Roche) or Lipofectamine (Invitrogen), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, J. Cell Biol., 101:1094-1099, 1985; U.S. Pat. No. 5,789,215, incorporated herein by reference); by electroporation (U.S. Pat. No. 5,384,253, incorporated herein by reference; Tur-Kaspa et al., Mol. Cell Biol., 6:716-718, 1986; Potter et al., Proc. Nat'l Acad. Sci. USA, 81:7161-7165, 1984); by calcium phosphate precipitation (Graham and Van Der Eb, Virology, 52:456-467, 1973; Chen and Okayama, Mol. Cell Biol., 7(8):2745-2752, 1987; Rippe et al., Mol. Cell Biol., 10:689-695, 1990); by using DEAE-dextran followed by polyethylene glycol (Gopal, Mol. Cell Biol., 5:1188-1190, 1985); by direct sonic loading (Fechheimer et al., Proc. Nat'l Acad. Sci. USA, 84:8463-8467, 1987); by liposome mediated transfection (Nicolau and Sene, Biochim. Biophys. Acta, 721:185-190, 1982; Fraley et al., Proc. Nat'l Acad. Sci. USA, 76:3348-3352, 1979; Nicolau et al., Methods Enzymol., 149:157-176, 1987; Wong et al., Gene, 10:87-94, 1980; Kaneda et al., Science, 243:375-378, 1989; Kato et al., J Biol. Chem., 266:3361-3364, 1991) and receptor-mediated transfection (Wu and Wu, Biochemistry, 27:887-892, 1988; Wu and Wu, J. Biol. Chem., 262:4429-4432, 1987); and any combination of such methods, each of which is incorporated herein by reference.

A number of polypeptides capable of mediating introduction of associated molecules into a cell have been described previously and can be adapted to the present invention. See, e.g., Langel (2002) Cell Penetrating Peptides: Processes and Applications, CRC Press, Pharmacology and Toxicology Series. Examples of polypeptide sequences that enhance transport across membranes include, but are not limited to, the Drosophila homeoprotein antennapedia transcription protein (AntHD) (Joliot et al., New Biol. 3: 1121-34, 1991; Joliot et al., Proc. Natl. Acad. Sci. USA, 88: 1864-8, 1991; Le Roux et al., Proc. Natl. Acad. Sci. USA, 90: 9120-4, 1993), the herpes simplex virus structural protein VP22 (Elliott and O'Hare, Cell 88: 223-33, 1997); the HIV-1 transcriptional activator TAT protein (Green and Loewenstein, Cell 55: 1179-1188, 1988; Frankel and Pabo, Cell 55: 1 289-1193, 1988); Kaposi FGF signal sequence (kFGF); protein transduction domain-4 (PTD4); Penetratin, M918, Transportan-10; a nuclear localization sequence, a PEP-I peptide; an amphipathic peptide (e.g., an MPG peptide); delivery enhancing transporters such as described in U.S. Pat. No. 6,730,293 (including but not limited to an peptide sequence comprising at least 5-25 or more contiguous arginines or 5-25 or more arginines in a contiguous set of 30, 40, or 50 amino acids; including but not limited to an peptide having sufficient, e.g., at least 5, guanidino or amidino moieties); and commercially available Penetratin™ 1 peptide, and the Diatos Peptide Vectors (“DPVs”) of the Vectocell® platform available from Daitos S.A. of Paris, France. See also, WO/2005/084158 and WO/2007/123667 and additional transporters described therein. Not only can these proteins pass through the plasma membrane but the attachment of other proteins, such as the transcription factors described herein, is sufficient to stimulate the cellular uptake of these complexes.

TABLE 4a
Exemplary conversions of the present invention. Source cell types are
shown in the far left column and target cell types in the top row. The transcription factors
required to covert the source cell type to a cell that exhibits at least one characteristic of
the target cell type are shown.

| | Target cells | | | | | | |
|---|---|---|---|---|---|---|---|
| | | Hair | CD4+ T- | CD8+ T- | | | MSCs of |
| Source cells | Chondrocytes | follicles | cell | cell | NK-cell | HSCs | adipose |
| Dermal | BARX1 | ZIC1 | RORA | RORA | RORA | MYB | NOTCH3 |
| Fibroblasts | PITX1 | PRRX2 | LEF1 | FOS | SMAD7 | GATA1 | HIC1 |
| | SMAD6 | RARB | JUN | SMAD7 | FOS | GFI1 | ID1 |
| | FOXC1 | VDR | FOS | JUN | JUN | GFI1B | ESRRA |
| | SIX2 | FOXD1 | BACH2 | RUNX3 | NFATC2 | | IR1 |
| | AHR | CREB3 | | | | | SIX5 |
| | | | | | | | SREBF1 |
| | | | | | | | SNAI2 |
| Epidermal | BARX1 | RUNX1T1 | RORA | RORA | RORA | MYB | TWIST1 |
| Keratinocytes | PITX1 | ZIC1 | LEF1 | FOS | SMAD7 | GATA1 | HIC1 |
| | SMAD6 | PRRX1 | JUN | SMAD7 | FOS | GFI1 | ID1 |
| | TGFB3 | MSX1 | FOS | JUN | JUN | GFI1B | MSX1 |
| | FOXC1 | EBF1 | NR3C1 | RUNX3 | NFATC2 | | IRF1 |
| | SIX2 | FOXD1 | | | RUNX3 | | HOXB7 |
| | | RUNX2 | | | | | SNAI2 |
| | | | | | | | E2F1 |
| h9 ESC line | BARX1 | TWIST1 | RORA | RORA | RORA | MYB | TWIST1 |
| | PITX1 | ZIC1 | LEF1 | FOS | SMAD7 | IL1B | SNAI2 |
| | SMAD6 | NR2F2 | JUN | SMAD7 | FOS | KLF1 | IRF1 |
| | NFKB1 | PRRX1 | FOS | JUN | JUN | GATA1 | MXD4 |
| | | NFKB1 | BACH2 | | NFATC2 | GFI1 | NFKB1 |
| | | AHR | | | | GFI1B | MSX1 |
| | | | | | | NFE2 | HOXB7 |
| | | | | | | | ESRRA |
| Monocytes | | | | | | MYB | |
| | | | | | | IL1B | |
| | | | | | | GATA1 | |
| | | | | | | GFI1 | |
| | | | | | | GFI1B | |
| Cardiac | | | | | | | |
| fibroblasts | | | | | | | |

| | Target cells | | | | |
|---|---|---|---|---|---|
| | MSCs of | | | | Fetal |
| | bone | Oligodendrocytes | Skeletal | Smooth | cardio- |
| Source cells | marrow | precursor | muscle cell | muscle cell | myocytes |
| Dermal | SIX1 | NKX2-1 | MYOG | GATA6 | BMP10 |
| Fibroblasts | ID1 | ANKRD1 | HIC1 | LIF | GATA6 |
| | HOXA7 | FOXA2 | MYOD1 | JUNB | TBX5 |
| | FOXC2 | CDH1 | FOXD1 | CREB3 | FHL2 |
| | HOXA9 | ZIP42 | PITX3 | MEIS1 | NKX2-5 |
| | MAFB | IGF1 | SIX2 | PBX1 | HAND2 |
| | IRX5 | ICAM1 | HOXA7 | | GATA4 |
| | | FOS | JUNB | | PPARGC1A |
| Epidermal | SIX1 | NKX2-1 | MYOG | IRF1 | |
| Keratinocytes | TWSIT1 | ANKRD1 | MYOD1 | GATA6 | |
| | ID1 | ZFP42 | RF1 | LIF | |
| | HMOX1 | FOS | PITX3 | MEIS1 | |
| | FOXC2 | IGF1 | HOXA7 | | |
| | HOXA7 | ICAM1 | FOXD1 | | |
| | | FOXA2 | SOX8 | | |
| | | CDH1 | | | |
| h9 ESC line | IRF1 | NKX2-1 | MYOG | IRF1 | |
| | RUNX1 | ANKRD1 | IRF1 | NFKB1 | |
| | CEBPB | FOXA2 | MYOD1 | JUNB | |
| | AHR | LMO3 | FOXD1 | FOSL2 | |
| | FOXC2 | FOS | NFKB1 | GATA6 | |
| | HOXA9 | IGF1 | JUNB | MEIS1 | |
| | | ICAM1 | HOXA7 | | |
| | | CDH1 | | | |
| Monocytes | | | | | |
| Cardiac | | | | | BMP10 |
| fibroblasts | | | | | GATA6 |
| | | | | | TBX5 |
| | | | | | ANKRD1 |
| | | | | | HAND1 |
| | | | | | PPARGC1A |
| | | | | | NKX2-5 |
| | | | | | GATA4 |

TABLE 4b
Further exemplary conversions of the present invention. Source cell
types are shown in the far left column and target cell types in the top
row. The transcription factors required to covert the source cell type
to a cell that exhibits at least one characteristic of the
target cell type are shown.

| | Target cells | | | |
|---|---|---|---|---|
| | Endothelial | | | Epithelial |
| Source cells | cells | Astrocyte | Keratinocytes | cells |
| Dermal fibroblast | SOX17 | SOX2 | FOXQ1 | FOS |
| | SMAD1 | SOX9 | SOX9 | DBP |
| | TAL1 | ARNT2 | MAFB | HES1 |
| | IRF1 | SMAD1 | CDH1 | FOXA2 |
| | TCF7L1 | RUNX2 | FOS | ESRRA |
| | MXD4 | E2F5 | REL | CDH1 |
| | JUNB | PBX1 | | PAX6 |
| | | | | FOXQ1 |
| Keratinocyte | SOX17 | | | NOTCH1 |
| | TAL1 | | | HR |
| | SMAD1 | | | DBP |
| | IRF1 | | | OTX1 |
| | TCF7L1 | | | FOXQ1 |
| | HOXB7 | | | PAX6 |
| | | | | IRX5 |
| | | | | ESRRA |
| hESCs | SOX17 | IRF1 | SOX9 | MYC |
| | SMAD1 | SOX9 | NFKB1 | IL1B |
| | TAL1 | ARNT2 | MYC | FOS |
| | NFKB1 | PAX6 | FOSL2 | NFKB1 |
| | IRF1 | SNAI2 | NR2F2 | ESRRA |
| | HOXB7 | RUNX2 | FOSL1 | IRF1 |
| | JUNB | SOX5 | AHR | PAX6 |
| | | | | FOXQ1 |
| hiPSCs | SOX17 | PAX6 | TFAP2A | |
| | TAL1 | SNAI2 | MYC | |
| | NFKB1 | RUNX2 | SOX9 | |
| | IRF1 | HMGB2 | NFKB1 | |
| | SMAD1 | POU3F2 | TP63 | |
| | JUNB | SOX5 | NFKBIA | |
| | HOXB7 | E2F5 | | |
| Mesenchymal | | SOX2 | | |
| | | SOX9 | | |
| | | ARNT2 | | |
| | | MYBL2 | | |
| | | E2F1 | | |
| | | POU3F2 | | |
| | | HMGB2 | | |

The present invention includes the following non-limiting Examples.

### EXAMPLES

### Example 1

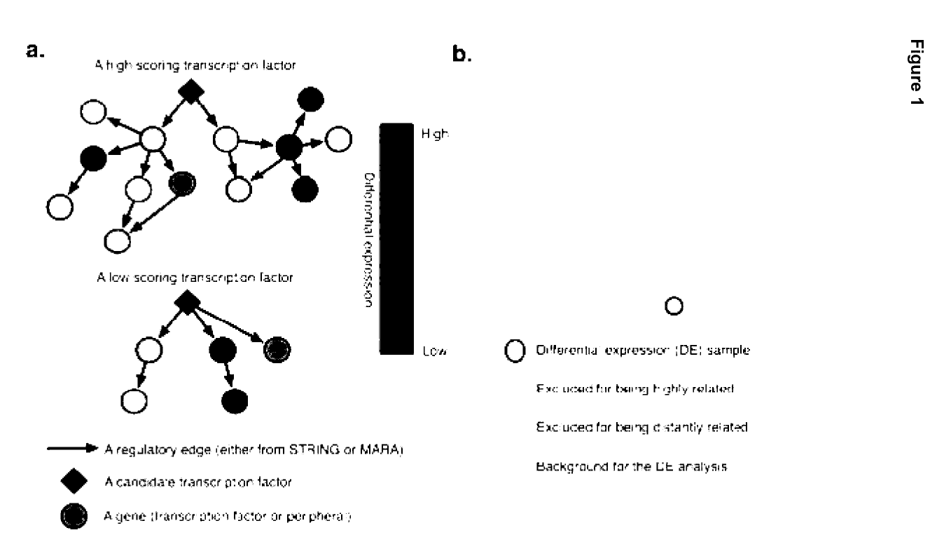

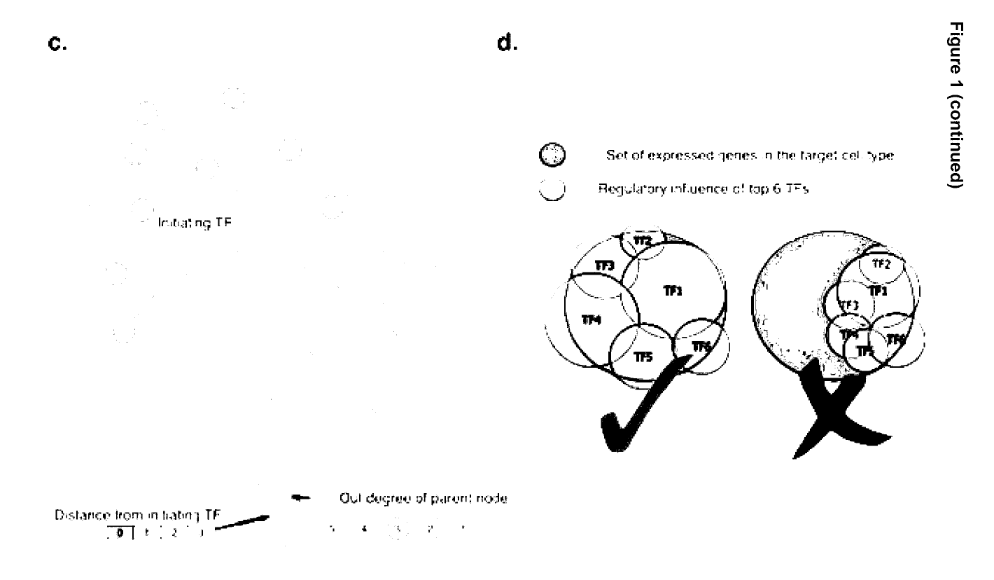

In order to predict the sets of TFs required for each cell conversion we identify those TFs that are not only differentially expressed between cell types, but also exert regulatory influence on other differentially expressed genes in the local network (see FIG. 1a). A single score that captures the differential expression for every gene in every cell type is defined by combining the log-fold change and adjusted p-value. The regulatory influence of each TF in each cell type is calculated by performing a weighted sum of the differential expression scores over the known interactome (as defined by STRING and MARA, see FIG. 1c). This sum is weighted by two factors: (1) by the directness of the regulation, i.e. how many intermediates between the TF and a downstream gene, and (2) the specificity, i.e. the number of other genes the upstream TF also regulates. This weighted sum allows TFs to be ranked in each cell type according to their influence. The final step is to select the optimal set of TFs with the greatest combined influence over genes differentially expressed in the target cell type compared to the source. This is done by adding TFs to the set in order of rank by differential influence, omitting those which don't increase the influence of the set, until the combined influence reaches 98% of expressed target cell genes (see FIG. 1d and methodology described below). Biologically speaking, Mogrify identifies TFs which control the parts of the regulatory network most responsible for the identity of the target cell type.

Mogrify may include one or more steps, which are outlined below and are described in more depth in the following sections:

1. Collect expression data for each gene (x) in each sample (s).

2. Calculate the differential expression against a tree-based background for each gene in each sample then combine the log fold change (Lₓ^{s}) and adjusted P− value (Pₓ^{s}) to a gene score (Gₓ^{s}).

3. For each TF (ₓ) in each sample calculate the network score (N^{S}) by performing a weighted sum of gene scores over two different sub networks (Kₓ_{^{MARA}}^{s} and Kₓ_{^{STRING}}^{s}) centered on each TF.

4. Rank TFs based on a combination of Gₓ^{s} and Nₓ^{s} scores.

5. Calculate the set of transcription factors for a conversion between any two cell types based on comparisons of ranked lists from each cell type.

6. Remove transcriptionally redundant TFs from the lists.

7. Create a cell conversion landscape by arranging the cell types on a 2D plane based on their required TFs and add a height based on the average coverage of the required genes that are directly regulated by the TFs selected.

Step 1: Expression Data Taken from FANTOM5 Dataset

Mogrify uses 700 libraries of clustered CAGE tags, which provide the TSS locations. These are mapped to their corresponding genes (provided by the FANTOM5 consortium (Forrest, A. R. R. et al. Nature 507, 462-470 (2014)). This data is used to create tag counts for each gene in each library. In total there are 15,878 distinct genes (of which 1408 are TFs) expressed with at least 20 TPM (tags per million) in at least one sample.

Step 2: Tree-Based Differential Expression

Calculating differential expression is a common problem when analyzing biological data and a number of techniques exist to do this. We elected to use DESeq for this work as it performs well in benchmark evaluations, it allows analysis of some non-replicated datasets and has a short runtime. In order to calculate differential expression, it is necessary to identify two groups: the set of samples you wish to identify differential expression in and the background to compare against. The problem of selecting the correct background is important. Too many irrelevant samples can reduce the statistical power of the test. Too narrow or too few samples in the background makes it impossible to tell which genes are truly differentially expressed. One solution is to perform an exhaustive calculation of pairwise tests between each of the cell types. This approach has two problems: firstly it is very computationally expensive and secondly it does not reveal the genes that are differentially expressed between a sample and an average background, but rather specifically between two samples. For Mogrify we are interested in the genes that are important for a given cell type in all situations and hence against a collection of samples. In order to do this we implemented a tree-based background selection method based on the FANTOM5 cell ontology (FIG. 1B). The principle of this approach is to exclude cell types whose ontologies are very close whilst including others that are near in the tree to the background. This was achieved by picking a point near to the top of the tree that would act as the breaking point. Samples in the same clade as the cell type being analyzed were removed and those not in the same clade, but still below this point, were included. The result of this is a set of samples that is broad enough to give reliable results but narrow enough that the statistical power is kept at a manageable level.

This tree-based background selection for DEseq is run on all FANTOM5 libraries (grouped by replicates) creating log-fold changes and FDR adjusted p-values for each gene in each sample. Because there is non-uniform background, the results of each differential expression calculation are not directly comparable, hence for the remaining steps, these figures are used to rank genes in each sample and it is the rankings that are compared.

Since we are only interested in identifying TFs with a high level of influence, we convert the log fold change and FDR adjusted p-values to a single positive score (G,$) using the following equation:

Gₓ^{s}=|Lₓ^{s}|(−log₁₀Pₓ^{s}) Equation 1:

Where
Lₓ^{s} is the log-fold change of gene x in sample s.
Pₓ^{s} is the adjusted p-value of gene x in sample s.

The formula ensures that those genes with high log-fold changes and a low adjusted p-value score very highly and vice versa. This is applied to every gene in each sample creating a 700 sample by 15878 genes matrix of differential expression.

Step 3: Calculate a TF's Network-Based Sphere of Influence

In order to assess the importance of each TF, its effect on its local neighborhood is calculated using two sources of network information: the STRING database and Motif Activity Response Analysis (MARA). These two techniques, described below, contain different types of interactions. MARA provides Protein-DNA interactions between TFs with known binding sites in the promoter regions of a gene. This represents a low-level directed regulatory network of interactions. STRING is a meta-database of interactions that contain various types of interactions including PROTEIN-PROTEIN, PROTEIN-DNA, PROTEIN-RNA as well as biological pathways. This provides a view of the interactions that takes place both directly and indirectly affecting gene expression.

In order to calculate the influence, a weighted sum of gene influences (from step 2) is performed over a transcription factor's local network neighborhood. This local network is constrained to a maximum of 3 edges and the effect of each node diminishes the further from the seed TF it is located and depending on the out degree of its parent (FIG. 1C). The distance weighting is used so that genes that are increasingly further from direct regulation have less of an impact on the score. The edge weighting is used to compensate for highly ubiquitous transcription factors and prevent them from receiving artificially high scores by regulating a large number of barely-differentially expressed genes. We consider that a TF that is regulating 10 genes that have Gₓ^{s}=100 to be more important than a TF that is regulating 1000 genes that have Gₓ^{s}=1.

The equation to perform this weighted sum is:

$${N}_{x , n}^{s} = {\sum }_{r ϵ {V}_{x}}^{s} {G}_{r}^{s} \cdot \frac{1}{{L}_{r , n}} \cdot \frac{1}{{O}_{r , n}} \qquad Equation 2$$

Where:
XϵVₓ is each gene (r) in the set of nodes (Vₓ) that make up the local sub-network of TF x.

L_{r,n} is the level (or number of steps) r is away from x in the network n.
O_{r,n} is the degree of the parent of r in the network n.

This is performed over both the MARA and STRING networks resulting in two TF-influence lists (N_{x,MARA}^{s} and N_{x,STRING}^{s}).

Step 4: Rank the TFs Based on the Results of Step 2 and 3

The result of steps 3 and 4 are three ranked TF lists for each sample based on Gₓ^{s}, N_{x,MARA}^{s} and N_{x,STRING}^{s}. To get the final ranking of each TF in each sample, its rank in each of the three lists is added together. Ranks are limited to a maximum of 100 as we observed that after the top 100 TFs the remaining regulatory influence was very small. If a TF doesn't appear in a particular list then it is given a score of 100. The result of this is a single ranked list of TFs for each cell type; those with the lowest score/rank are those predicted to facilitate a cell conversion.

Step 5: Compute all Pairwise Experiment Comparisons to Create Predictions

In order to predict the set of TFs for a given conversion the ranked lists from the source and target cell type are compared. If a TF from the target cell type list is already expressed in the source target (greater than 20 TPM) then it is removed from the list.

Step 6: Remove Transcriptionally Redundant TFs

Once the final ranking is complete, regulatory redundancy is removed. This is achieved by comparing the lists of genes that each of the TFs directly regulates. For a given TF, if there is a higher-ranking TF that regulates over 98% of the genes that it would regulate, then it is removed. This means that the resulting predictions include TFs that are diverse in their regulatory sphere of influence. This cutoff was chosen empirically to minimize the number of factors predicted whilst maximizing the network coverage (FIG. 5).

Step 7: Create a Cell Reprogramming Landscape Based on Steps 1-6

In order to create the reprogramming landscape we calculated the X and Y coordinates independently of the Z coordinate. In order to reduce the complexity of the landscape we average the gene expression profiles of individual samples grouped by the cell ontology provided by FANTOM5. The result of this is a set of 314 ontologies that contain at least three samples from which we have the average gene expression. The X and Y coordinates are calculated by doing a multi-dimensional scaling (MDS) of these profiles. The result of the MDS is a projection of the data where the distance between points is maintained from the multidimensional reality to two-dimensional reduction. As a result 2 points that are close together in the X-Y plane of the landscape have similar expression profiles and as such represent similar cell types. The Z-axis of the landscape is calculated by considering the regulatory coverage of the top 8 Mogrify predicted TFs. For every conversion we look at the set of genes that are expressed in the ontology and the number of these are directly regulated by each TF. We calculate the area under the curve of the cumulative coverage for the top 8 TFs normalised by the maximum possible AUC to retrieve a value between 0 and 1 for each ontology as the height. As such a height of 1 represents an ontology where all of the required genes are directly regulated by the top ranked TF and a height of 0 that none of the top 8 TFs directly regulate any of the required genes. The X, Y and Z values are then used in the R package plot3D in order to generate the landscape using the image2D and persp3D packages. The different stem cells at the highest locations were found with a gene set enrichment score of 0.41 and a p-value of 0.011.

### Example 2

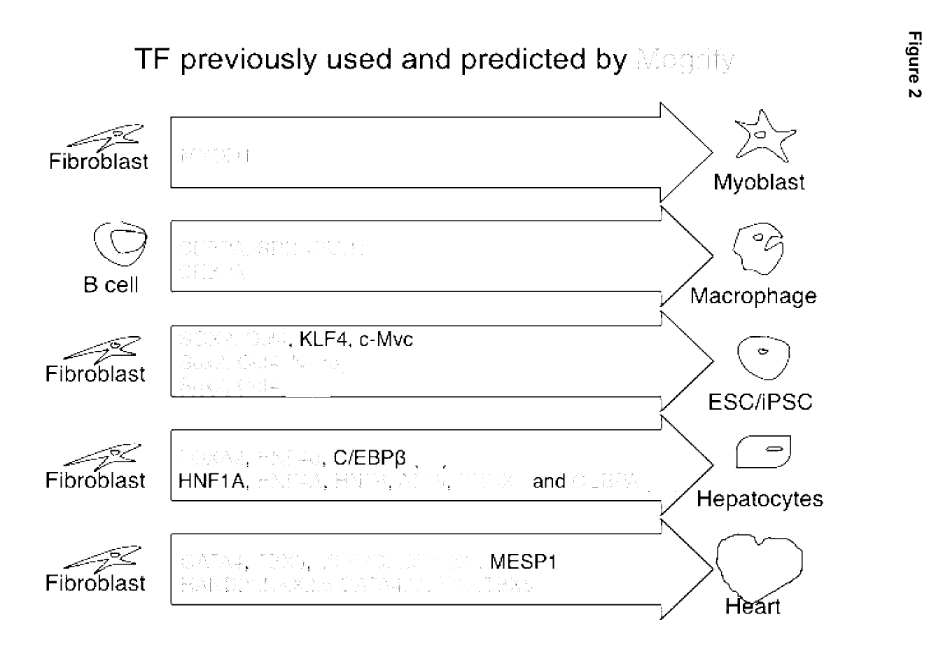

In order to assess the predictive power of Mogrify we first determined how Mogrify performs against well-known, previously published direct cell conversions, focusing on those involving human cells. These should not be considered as absolute perfect combinations, but as positive example reference points useful for comparison. As shown in FIG. 2, in almost every case Mogrify predicts the complete set of TFs previously demonstrated to work, but sometimes includes an upstream TF in lieu of the published factor. For example, it is known that human fibroblasts can be converted to iPS cells by introducing OCT4 (also known as POU5F1), SOX2, KLF4 and MYC or OCT4, SOX2, NANOG and LIN28. Mogrify predicts NANOG, OCT4 and SOX2 as the top 3 TFs for this conversion, a combination that has also been experimentally validated. Previous work has demonstrated that the conversion of B-cells and fibroblasts into macrophage-like cells was possible by the expression of CEBPa and PU.1 (also known as SPI1) (Xie, H., Ye, M., Feng, R. & Graf, T. Cell 117, 663-676 (2004); Rapino, F. et al. Cell Rep. 3, 1153-63 (2013) which Mogrify perfectly predicts. For the conversion of human dermal fibroblasts into cardiomyocytes, we chose to not use the data in the FANTOM5 set since it lacks many key cardiomyocyte genes (indicating a deficiency in the origin of the sample). Nevertheless using the heart sample, which is a cellularly heterogeneous tissue and not ideal, Mogrify's predicted list includes four out of the five TFs (or a closely related factor) used in the human conversion (Fu, J.-D. et al. Stem cell reports 1, 235-47 (2013)). There are a number of reports in the literature of transdifferentiations from various cell types to neurons in both mouse and human (Table 5).

TABLE 5
Transitions resulting in neuronal phenotypes. In each case, the set of
transcription factors used to convert the source cell type to the
target cell type are shown.

| | Target Cell | TFs used for |
|---|---|---|
| Source Cell Type | Type | reprogramming |
| Fibroblasts | Neurons | ASCL1, BRN2 and |
| | | MYT1L |
| Human Fibroblasts | Neurons | ASCL1, BRN2, MYT1L |
| | | and NEUROD1 |
| Human Fibroblasts | Neurons | miR-9/9-124, NEUROD2, |
| | | ASCL1 and MYT1L |
| Human Fibroblasts | Neurons | miR-124, MYT1L and |
| | | BRN2 |
| Fibroblasts | Dopaminergic | ASCL1, BRN2, MYT1L, |
| | Neurons | LMX1A and FOXA2 |
| Astrocytes | Dopaminergic | ASCL1, LMX1B and |
| | Neurons | NURR1 |
| Fibroblasts | Dopaminergic | ASCL1, NURR1 and |
| | Neurons | LMX1A |

The sets of TFs used vary, probably due to the heterogeneity and complexity of neurons, however factors common to all experiments are predicted by Mogrify (Table 6).

TABLE 6
The Mogrify predictions for transdifferentiation between human dermal
fibroblasts and neurons. The TFs are ranked according to their
Mogrify score and those shown in italics are those selected by Mogrify
as not being redundant to other higher-ranking TFs.

| | Source | |
|---|---|---|
| TF name | TPM | Target TPM |
| CUX2 | 0 | 20 |
| SOX2 | 0 | 100 |
| NEUROD1 | 0 | 19 |
| NEUROG2 | 0 | 23 |
| HES6 | 0 | 67 |
| FOXG1 | 0 | 266 |
| ASCL1 | 0 | 22 |
| SOX9 | 0 | 45 |
| ZNF238 | 0 | 177 |
| NEUROD2 | 0 | 238 |
| NEUROD6 | 0 | 162 |
| ACTL6B | 0 | 37 |
| MYT1L | 0 | 37 |
| POU3F2 | 0 | 42 |
| SCRT2 | 0 | 36 |

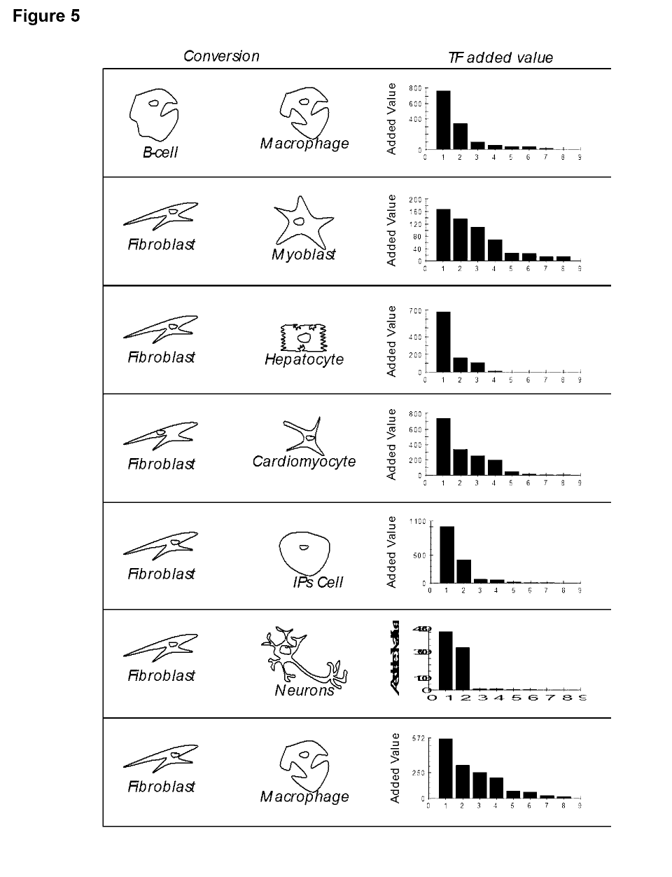

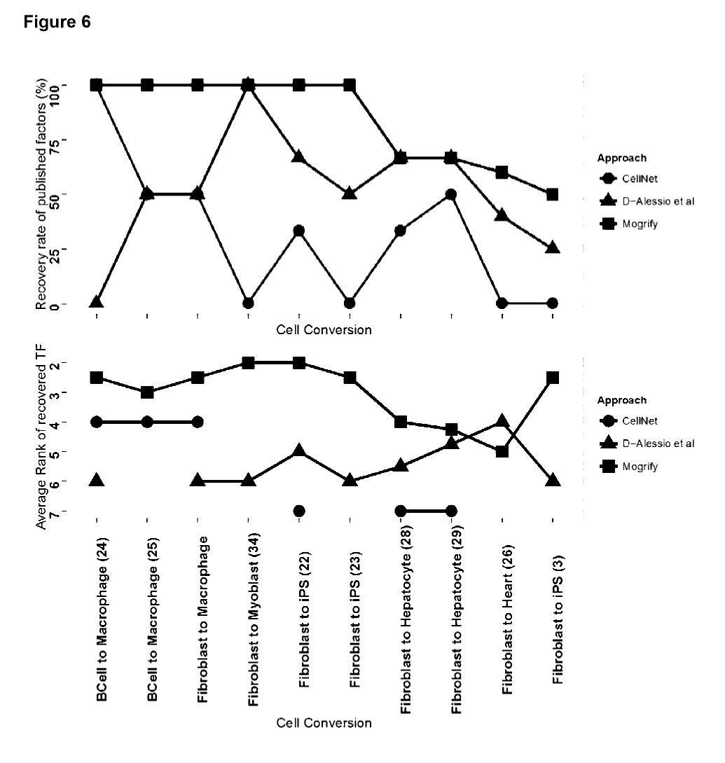

Finally between human fibroblasts and hepatocytes, Mogrify predicts a combination of TFs highly similar to that required for conversion and maturation (FIG. 2). Using the conversions shown in FIG. 2 we assessed the ability of Mogrify, CellNet and the entropy-based approach from D'Alessio et al (Stem Cell Reports, Volume 5, Issue 5, 10 Nov. 2015, Pages 763-775) to recover these known factors. The average recovery rate of the published transcription factors for Mogrify was 84%, for CellNet 31% and D'Alessio et al 51% (FIG. 6). In six out of the ten conversions in FIG. 2 Mogrify recovered 100% of the required TFs, meaning that if Mogrify had been used to provide the TF set for these conversions, the experiment could have been a success first time. On the other hand CellNet and D'Allesio et al only recovered all factors for one of the ten conversions.

Having mapped the landscape of human cell type in terms of naturally-occurring states and the transitions between them, a core control set of TFs that describe the individual cell types is captured, even though the primary aim of Mogrify is to predict TFs for cellular conversions. It is believed that this per se could aid researchers to unveil the role of different TFs in their favourite cell type. In practice Mogrify provides a significant advance over the strategies currently being applied in laboratories for cell reprogramming, helping in the prediction of TFs whose over-expression will induce directed cell conversion. Mogrify has been pre-calculated on conversions between all possible combinations of the 307 FANTOM5 tissue/cell types resulting in 93,942 directed conversions. Mogrify could be applied to many other cell types not included in FANTOM5 if the expression signature (e.g. RNAseq or CAGE) is known. Mogrify provides a starting point and systematic means to explore new conversions in human. Because Mogrify incorporates a TF redundancy step, it is able to give a finite set of TFs as a prediction for the cell conversion, which is of more utility than just the ranking of all TFs.

In order to compare the performance of Mogrify with other methods a benchmarking experiment was carried out. Firstly, to assess the effect on performance of using the complete Mogrify algorithm in comparison to using the MARA, STRING and differential expression components alone. Secondly a comparison with CellNet and D'Allesio et al. was carried out. These are the only other techniques that currently provide a means to calculate transcription factor sets for a wide variety of cell types. In order to carry out a comparison the sets of transcription factors from the published conversions shown in FIG. 2 were used as true positives. The benchmark consisted of assessing the performance of each technique in recovering these TFs using the following steps:
1) For each conversion identify the number of transcription factors to consider: Mogrify is the only method to provide a set of TFs rather than a ranked list of all TFs, and since the object is to compare other methods to Mogrify, the information generated by Mogrify on the number of factors to use was shared to the other methods, i.e. no method is allowed to use more factors than the other methods. For example for the conversion between B-Cell and macrophage, Mogrify predicts that 8 TFs should be adequate, so for all methods the top 8 TFs are used for comparison.
2) For each method identify if the correct transcription factors have been predicted: For each published set of transcription factors the predictions from each method are compared and two statistics extracted. Firstly the recovery rate of the published transcription factors (i.e. 100% if all of the published factors were contained in the predicted set) and secondly the average rank of the published factors (i.e. for each correctly identified TF the ranks are summed and divided by the total number of correctly identified TFs).

The results from these two steps can be found in Tables 7 and 8 and a summary of the comparison of Mogrify to CellNet and D'Allesio et al. can be found in FIG. 6.

In order to extract the results for CellNet we used publicly available datasets for fibroblasts (GSE14897) and B-Cells (GSE65136) as the starting point and used the web interface to CellNet (cellnet.hms.harvard.edu) to provide predictions for each of the conversions in FIG. 2. D'Allessio et al. provide ranked sets of TFs for many cells types and these ranked lists were used for the comparison.

### Example 3

In order to empirically demonstrate the predictive capabilities of Mogrify we conducted 11 novel cell conversions using human cells:
fibroblasts to keratinocytes (results in Example 4);
keratinocytes to endothelial cells (results in Example 5);
fibroblasts to endothelial cells (results in Example 6);
embryonic stem cells to endothelial cells (results in Example 7);
induced pluripotent stem cells to endothelial cells (results in Example 8);
fibroblasts to astrocytes (results in Example 9);
embryonic stem cells to astrocytes (results in Example 10);
induced pluripotent stem cells to astrocytes (results in Example 11);
bone mesenchymal stem cells to astrocytes (results in Example 12);
embryonic stem cells to keratinocytes (results in Example 13); and
induced pluripotent stem cells to keratinocytes (results in Example 14).

The materials and methods are described in this example.

Lentiviral Generation

For lentiviral generation, 293T human embryonic kidney (HEK; Sigma) cells were cultured in T-75 flasks. Once they reached 90-95% confluence, they were transfected with a -Iv165 vector expressing relevant transcription factors (for example CDH1, FOS, FOXQ1, HOXB6, IRF1, MAFB, REL, SMAD1, SOX9, SOX17, TAL1, TCF7L1, MXD4, NFKB1, SOX2, ARNT2, RUNX2, PAX6, SNAI2, HMGB2, E2F1, MYC, FOSL2, or TFAP2A,) from the EF1alpha promoter and IRES2-eGFP (GeneCopoeia), together with second generation Trono lab packaging plasmids psPAX2 and pMD2.G (Addgene) using LTX lipofectamine (Invitrogen) transfection agent. Viral supernatants were collected at 24 hrs and 36 hrs post transfection and concentrated with ultra-centrifugal filters (Millipore). Viral concentrates were then stored at −80° C. Titrations were based on eGFP expression as determined by flow cytometry. The cell line used in these experiments tested negative for mycoplasma contamination.

Cell Culture

Prior to their use in experiments, human adult epidermal keratinocytes (HEKa; GIBCO) and human dermal fibroblasts (HDFs; GIBCO) were expanded at 2.5×10³ cells/cm² and passaged at least 3 times. HEKa cells were cultured in Keratinocyte serum free media (KSFM; GIBCO) which contained 10% HKGS (GIBCO) and 1% Pen/Strep (GIBCO). HDFs, on the other hand, were cultured in medium 106 (GIBCO) which contained 10% LSGS (GIBCO) and 1% Pen/Strep. Cells were then frozen in liquid nitrogen for later use. For keratinocyte to endothelial cell transdifferentiation, cells were thawed and seeded at 2.5×10³ cells/cm² until they reached 90% confluence. They were then reseeded at 5.0×10³ cells/cm² for two days in KSFM media, before being infected with concentrated lentiviral particles of HOXB6, IRF1, SMAD1, SOX17, TAL1, and TCF7L1 in presence of polybrene (Millipore) in KSFM media. After the addition of viruses (12-24 hrs), media was replaced with fresh KSFM media. At day 4, media was replaced with human endothelial serum free media (GIBCO) with 1% Pen/Strep containing human VEGF (50 ng/μl; PeproTech), human BMP4 (20 ng/μl; PeproTech) and human FGF2 (20 ng/μl; PeproTech). For fibroblast to keratinocyte transdifferentiation, cells were seeded at 2.5×103 cells/cm2 until they reached 90% confluence. They were then reseeded at 2.5×103 cells/cm2 for 24 hrs in mouse fibroblast media (MEFM), before being transduced with the lentiviral particles of CDH1, FOS, FOXQ1, MAFB, REL, and SOX9 in presence of polybrene in MEFM for 24 hrs. At day 4, media was replaced with KSFM media containing 1% Pen/Strep, retinoic acid and human BMP4 (R&D). Fresh media was added at least once every two days throughout all of the experiments. Each of those experiments was repeated 3-4 times.

TABLE 9
Cell culture media that can be used to culture other cell types
are shown in the following table.

| Cell | Media | Cat#: | Company |
|---|---|---|---|
| Astrocytes | Astrocyte Medium | A1261301 | Life |
| | | | Technologies |
| Dermal | Medium106 | M-106-500 | ThermoFisher |
| fibroblasts | | | |
| Endothelial cells | Medium 131 | M131500 | Life |
| | | | Technologies |
| Epidermal | EpiLife | M-EPICF-500 | ThermoFisher |
| Keratinocytes | | | |
| H9 ESC line | KSR | 10828-028 | ThermoFisher |
| | Essential 8 | A1517001 | Life |
| | | | Technologies |
| Monocytes | Macrophage-SFM | 12065-074 | ThermoFisher |
| Chondrocytes | Eagle's Minimum | 10-009-CV | Corning |
| | Essential Medium | | |
| Hair Follicles | Medium 199/Ham's | 11150- | ThermoFisher |
| | F12 | 059/11765-047 | |
| CD4+ T-cell | CTS ™ | A10485-01 | ThermoFisher |
| | OpTmizer ™ | | |
| | T Cell Expansion | | |
| | SFM | | |
| CD8+ T-cell | CTS ™ | A10485-01 | ThermoFisher |
| | OpTmizer ™ | | |
| | T Cell Expansion | | |
| | SFM | | |
| NK-cell | alpha MEM | M 8042 | Sigma Aldrich |
| PSCs | Essential 8 Medium | A1517001 | Life |
| | | | Technologies |
| HSCs | StemPro ® CD34+ | A14059 | ThermoFisher |
| | Cell Kit | | |
| MSCs of adipose | StemPro ® Human | R7788-110 | ThermoFisher |
| | Adipose-Derived | | |
| | Stem Cell Kit | | |
| MSCs of bone | StemPro ® BM | A15652 | ThermoFisher |
| marrow | Mesenchymal | | Life |
| | Stem Cells kit | | Technologies |
| | Alpha-MEM with | | |
| | 15% FBS, | | |
| | glutamine, penicillin | | |
| | ands treptomycin | | |
| Oligodendrocytes | Neurobasal | 21103-049 | ThermoFisher |
| precursors | medium | | |
| Skeletal muscle | DMEM | 11965-092 | ThermoFisher |
| cells | | | |
| Smooth muscle | Medium 231 | M-231-500 | ThermoFisher |
| cells | | | |

Flow Cytometry

At various time-points, transdifferentiating cells were dissociated with 0.25% trypsin-EDTA (GIBCO) for 3 minutes at 37° C. Cells were then prepared for flow cytometric analysis or sorting. They were incubated with anti-human CD31-APC (17-0319-41, eBioscience) at 4° C. for 15 minutes, washed with DPBS (GIBCO), centrifuged at 1000 rpm for 7 minutes then resuspended in propidium iodide (Sigma-Aldrich) containing media. A LSR-II analyser (BD Bioscience) and the Influx cell sorter (BD Biosciences) were used for data analysis and sorting respectively.

qPCR

Total RNA was extracted using the RNeasy Micro Kit (Qiagen) following the manufacturer's instructions. Extracted RNA was reverse transcribed into cDNA using a Superscript III kit (Invitrogen). Real-time quantitative PCR reactions were set up in triplicate using a Brilliant II SYBR Green QPCR Master Mix (Stratagene) and then run on 7500 Real time PCR System. Primer sequences for qPCR are:

F-CD31:
CCTTCTGCTCTGTTCAAGCC
R-CD31:
GGGTCAGGTTCTTCCCATTT
F-VE:
ATGAGAATGACAATGCCCCG
R-VE:
TGTCTATTGCGGAGATCTGCAG
F-VEGFR2:
GGCCCAATAATCAGAGTGGCA
R-VEGFR2:
CCAGTGTCATTTCCGATCACTTT
F-KERATIN1:
AGAGTGGACCAACTGAAGAGT
R-KERATIN1:
ATTCTCTGCATTTGTCCGCTT
F-KERATIN14:
AGACCAAAGGTCGCTACTGC
R-KERATIN14:
AGGAGAACTGGGAGGAGGAG
F-INVOLUCRIN:
CTGCCTCAGCCTTACTGTGA
R-INVOLUCRIN:
GGAGGAGGAACAGTCTTGAGG
F-β-ACTIN:
CATGTACGTTGCTATCCAGGC
R-β-ACTIN:
CTCCTTAATGTCACGCACGAT

Immunofluorescence

Cells were fixed with 4% paraformaldehyde in DPBS at room temperature for 10 minutes. There was no need to permeabilise the cells as the markers of interest are expressed on the cell surface. Cells were blocked with 5% donkey serum in DPBS for 30 minutes and then incubated with primary antibodies (goat polyclonal anti CD31, sc-1506; Santa Cruz; and rabbit polyclonal anti VE-Cadherin, ab33168; abcam) overnight at 4° C. The next day, cells were incubated with secondary antibodies (donkey anti goat Alexa Flour-555; Invitrogen, and donkey anti rabbit Alexa Flour-647; Invitrogen) for two hours at room temperature. Finally, cells were overlayed with 4′,6-diamidino-2-phenylindole (DAPI; Life Technologies) for 1 minute. All images were taken using the inverted Nikon Eclipse Ti epifluorescence microscope with Nikon Digital sight DS-U2 camera, and were processed and analysed using FIJI software.

### Example 4—Human Fibroblast to Keratinocyte (iKer) Conversion

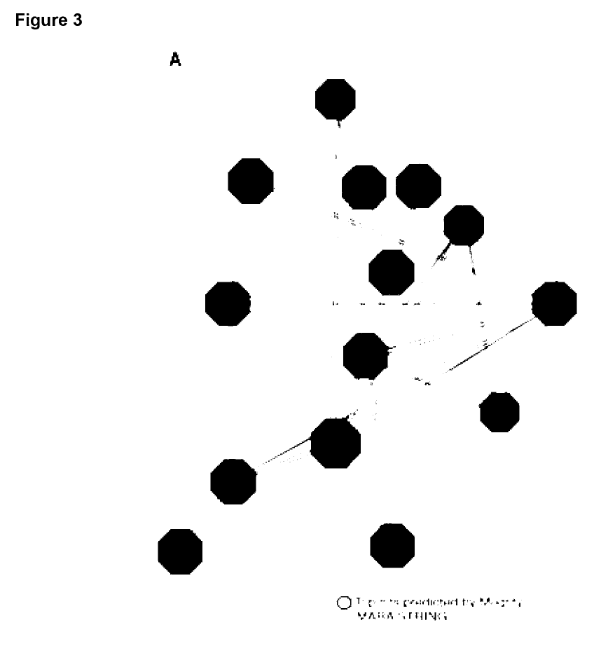

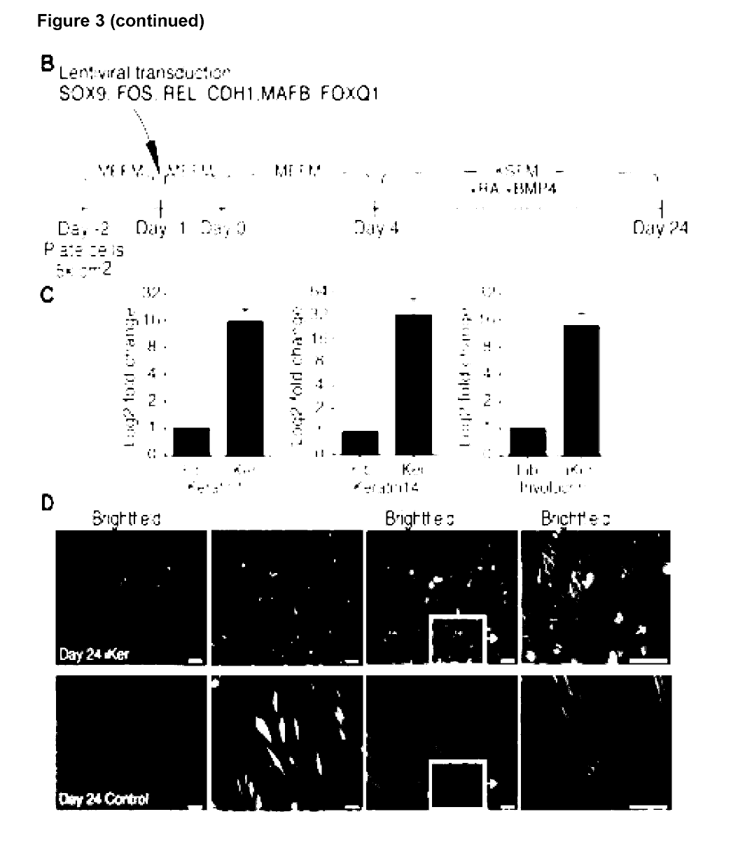

For this conversion, cells were transduced with FOXQ1, SOX9, MAFB, CDH1, FOS and REL, predicted by Mogrify (FIG. 3A and Table 10).

TABLE 10
The Mogrify predictions for transdifferentiation between human dermal
fibroblasts and Keratinocytes. The order of the table denotes the
original ranking and those in italics are those selected by Mogrify
as being the non-redundant set that should be used for reprogramming.

| TF name | Source TPM | Target TPM |
|---|---|---|
| SOX9 | 0 | 116 |
| CDH1 | 0 | 372 |
| TP63 | 0 | 82 |
| IRF6 | 0 | 374 |
| TFAP2A | 0 | 200 |
| SOX15 | 0 | 326 |
| CITED4 | 30 | 224 |
| HR | 0 | 41 |
| KLF5 | 0 | 170 |
| TRIM29 | 0 | 359 |
| AFAP1L2 | 0 | 39 |
| NRG1 | 17 | 144 |
| EHF | 0 | 63 |
| GCLC | 0 | 98 |
| PPP1R13L | 0 | 145 |
| MXD1 | 0 | 19 |
| TNFRSF10A | 10 | 38 |
| INHBA | 0 | 158 |
| HES2 | 0 | 83 |
| ZNF219 | 30 | 96 |
| BNC1 | 16 | 104 |
| FST | 89 | 340 |
| TRIB3 | 68 | 344 |
| FOS | 32 | 58 |
| GRHL3 | 0 | 30 |
| CORO2A | 0 | 16 |
| HOXA1 | 0 | 13 |
| TRAK1 | 0 | 25 |
| ETV4 | 57 | 94 |
| PIM1 | 11 | 21 |
| REL | 0 | 12 |
| TNF | 0 | 10 |
| MAFB | 0 | 17 |
| FOXQ1 | 0 | 26 |
| NOTCH1 | 0 | 83 |
| TFCP2L1 | 0 | 18 |
| OTX1 | 0 | 10 |
| GRHL2 | 0 | 19 |
| CTNNBIP1 | 17 | 99 |
| IRAK2 | 0 | 10 |

By day 16 post-transduction, keratinocyte-associated markers keratin1, keratin14 and involucrin, were markedly up-regulated in the transdifferentiated cells (FIG. 3C). Moreover, within three weeks, the majority of transduced cells exhibited cobblestone morphology, a classic characteristic displayed by keratinocytes. Adjacent un-transduced GFP negative cells or control cells transduced with GFP-only viruses maintained their fibroblastic morphology (arrow in FIG. 3D). This morphological and molecular characterization of the reprogrammed cells indicates that Mogrify successfully predicts the TFs necessary to induce the conversion from human fibroblasts to keratinocyte-like cells.

### Example 5—Adult Human Keratinocyte (HEKa) to Microvascular Endothelial Cells (iECs)

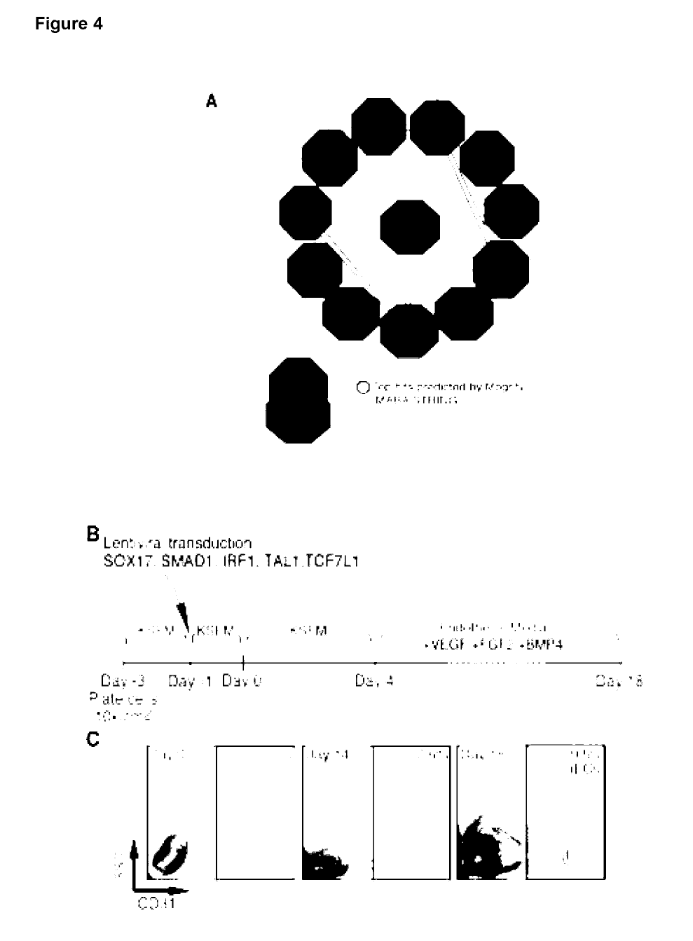

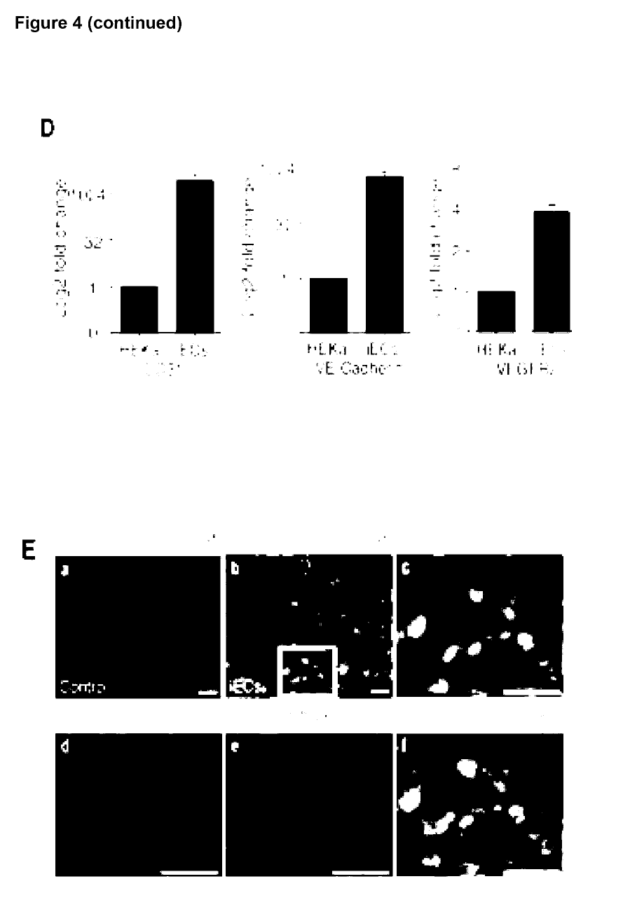

For this conversion we selected SOX17, TAL1, SMAD1, IRF1 and TCF7L1 to be used from the six TFs suggested by Mogrify (FIG. 4 and Table 11).

TABLE 11
The Mogrify predictions for transdifferentiation between human
Keratinocytes and Microvascular Endothelial Cells. The order of
the table denotes the original ranking and those in italics are those
selected by Mogrify as being the non- redundant set that
should be used for reprogramming.

| TF name | Source TPM | Target TPM |
|---|---|---|
| SOX17 | 0 | 317 |
| SMAD1 | 20 | 141 |
| TAL1 | 0 | 26 |
| SOX7 | 19 | 164 |
| ACVRL1 | 0 | 170 |
| HOXB7 | 0 | 34 |
| HOXD9 | 0 | 29 |
| FABP4 | 0 | 1594 |
| HOXD1 | 0 | 51 |
| HHEX | 0 | 114 |
| BCL6B | 0 | 100 |
| LDB2 | 0 | 126 |
| SOX18 | 0 | 613 |
| ERG | 0 | 147 |
| CYTL1 | 0 | 167 |
| ARRB1 | 0 | 213 |
| ANKRD1 | 0 | 454 |
| HOXD8 | 0 | 33 |
| PIR | 17 | 149 |
| EPAS1 | 113 | 760 |
| MXD4 | 42 | 202 |
| KLF2 | 0 | 51 |
| ABCG1 | 0 | 81 |
| IRF1 | 13 | 67 |
| TCF7L1 | 11 | 32 |
| NFKBIA | 49 | 106 |
| SOX4 | 53 | 228 |
| ESX1 | 0 | 18 |
| ID2 | 0 | 20 |
| PROX1 | 0 | 17 |
| AEBP1 | 0 | 28 |
| INSR | 0 | 24 |
| TNFSF4 | 0 | 22 |
| WWTR1 | 26 | 248 |
| NFKB1 | 27 | 44 |
| SP6 | 0 | 14 |
| HOXB6 | 0 | 15 |
| NFE2L3 | 0 | 32 |
| IGSF1 | 0 | 17 |
| FGF2 | 0 | 16 |
| SMAD9 | 0 | 13 |
| PDLIM1 | 449 | 847 |
| ZNF71 | 0 | 34 |
| BCL3 | 21 | 31 |
| ZNF267 | 0 | 16 |

These five TFs are predicted to regulate ˜92% of the required genes for iECs. Once these TFs were over-expressed in the HEKa cells we determined that the cells needed to be kept in their media until day four (FIG. 4B). We used FACS to follow the kinetics of the cell reprogramming, using the well-established endothelial marker CD31 (FIG. 4C), and by day 14 after transduction we detected that more than 2% of the infected cells had up-regulated CD31 and by day 18 almost 10% had up-regulated CD31. At that point we isolated those CD31 cells and evaluated the expression of the endothelial-associated genes (CD31, VE-Cadherin, and VEGFR2) by qPCR which resulted in a clear reactivation of all the assessed genes (FIG. 4D). Finally, we performed immunofluorescence (IF) to verify the morphology and expression of the trans-differentiated cells. As shown in FIG. 4E, only the cells transduced with the predicted TFs—and not the control cells-presented the right morphology and expressed CD31 and VE—Cadherin on the surface. This morphology and molecular characterization of the reprogrammed cells indicates the successful transition of human keratinocytes into human endothelial-like cells.

### Example 6—Fibroblast to Endothelial Cell

Transcription Factors used: SOX17, SMAD1, TAL1, IRF1, TCF7L1 and MXD4. (Mogrify also identified the factor JUNB but this was not used).

Transdifferentiation strategy: Human Dermal Fibroblasts were seeded onto well plates at 5k cells/cm² 24 hours prior to viral transduction of transcription factors in medium 106 with LSGS (Life Technologies). On the following day, lentiviral particles encoding the transcription factors were transduced to cells in Medium 106 with Polybrene (Merck Millipore). Well plates were then centrifuged at 1900 rpm for 60 minutes immediately after transduction. At day 5, medium was replaced with endothelial medium (Medium 131, Life Technologies) supplemented with VEGF (50 ng/ml, Miltenyi Biotec), FGF2 (20 ng/ml, Miltenyi Biotec), and BMP4 (20 ng/ml, Miltenyi Biotec). Medium was changed every 2 days throughout the experiment.

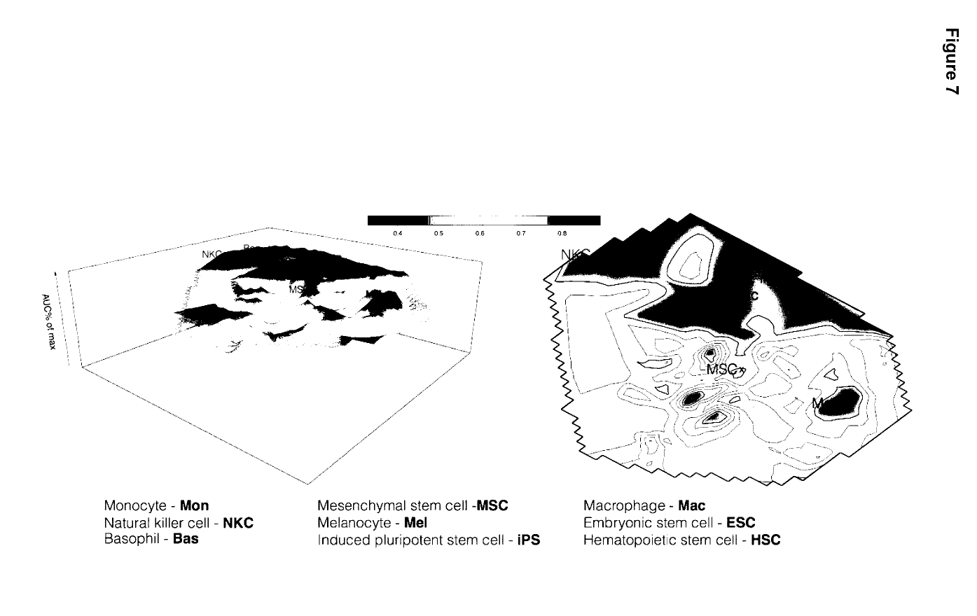

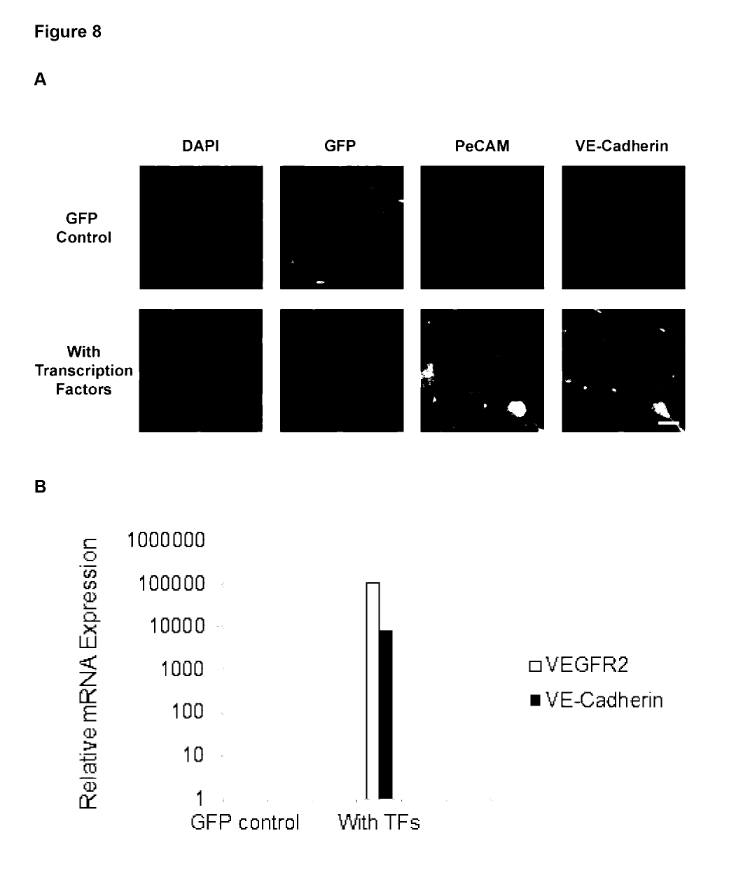

Immunofluorescence analysis showed evidence of expression of the endothelial markers PeCAM and VE-cadehrin at day 18 of transdifferentiation (FIG. 8A).

qPCR analysis also showed expression levels of the endothelial associated genes VEGFR2 and VE-Cadherin at day 18 of transdifferentiation (FIG. 8B).

### Example 7—Embryonic Stem Cell to Endothelial Cell

Transcription Factors used: SOX17, SMAD1, TAL1, NFKB1 and IRF1. (Mogrify also identified the factors HOXB7 and JUNB but these were not used).

Transdifferentiation strategy: Human Embryonic Stem Cells (H9) was seeded onto matrigel-coated (BD Falcon) well plates at 5k cells/cm² 24 hours prior to viral transduction of transcription factors in Essential 8 medium (Life Technologies). On the following day, lentiviral particles encoding the transcription factors were transduced to cells in Essential 8 medium with Polybrene (Merck Millipore). Well plates were then centrifuged at 1900 rpm for 60 minutes immediately after transduction. At day 5, medium was replaced with endothelial medium (Medium 131, Life Technologies) supplemented with VEGF (50 ng/ml, Miltenyi Biotec), FGF2 (20 ng/ml, Miltenyi Biotec), and BMP4 (20 ng/ml, Miltenyi Biotec). Medium was changed every 2 days throughout the experiment.

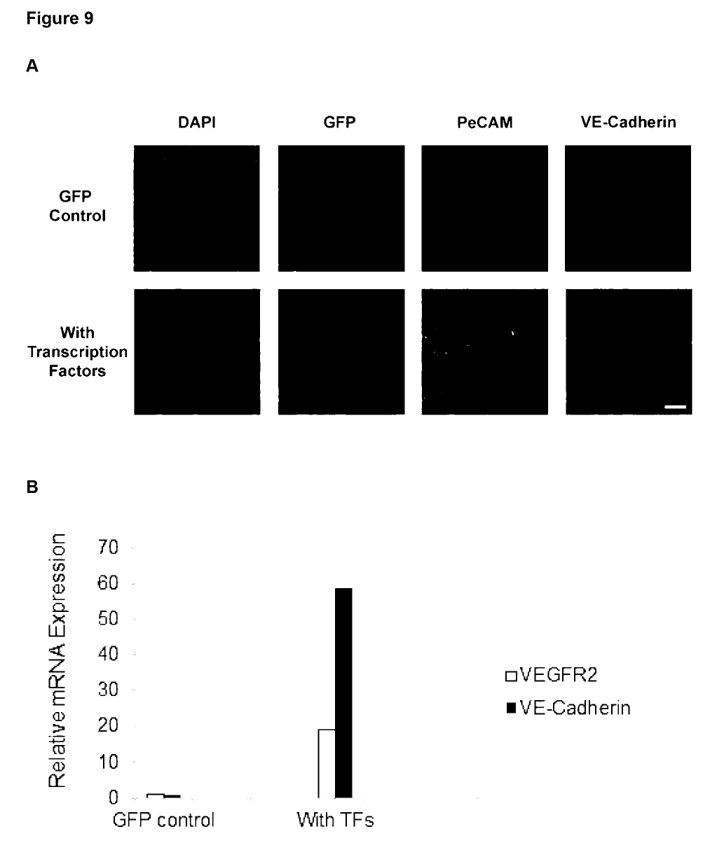

Immunofluorescence analysis showed expression of the endothelial markers PeCAM and VE-cadehrin at day 18 of transdifferentiation (FIG. 9A).

qPCR analysis showed expression levels of the endothelial associated genes VEGFR2 and VE-Cadherin at day 18 of transdifferentiation (FIG. 9B).

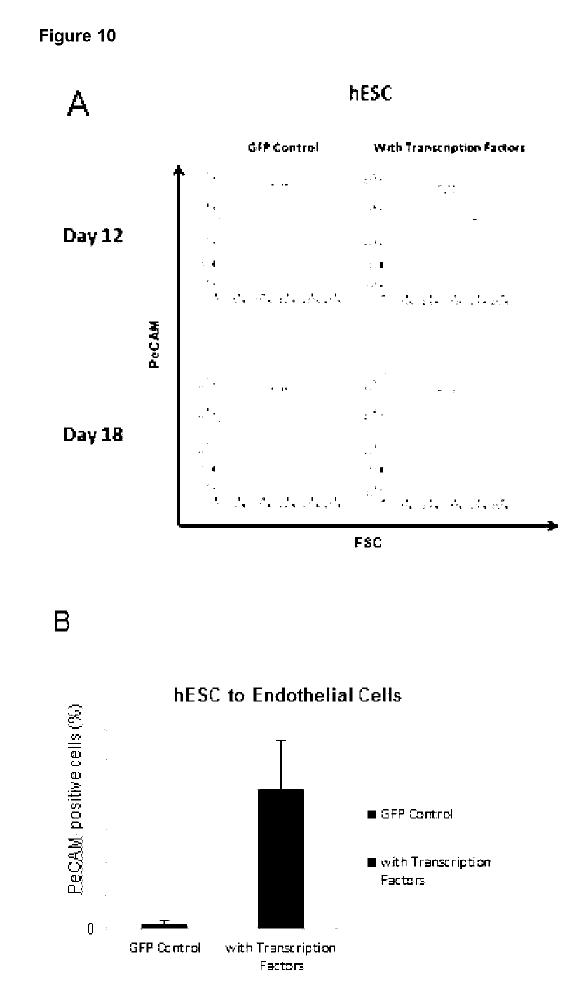

FIG. 10 shows the results of flow cytometry analysis of PeCAM expression at day 12 and 18 of transdifferentiation and quantification of PeCAM-positive cells at day 18 of transdifferentiation.

### Example 8—Pluripotent Stem Cell to Endothelial Cell

Transcription Factors used: SOX17, TAL1, NFKB1, IRF1, and SMAD1. (Mogrify also identified the factors HOXB7 and JUNB but these were not used).

Transdifferentiation strategy: Human Induced Pluripotent Stem Cells (32F donor) was seeded onto matrigel-coated (BD Falcon) well plates at 5k cells/cm² 24 hours prior to viral transduction of transcription factors in Essential 8 medium (Life Technologies). On the following day, lentiviral particles encoding the transcription factors were transduced to cells in Essential 8 medium with Polybrene (Merck Millipore). Well plates were then centrifuged at 1900 rpm for 60 minutes immediately after transduction. At day 5, medium was replaced with endothelial medium (Medium 131, Life Technologies) supplemented with VEGF (50 ng/ml, Miltenyi Biotec), FGF2 (20 ng/ml, Miltenyi Biotec), and BMP4 (20 ng/ml, Miltenyi Biotec). Medium was changed every 2 days throughout the experiment.

Immunofluorescence analysis shows expression of endothelial markers PeCAM and VE-cadehrin at day 18 of transdifferentiation (FIG. 11A).

qPCR analysis shows expression levels of the endothelial associated genes VEGFR2 and VE-Cadherin at day 18 of transdifferentiation (FIG. 11B).

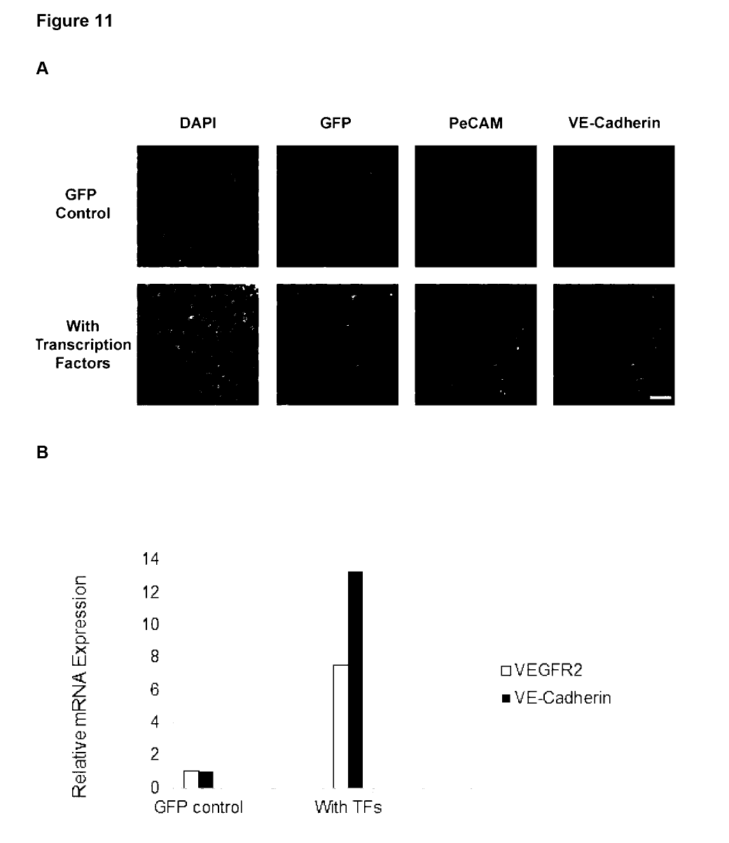

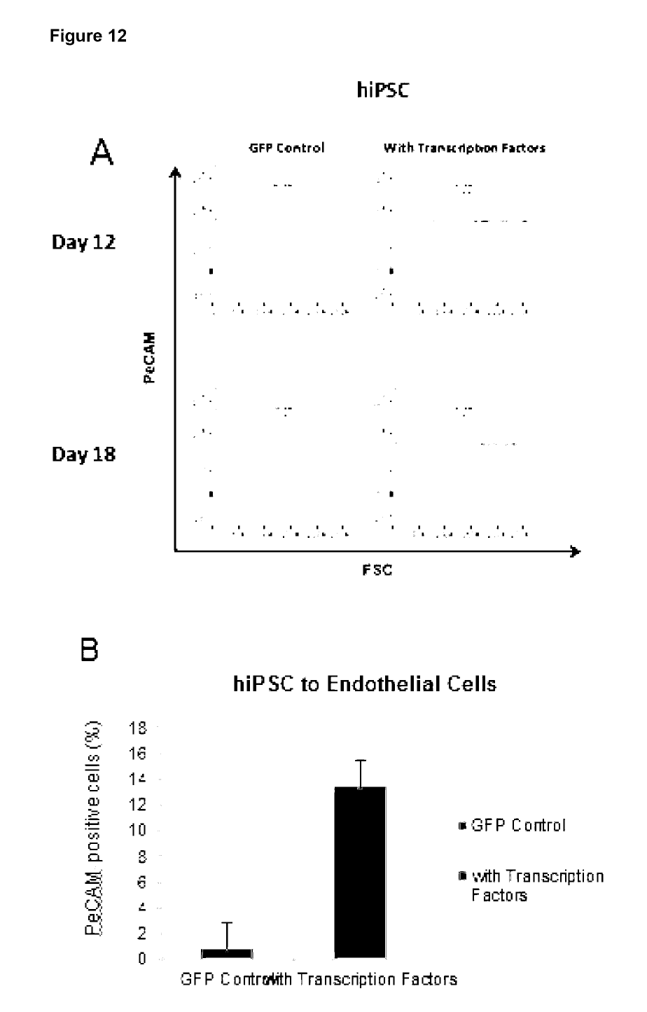

FIG. 12 shows flow cytometry analysis of PeCAM expression at day 12 and 18 of transdifferentiation. FSC, forward scatter and quantification of PeCAM-positive cells at day 18 of transdifferentiation.

### Example 9—Fibroblast to Astrocyte

Transcription Factors used: SOX2, SOX9 ARNT2, SMAD1 and RUNX2. (Mogrify also identified the factor E2F5 and PBX1 but these were not used).

Transdifferentiation strategy: Human Dermal Fibroblasts was seeded onto well plates at 5k cells/cm² 24 hours prior to viral transduction of transcription factors in medium 106 with LSGS (Life Technologies). On the following day, lentiviral particles encoding the transcription factors were transduced to cells in medium 106 with Polybrene (Merck Millipore). Well plates were then centrifuged at 1900 rpm for 60 minutes immediately after transduction. At day 5, medium was replaced with astrocyte medium (Life Technologies) supplemented with IL1β (10 ng/ml, Sigma-Aldrich). At day 7, medium was replaced with astrocyte medium. Medium was changed every 2 days throughout the experiment.

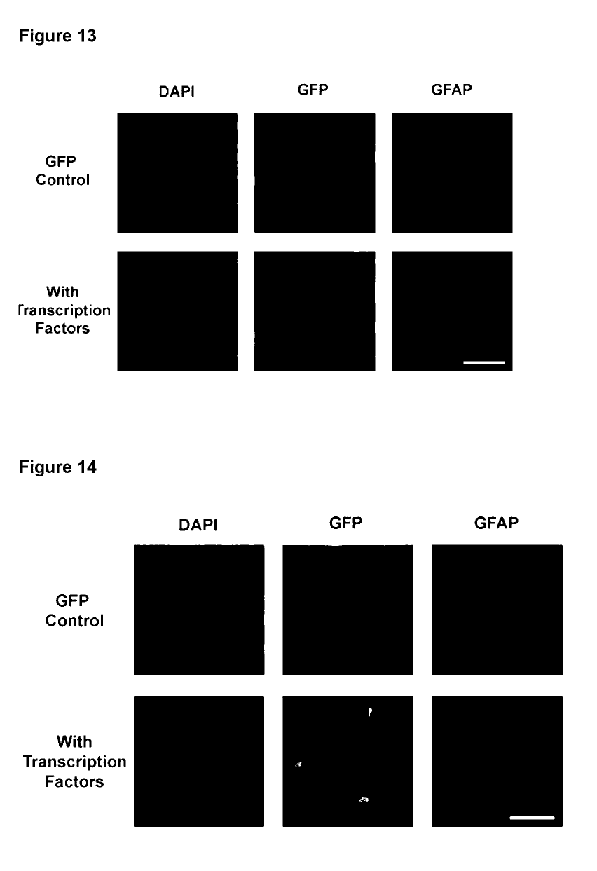

Immunofluorescence analysis shows expression of the astrocyte marker GFAP at day 21 of transdifferentiation (FIG. 13).

### Example 10—Embyronic Stem Cell (H9) to Astrocyte

Transcription Factors used: IRF1, SOX9, ARNT2, PAX6, SNAI2, RUNX2. (Mogrify also predicted the factor SOX5 but this was not used).

Transdifferentiation strategy: Human Embryonic Stem Cells (H9) was seeded onto matrigel-coated (BD Falcon) well plates at 5k cells/cm² 24 hours prior to viral transduction of transcription factors in Essential 8 medium (Life Technologies). On the following day, lentiviral particles encoding the transcription factors were transduced to cells in Essential 8 medium with Polybrene (Merck Millipore). Well plates were then centrifuged at 1900 rpm for 60 minutes immediately after transduction. At day 2, medium was replaced with N2 medium with B27 supplement (Life Technologies) and 0.6 μM CHIR99021 (Miltenyi Biotec). At day 6, medium was replaced with astrocyte medium (Life Technologies) supplemented with IL1β (10 ng/ml, Sigma-Aldrich). At day 8, medium was replaced with astrocyte medium. Medium was changed every 2 days throughout the experiment.

Immunofluorescence analysis shows expression of the astrocyte marker GFAP at day 21 of transdifferentiation (FIG. 14).

### Example 11—Pluripotent Stem Cell to Astrocyte

Transcription Factors used: PAX6, SNAI2, RUNX2, HMGB2. (Mogrify also predicted the factors POU3F2. E2F5 and SOX5 but these were not used).

Transdifferentiation strategy: Human Induced Pluripotent Stem Cells (32F donor) was seeded onto matrigel-coated (BD Falcon) well plates at 5k cells/cm² 24 hours prior to viral transduction of transcription factors in Essential 8 medium (Life Technologies). On the following day, lentiviral particles encoding the transcription factors were transduced to cells in Essential 8 medium with Polybrene (Merck Millipore). Well plates were then centrifuged at 1900 rpm for 60 minutes immediately after transduction. At day 2, medium was replaced with N2 medium with B27 supplement (Life Technologies) and 0.6 μM CHIR99021 (Miltenyi Biotec). At day 6, medium was replaced with astrocyte medium (Life Technologies) supplemented with IL1β (10 ng/ml, Sigma-Aldrich). At day 8, medium was replaced with astrocyte medium. Medium was changed every 2 days throughout the experiment.

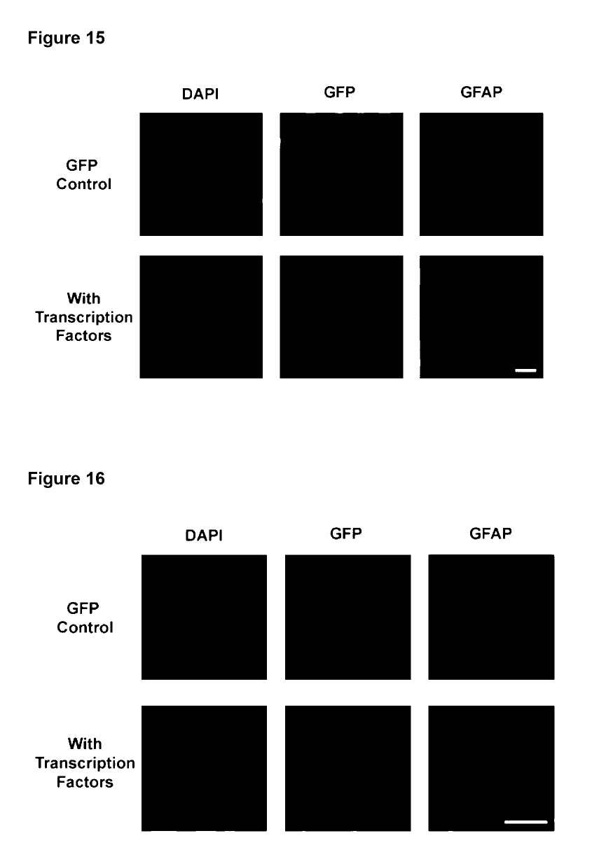

Immunofluorescence analysis showed expression of the astrocyte marker GFAP at day 21 of transdifferentiation (FIG. 15).

### Example 12—Mesenchymal Stem Cell to Astrocyte

Transcription Factors: SOX2, SOX9, ARNT2, MYBL2, E2F1, HMGB2. (Mogrify also identified the factor HOXB7 and JUNB but these were not used).

Transdifferentiation strategy: Bone Marrow Mesenchymal Stem Cells (7081 donor) was seeded onto well plates at 5k cells/cm² 24 hours prior to viral transduction of transcription factors in MSC medium(alpha-MEM with 15% FBS, glutamine, penicillin and streptomycin; Life Technologies). On the following day, lentiviral particles encoding the transcription factors were transduced to cells in MSC medium with Polybrene (Merck Millipore). Well plates were then centrifuged at 1900 rpm for 60 minutes immediately after transduction. At day 5, medium was replaced with astrocyte medium (Life Technologies) supplemented with IL1β (10 ng/ml, Sigma-Aldrich). At day 7, medium was replaced with astrocyte medium. Medium was changed every 2 days throughout the experiment.

Immunofluorescence analysis showed expression of the astrocyte marker GFAP at day 21 of transdifferentiation (FIG. 16).

### Example 13—Embryonic Stem Cell to Keratinocytes

Transcription Factors used: SOX9, NFKB1, MYC, FOSL2. (Mogrify also predicted the factors NR2F2, FOSL1 and AHR but these were not used).

Transdifferentiation strategy: Human Embryonic Stem Cells (H9) was seeded onto matrigel-coated (BD Falcon) well plates at 5k cells/cm² 24 hours prior to viral transduction of transcription factors in Essential 8 medium (Life Technologies). On the following day, lentiviral particles encoding the transcription factors were transduced to cells in Essential 8 medium with Polybrene (Merck Millipore). Well plates were then centrifuged at 1900 rpm for 60 minutes immediately after transduction. At day 2, medium was replaced with Essential 8 medium with 3 μM of retinoic acid. At day 6, medium was replaced with EpiLife medium (Life Technologies) supplemented BMP4 (50 ng/ml, Miltenyi Biotec) and EGF (5 ng/ml, Miltenyi Biotec). Medium was changed every 2 days throughout the experiment.

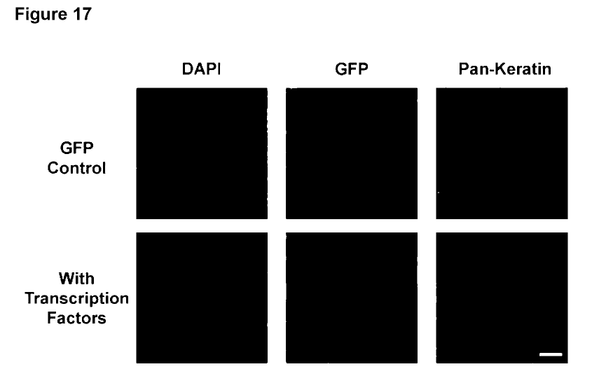

Immunofluorescence analysis showed expression of the keratinocyte marker Pan-Keratin at day 21 of transdifferentiation (FIG. 17).

### Example 14—Pluripotent Stem Cell to Keratinocytes

Transcription Factors: TFAP2A, MYC, SOX9, NFKB1. (Mogrify also predicted the factors TP63 and NFKBIA but these were not used).

Transdifferentiation strategy: Human Induced Pluripotent Stem Cells (32F donor) was seeded onto matrigel-coated (BD Falcon) well plates at 5k cells/cm² 24 hours prior to viral transduction of transcription factors in Essential 8 medium (Life Technologies). On the following day, lentiviral particles encoding the transcription factors were transduced to cells in Essential 8 medium with Polybrene (Merck Millipore). Well plates were then centrifuged at 1900 rpm for 60 minutes immediately after transduction. At day 2, medium was replaced with Essential 8 medium with 3 μM of retinoic acid. At day 6, medium was replaced with EpiLife medium (Life Technologies) supplemented BMP4 (50 ng/ml, Miltenyi Biotec) and EGF (5 ng/ml, Miltenyi Biotec). Medium was changed every 2 days throughout the experiment.

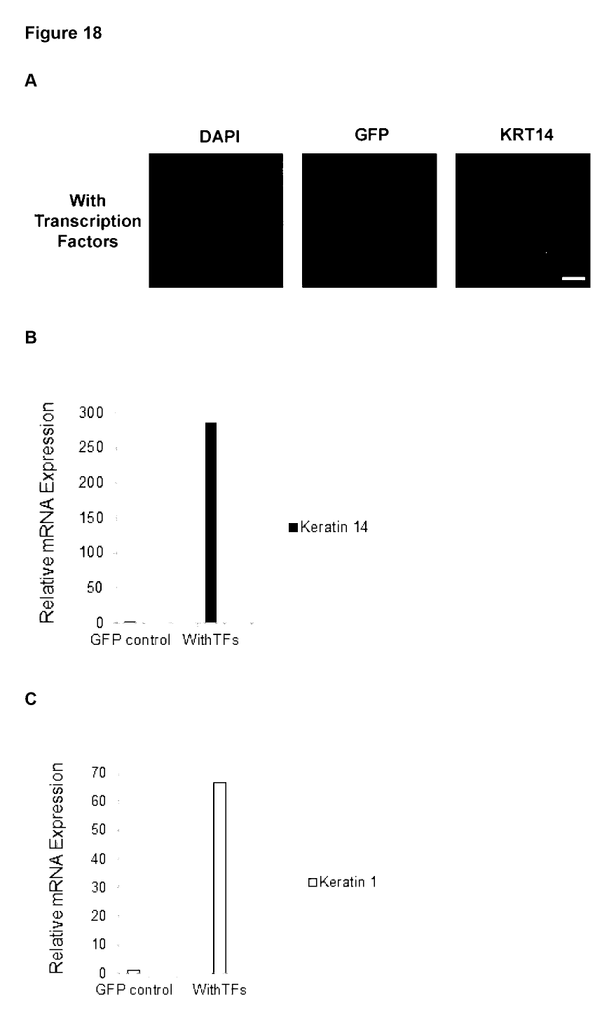

Immunofluorescence analysis shows expression of the keratinocyte marker Keratin 14 (KRT14) at day 21 of transdifferentiation (FIG. 18A).

qPCR analysis shows expression levels of the keratinocyte associated genes Keratin 14 and Keratin 1 at day 21 of transdifferentiation (FIGS. 18B and 18C).

### Example 15

Several attempts have been made to produce a representative cellular landscape but have focused on one or two cell types and are based on path-integral quasi-potentials, mechanistic modeling or probability landscapes. The inventors hypothesised that comparing all-against-all TF network differences as determined by Mogrify in combination with the transcriptional profiles would allow the creation of a 3D landscape representing human cell type (FIG. 7). The landscape places those cell types that are molecularly similar close together in the x-y plane, and adjusts the height (z direction) according to how likely a cell type is to be a good starting cell source (see online materials and methods for details). Interestingly, we observe that different stem cells are placed in the highest locations. This may suggest that the transcriptional networks of those cells at the highest points in the landscape are controlled by fewer TFs, and that the more differentiated the cell becomes (in the valleys) the more TFs are needed to fine tune the transcriptional network.

1. A method for determining the transcription factors required for conversion of a source cell to a cell exhibiting at least one characteristic of a target cell type, the method comprising the steps of:
determining differential expression of genes in the source and target cell types;
determining a network score for each transcription factor (TF) in each of the source and target cell types based on the differential gene expression over at least one network, wherein the network contains information of interactions that affect gene expression;
ranking the TFs based on a combination of network scores and differential gene expression information, thereby identifying the set of transcription factors for a conversion from a source cell to a cell exhibiting at least one characteristic of a target cell type.

2. A method according to claim 1, wherein a gene score is determined for each differentially expressed gene in the source and target cell types.

3. A method according to claim 1 or 2, wherein the gene score is a combination of the log fold change and adjusted P− value of the differential expression.

4. A method according to any one of claims 1 to 3, wherein the gene score is calculated using a tree-based method, preferably against a background.

5. A method according to any one of claims 1 to 4, wherein the network contains information of protein-DNA interactions, protein-DNA and/or protein-RNA interactions.

6. A method according to claim 5, wherein the network contains information of the interaction between transcription factors and regulatory regions of a gene.

7. A method according to claim 6, wherein the regulatory region is a promoter region of a gene.

8. A method according to any one of claims 1 to 7, wherein the method further comprises the step of collecting expression data for each gene prior to determining a gene score.

9. A method according to any one of claims 1 to 8, wherein the method further comprises the step of removing transcriptionally redundant TFs from the ranked lists from each cell type.

10. A method for determining the transcriptions factors required for conversion of a source cell to a cell exhibiting at least one characteristic of a target cell type, the method comprising the steps of:
collecting expression data for each gene in the source cell type and target cell type;
calculating the differential expression against a tree-based background for each gene in each sample then combine the log fold change and adjusted P− value to a gene score;
calculating a network score for each TF by performing a weighted sum of gene scores over at least one subnetwork centered on each TF;
ranking the TFs based on a combination of gene and network scores;
calculating the set of transcription factors for a conversion between any two cell types based on comparisons of ranked lists from each cell type; and optionally
removing transcriptionally redundant TFs from the lists.
thereby determining the transcription factors required for conversion of a source cell type to a target cell type.

11. A method for reprogramming a source cell, the method comprising increasing the protein expression of one or transcription factors, or variant thereof, in the source cell, wherein the source cell is reprogrammed to exhibit at least one characteristic of a target cell, wherein:
the source cell is selected from the group consisting of dermal fibroblasts, epidermal keratinocytes, embryonic stem cells, monocytes or cardiac fibroblasts;
the target cell is selected from the group consisting of chondrocytes, hair follicles, CD4+ T cells, CD8+ T cells, NK-cells, haemopoeitic stem cells (HSC), mesenchymal stem cells (MSC) of adipose, mesenchymal stem cells (MSC) of bone marrow, oligodendrocytes, oligodendrocyte precursors, skeletal muscle cells, smooth muscle cells and fetal cardiomyocytes; and
the transcription factors are one or more of those listed in Table 4.

12. A method of generating a cell exhibiting at least one characteristic of a target cell from a source cell, the method comprising:
increasing the amount of one or more transcription factors, or variant thereof, in a source cell; and
culturing the source cell for a sufficient time and under conditions to allow differentiation to a target cell; thereby generating the cell exhibiting at least one characteristic of a target cell from a source cell, wherein:
the source cell is selected from the group consisting of dermal fibroblasts, epidermal keratinocytes, embryonic stem cells, monocytes or cardiac fibroblasts;
the target cell is selected from the group consisting of chondrocytes, hair follicles, CD4+ T cells, CD8+ T cells, NK (natural killer)-cells, haemopoeitic stem cells (HSC), mesenchymal stem cells (MSC) of adipose, mesenchymal stem cells (MSC) of bone marrow, oligodendrocytes, oligodendrocyte precursors, skeletal muscle cells, smooth muscle cells and fetal cardiomyocytes; and
the transcription factors are one or more of those listed in Table 4.

13. A method according to claim 12, wherein the amount of one or more transcription factors, or variant thereof, is increased in a source cell by contacting the source cell with an agent which increases the expression of the transcription factor.

14. A method according to claim 13, wherein the agent is selected from the group consisting of: a nucleotide sequence, a protein, an aptamer and small molecule, ribosome, RNAi agent and peptide-nucleic acid (PNA) and analogues or variants thereof.

15. A method according to any one of claims 12 to 14, wherein the amount of one or more transcription factors is increased by introducing at least one nucleic acid sequence encoding a transcription factor protein listed in Table 4.

16. A method according to any one of claims 12 to 15, wherein the source cell is a dermal fibroblast, and wherein
(a) the target cell is a chondrocyte cell and the transcription factors are any one or more of BARX1, PITX1, SMAD6, FOXC1, SIX2 and AHR;
(b) the target cell is a hair follicle and the transcription factors are any one or more of ZIC1, PRRX2, RARB, VDR, FOXD1 and CREB3;
(c) the target cell is a CD4+ T cell and the transcription factors are any one or more of RORA, LEF1, JUN, FOS and BACH2;
(d) the target cell is a CD8+ T cell and the transcription factors are any one or more of RORA, FOS, SMAD7, JUN and RUNX3;
(e) the target cell is an NK cell and the transcription factors are any one or more of RORA, SMAD7, FOS, JUN and NFATC2;
(f) the target cell is a HSC and the transcription factors are any one or more of MYB, GATA1, GFI1 and GFI1B;
(g) the target cell is a MSC of adipose and the transcription factors are any one or more of NOTCH3, HIC1, ID1, ESRRA, IR1, SIX5, SREBF1 and SNAI2;
(h) the target cell is a MSC of bone marrow and the transcription factors are any one or more of SIX1, ID1, HOXA7, FOXC2, HOXA9, MAFB and IRX5;
(i) the target cell is a oligodendrocyte precursor and the transcription factors are any one or more of NKX2-1, ANKRD1, FOXA2, CDH1, ZFP42, IGF1, ICAM1 and FOS;
(j) the target cell is a skeletal muscle cell and the transcription factors are any one or more of MYOG, HIC1, MYOD1, FOXD1, PITX3, SIX2, HOXA7 and JUNB;
(k) the target cell is a smooth muscle cell and the transcription factors are any one or more of GATA6, LIF, JUNB, CREB3, MEIS1 and PBX1;
(l) the target cell is a fetal cardiomyocyte and the transcription factors are any one or more of BMP10, GATA6, TBX5, FHL2, NKX2-5, HAND2, GATA4 and PPARGC1A
(m) the target cell is an astrocyte and the transcription factors are any one or more of SOX2, SOX9, ARNT2, E2F5, PBX1, SMAD1 and RUNX2;
(n) the target cell is an epithelial cell and the transcription factors are any one or more of FOS, DBP, HES1, FOXA2, ESRRA, CDH1, FOXQ1 and PAX6;
(o) the target cell is an endothelial cell and the transcription factors are any one or more of SOX17, SMAD1, TAL1, IRF1, TCF7L1, MXD4 and JUNB; or
(p) the target cell is a keratinocyte and the transcription factors are any one or more of FOXQ1, SOX9, MAFB, CDH1, FOS and REL.

17. A method according to any one of claims 12 to 15, wherein the source cell is a epidermal keratinocyte, and wherein
(a) the target cell is a chondrocyte cell and the transcription factors are any one or more of BARX1, PITX1, SMAD6, TGFB3, FOXC1 and SIX2;
(b) the target cell is a hair follicle and the transcription factors are any one or more of RUNX1T1, ZIC1, PRRX1, MSX1, EBF1, FOXD1 and RUNX2;
(c) the target cell is a CD4+ T cell and the transcription factors are any one or more of RORA, LEF1, JUN, FOS and NR3C1;
(d) the target cell is a CD8+ T cell and the transcription factors are any one or more of RORA, FOS, SMAD7, JUN and RUNX3;
(e) the target cell is an NK cell and the transcription factors are any one or more of RORA, SMAD7, FOS, JUN, NFATC2 and RUNX3;
(f) the target cell is a HSC and the transcription factors are any one or more of MYB, GATA1, GFI1 and GFI1B;
(g) the target cell is a MSC of adipose and the transcription factors are any one or more of TWIST1, HIC1, ID1, MSX1, IRF1, HOXB7, SNAI2 and E2F1;
(h) the target cell is a MSC of bone marrow and the transcription factors are any one or more of SIX1, TWSIT1, ID1, HMOX1, FOXC2 and HOXA7;
(i) the target cell is a oligodendrocyte precursor cell and the transcription factors are any one or more of NKX2-1, ANKRD1, ZFP42, FOS, IGF1, ICAM1, FOXA2 and CDH1;
(j) the target cell is a skeletal muscle cell and the transcription factors are any one or more of MYOG, MYOD1, RF1, PITX3, HOXA7, FOXD1 and SOX8;
(k) the target cell is a smooth muscle cell and the transcription factors are any one or more of IRF1, GATA6, LIF and MEIS1;
(l) the target cell is an endothelial cell and the transcription factors are any one or more of SOX17, TAL1, SMAD1, IRF1, TCF7L1 and HOXB7; or
(m) the target cell is an epithelial cell and the transcription factors are any one or more of NOTCH1, HR, DBP, OTX1, ESRRA, FOXQ1, PAX6 and IRX5.

18. A method according to any one of claims 12 to 15, wherein the source cell is an embryonic stem cell, and wherein
(a) the target cell is a chondrocyte cell and the transcription factors are any one or more of BARX1, PITX1, SMAD6 and NFKB1;
(b) the target cell is a hair follicle and the transcription factors are any one or more of TWIST1, ZIC1, NR2F2, PRRX1, NFKB1 and AHR;
(c) the target cell is a CD4+ T cell and the transcription factors are any one or more of RORA, LEF1, JUN, FOS and BACH2;
(d) the target cell is a CD8+ T cell and the transcription factors are any one or more of RORA, FOS, SMAD7 and JUN;
(e) the target cell is an NK cell and the transcription factors are any one or more of RORA, SMAD7, FOS, JUN and NFATC2;
(f) the target cell is a HSC and the transcription factors are any one or more of MYB, IL1B, KLF1, GATA1, GFI1, GFI1B and NFE2;
(g) the target cell is a MSC of adipose and the transcription factors are any one or more of TWIST1, SNAI2, IRF1, MXD4, NFKB1, MSX1, HOXB7 and ESRRA;
(h) the target cell is a MSC of bone marrow and the transcription factors are any one or more of IRF1, RUNX1, CEBPB, AHR, FOXC2 and HOXA9;
(i) the target cell is a oligodendrocyte precursor cell and the transcription factors are any one or more of NKX2-1, ANKRD1, FOXA2, LMO3, FOS, IGF1, ICAM1 and CDH1;
(j) the target cell is a skeletal muscle cell and the transcription factors are any one or more of MYOG, IRF1, MYOD1, FOXD1, NFKB1, JUNB and HOXA7;
(k) the target cell is a smooth muscle cell and the transcription factors are any one or more of IRF1, NFKB1, JUNB, FOSL2, GATA6 and MEIS1;
(l) the target cell is an astrocyte and the transcription factors are any one or more of IRF1, SOX9, ARNT2, PAX6, SNAI2, RUNX2 and SOX5;
(m) the target cell is an endothelial cell and the transcription factors are any one or more of SOX17, SMAD1, TAL1, HOXB7, JUNB, NFKB1 and IRF1;
(n) the target cell is an epithelial cell and the transcription factors are any one or more of MYC, IL1B, FOS, NFKB1, ESRRA, FOXQ1, IRF1 and PAX6; or
(o) the target cell is a keratinocyte and the transcription factors are SOX9, NFKB1, MYC, NR2F2, FOSL2, FOSL1 and AHR.

19. A method according to any one of claims 12 to 15, wherein the source cell is a monocyte cell, and wherein
(a) the target cell is a HSC and the transcription factors are any one or more of MYB, IL1B, GATA1, GFI1 and GFI1B.

20. A method according to any one of claims 12 to 15, wherein the source cell is a cardiac fibroblast cell and the target cell is a fetal cardiomyocyte and the transcription factors are any one or more of BMP10, GATA6, TBX5, ANKRD1, HAND1, PPARGC1A, NKX2-5 and GATA4.

21. A method according to any one of claims 12 to 15, wherein the source cell is an mesenchymal stem cell, and the target cell is an astrocyte and the transcription factors are any one or more of SOX2, SOX9, ARNT2, MYBL2, POU3F2, E2F1 and HMGB2.

22. A method according to any one of claims 12 to 15, wherein the source cell is an pluripotent stem cell, and wherein
(a) the target cell is an astrocyte and the transcription factors are any one or more of PAX6, POU3F2, SNAI2, RUNX2, SOX5, E2F5 and HMGB2;
(b) the target cell is a keratinocyte and the transcription factors are any one or more of TP63, TFAP2A, MYC, NFKBIA, SOX9 and NFKB1; or
(c) the target cell is an endothelial cell and the transcription factors are any one or more of SOX17, TAL1, HOXB7, NFKB1, IRF1, SMAD1 and JUNB.

23. A method according to any one of claims 12 to 22, wherein the at least one characteristic of the target cell is up-regulation of any one or more target cell markers and/or change in cell morphology.

24. A method according to claim 23, wherein the markers for the following target cells include:
Chondrocytes: CD49. CD10, CD9, CD95, Integrin α10β1,105 and production of sulphated glycosaminoglycans (GAG);
Hair follicles: CD200, PHLDA1 and follistatin;
CD4+ T-cell: CD3, CD4;
CD8+ T-cell: CD3, CD8;
NK-cell: CD56, CD2;
HSCs: CD45, CD19/20, CD14/15, CD34, CD90;
MSCs of adipose: CD13, CD29, CD90, CD105, CD10, CD45 and differentiation in vitro towards osteoblasts, adipocytes and chondrocytes;
MSCs of bone marrow: CD13, CD29, CD90, CD105, CD10, and differentiation in vitro towards osteoblasts, adipocytes and chondrocytes;
Oligodendrocytes and oligodendrocyte precursor; NG2 and PDGFRα QPCR for Olig2 and Nkx2.2;
skeletal muscle cell: MyoD, Myogenin and Desmin;
smooth muscle cell: Myocardin, Smooth Muscle Alpha Actin and Smooth muscle myosin heavy chain;
fetal cardiomyocytes: MEF2C, MYH6, ACTN1, CDH2 and GJA1;
endothelial cell: PeCAM (CD31), VE-cadherin and VEGFR2;
keratinocytes: keratin1, keratin14, Pan-keratin and involucrin;
astrocyte: GFAP, 5100B and ALDH1L1; and
epithelial cells: cytokeratin 15 (CK15), cytokeratin 3 (CK3), involucrin and connexin 4.

25. A method according to any one of claims 12 to 24, wherein culturing the source cell for a sufficient time and under conditions to allow differentiation to a target cell includes culturing the cells for at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days the relevant medium shown in Table 9.

26. A method according to any one of claims 12 to 25, wherein the method further includes the step of administering the cell exhibiting at least one characteristic of a target cell type to an individual.

27. A cell exhibiting at least one characteristic of a target cell produced by a method according to any one of claims 12 to 26.

28. A population of cells, wherein at least 5% of cells exhibit at least one characteristic of the target cell and those cells are produced by a method according to any one of claims 12 to 26.

29. A population of cells according to claim 28, wherein at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% of the cells in the population exhibit at least one characteristic of the target cell.

30. A kit for use in a method according to any one of claims 12 to 26, for producing a cell exhibiting at least one characteristic of a target cell, the kit comprising one or more nucleic acids having one or more nucleic acid sequences a transcription factor described herein or variant thereof, optionally the kit further comprises instructions for reprogramming a source cell to a cell exhibiting at least one characteristic of a target cell.

31. A method for identifying an agent useful for promoting the conversion of a source cell type to a target cell type, the method comprising the steps of:
determining one or more transcription factors required for conversion of a source cell type to a target cell type by any method described herein;
screening one or more candidate agents for the ability to increase the amount of the one or more transcription factors required for conversion of a source cell type to a target cell type;
wherein an agent that increases the amount of the one or more transcription factors is an agent useful for promoting the conversion of a source cell type to a target cell type.


SEQUENCE LISTING (14 sequences)

SEQ ID NO: 1 - DNA, 20 bp, Artificial Sequence
  Primer sequence F-CD31
ccttctgctctgttcaagcc

SEQ ID NO: 2 - DNA, 20 bp, Artificial Sequence
  Primer sequence R-CD31
gggtcaggttcttcccattt

SEQ ID NO: 3 - DNA, 20 bp, Artificial Sequence
  Primer sequence F-VE
atgagaatgacaatgccccg

SEQ ID NO: 4 - DNA, 22 bp, Artificial Sequence
  Primer sequence R-VE
tgtctattgcggagatctgcag

SEQ ID NO: 5 - DNA, 21 bp, Artificial Sequence
  Primer sequence F-VEGFR2
ggcccaataatcagagtggca

SEQ ID NO: 6 - DNA, 23 bp, Artificial Sequence
  Primer sequence R-VEGFR2
ccagtgtcatttccgatcacttt

SEQ ID NO: 7 - DNA, 21 bp, Artificial Sequence
  Primer sequence F-KERATIN1
agagtggaccaactgaagagt

SEQ ID NO: 8 - DNA, 21 bp, Artificial Sequence
  Primer sequence R-KERATIN1
attctctgcatttgtccgctt

SEQ ID NO: 9 - DNA, 20 bp, Artificial Sequence
  Primer sequence F-KERATIN14
agaccaaaggtcgctactgc

SEQ ID NO: 10 - DNA, 20 bp, Artificial Sequence
  Primer sequence R-KERATIN14
aggagaactgggaggaggag

SEQ ID NO: 11 - DNA, 20 bp, Artificial Sequence
  Primer sequence F-INVOLUCRIN
ctgcctcagccttactgtga

SEQ ID NO: 12 - DNA, 21 bp, Artificial Sequence
  Primer sequence R-INVOLUCRIN
ggaggaggaacagtcttgagg

SEQ ID NO: 13 - DNA, 21 bp, Artificial Sequence
  Primer sequence F-beta-ACTIN
catgtacgttgctatccaggc

SEQ ID NO: 14 - DNA, 21 bp, Artificial Sequence
  Primer sequence R-beta- ACTIN
ctccttaatgtcacgcacgat